SEQUENCE LISTING (219 sequences)

SEQ ID NO: 1 - DNA, 54 bp, Artificial Sequence
  source: /note=“Description of Artificial Sequence Synthetic oligonucleotide”
aattgtggaacaagcaccaggaagtgaaaaagcaaaaagctttggaaaaacaga

SEQ ID NO: 2 - DNA, 63 bp, Artificial Sequence
  source: /note=“Description of Artificial Sequence Synthetic oligonucleotide”
aagggacaaaggatccatgtgggaccagttagaggatgcagctatggagaccttttctataag

SEQ ID NO: 3 - DNA, 19 bp, Artificial Sequence
  source: /note=“Description of Artificial Sequence Synthetic oligonucleotide”
cggcgaatctatgacaatc

SEQ ID NO: 4 - DNA, 70 bp, Artificial Sequence
  source: /note=“Description of Artificial Sequence Synthetic oligonucleotide”
ggttttcatagagctgaatcacattaaaaagtgcaatacagttcgaggcgtctttgtcctggaggaattt

SEQ ID NO: 5 - DNA, 19 bp, Artificial Sequence
  source: /note=“Description of Artificial Sequence Synthetic oligonucleotide”
agagctgaatcacattaaa

SEQ ID NO: 6 - DNA, 19 bp, Artificial Sequence
  source: /note=“Description of Artificial Sequence Synthetic oligonucleotide”
tttaaccgtttaccggcct

SEQ ID NO: 7 - DNA, 19 bp, Artificial Sequence
  source: /note=“Description of Artificial Sequence Synthetic oligonucleotide”
gtaccagcttcagtctttc

SEQ ID NO: 8 - DNA, 44 bp, Artificial Sequence
  source: /note=“Description of Artificial Sequence Synthetic primer”
cggaaccacagtacaagctttagcctctcaaacctcacactgcc

SEQ ID NO: 9 - DNA, 44 bp, Artificial Sequence
  source: /note=“Description of Artificial Sequence Synthetic primer”
ggcagtgtgaggtttgagaggctaaagcttgtactgtggttccg

SEQ ID NO: 10 - DNA, 23 bp, Artificial Sequence
  source: /note=“Description of Artificial Sequence Synthetic primer”
cgggactctgactccgaacctac

SEQ ID NO: 11 - DNA, 21 bp, Artificial Sequence
  source: /note=“Description of Artificial Sequence Synthetic primer”
gcggttcagcttgctcaggac

SEQ ID NO: 12 - DNA, 24 bp, Artificial Sequence
  source: /note=“Description of Artificial Sequence Synthetic primer”
ctgcaggtgaccctggaagggatc

SEQ ID NO: 13 - DNA, 27 bp, Artificial Sequence
  source: /note=“Description of Artificial Sequence Synthetic primer”
tgtacccgggtcagctagagacaggcc

SEQ ID NO: 14 - DNA, 4869 bp, Homo sapiens
actcagatcttccatggtgcaaaagaagaagttctgtcctcggttacttgactatctagtgatcgtaggggccaggcacccgagcagtgatagcgtggcccagactcctgaattgctacggcgataccccttggaggatcacactgagtttcccctgcccccagatgtagtgttcttctgccagcccgagggctgcctgagcgtgcggcagcggcgcatgagccttcgggatgatacctcttttgtcttcaccctcactgacaaggacactggagtcacgcgatatggcatctgtgttaacttctaccgctccttccaaaagcgaatctctaaggagaagggggaaggtggggcagggtcccgtgggaaggaaggaacccatgccacctgtgcctcagaagagggtggcactgagagctcagagagtggctcatccctgcagcctctcagtgctgactctacccctgatgtgaaccagtctcctcggggcaaacgccgggccaaggcggggagccgctcccgcaacagtactctcacgtccctgtgcgtgctcagccactaccctttcttctccaccttccgagagtgtttgtatactctcaagcgcctggtggactgctgtagtgagcgccttctgggcaagaaactgggcatccctcgaggcgtacaaagggacaccatgtggcggatctttactggatcgctgctggtagaggagaagtcaagtgcccttctgcatgaccttcgagagattgaggcctggatctatcgattgctgcgctccccagtacccgtctctgggcagaagcgagtagacatcgaggtcctaccccaagagctccagccagctctgacctttgctcttccagacccatctcgattcaccctagtggatttcccactgcaccttcccttggaacttctaggtgtggacgcctgtctccaggtgctaacctgcattctgttagagcacaaggtggtgctacagtcccgagactacaatgcactctccatgtctgtgatggcattcgtggcaatgatctacccactggaatatatgtttcctgtcatcccgctgctacccacctgcatggcatcagcagagcagctgctgttggctccaaccccgtacatcattggggttcctgccagcttcttcctctacaaactggacttcaaaatgcctgatgatgtatggctagtggatctggacagcaatagggtgattgcccccaccaatgcagaagtgctgcctatcctgccagaaccagaatcactagagctgaaaaagcatttaaagcaggccttggccagcatgagtctcaacacccagcccatcctcaatctggagaaatttcatgagggccaggagatcccccttctcttgggaaggccttctaatgacctgcagtccacaccgtccactgaattcaacccactcatctatggcaacgatgtggattctgtggatgttgcaaccagggttgccatggtacggttcttcaattccgccaacgtgctgcagggatttcagatgcacacgcgtaccctgcgcctctttcctcggcctgtggtagcttttcaagctggctcctttctagcctcacgtccccggcagactccttttgccgagaaattggccaggactcaggctgtggagtactttggggaatggatccttaaccccaccaactatgcctttcagcgaattcacaacaatatgtttgatccagccctgattggtgacaagccaaagtggtatgctcatcagctgcagcctatccactatcgcgtctatgacagcaattcccagctggctgaggccctgagtgtaccaccagagcgggactctgactccgaacctactgatgatagtggcagtgatagtatggattatgacgattcaagctcttcttactcctcccttggtgactttgtcagtgaaatgatgaaatgtgacattaatggtgatactcccaatgtggaccctctgacacatgcagcactgggggatgccagcgaggtggagattgacgagctgcagaatcagaaggaagcagaagagcctggcccagacagtgagaactctcaggaaaaccccccactgcgctccagctctagcaccacagccagcagcagccccagcactgtcatccacggagccaactctgaacctgctgactctacggagatggatgataaggcagcagtaggcgtctccaagcccctcccttccgtgcctcccagcattggcaaatcgaacgtggacagacgtcaggcagaaattggagaggggtcagtgcgccggcgaatctatgacaatccatacttcgagccccaatatggctttccccctgaggaagatgaggatgagcagggggaaagttacactccccgattcagccaacatgtcagtggcaatcgggctcaaaagctgctgcggcccaacagcttgagactggcaagtgactcagatgcagagtcagactctcgggcaagctctcccaactccaccgtctccaacaccagcaccgagggcttcgggggcatcatgtcttttgccagcagcctctatcggaaccacagtacaagctttagcctctcaaacctcacactgcccaccaaaggtgcccgagagaaggccacgcccttccccagtctgaaagtatttgggctaaatactctaatggagattgttactgaagccggccccgggagtggtgaaggaaacaggagggcgttagtggatcagaagtcatctgtcattaaacacagcccaacagtgaaaagagaacctccatcaccccagggtcgatccagcaattctagtgagaaccagcagttcctgaaggaggtggtgcacagcgtgctggacggccagggagttggctggctcaacatgaaaaaggtgcgccggctgctggagagcgagcagctgcgagtctttgtcctgagcaagctgaaccgcatggtgcagtcagaggacgatgcccggcaggacatcatcccggatgtggagatcagtcggaaggtgtacaagggaatgttagacctcctcaagtgtacagtcctcagcttggagcagtcctatgcccacgcgggtctgggtggcatggccagcatctttgggcttttggagattgcccagacccactactatagtaaagaaccagacaagcggaagagaagtccaacagaaagtgtaaataccccagttggcaaggatcctggcctagctgggcggggggacccaaaggctatggcacaactgagagttccacaactgggacctcgggcaccaagtgccacaggaaagggtcctaaggaactggacaccagaagtttaaaggaagaaaattttatagcatctattgaattgtggaacaagcaccaggaagtgaaaaagcaaaaagctttggaaaaacagaggcctgaagtaatcaaacctgtctttgaccttggtgagacagaggagaaaaagtcccagatcagcgcagacagtggtgtgagcctgacgtctagttcccagaggactgatcaagactctgtcatcggcgtgagtccagctgttatgatccgcagctcaagtcaggattctgaagttagcaccgtggtgagtaatagctctggagagacccttggagctgacagtgacttgagcagcaatgcaggtgatggaccaggtggcgagggcagtgttcacctggcaagctctcggggcactttgtctgatagtgaaattgagaccaactctgccacaagcaccatctttggtaaagcccacagcttgaagccaagcataaaggagaagctggcaggcagccccattcgtacttctgaagatgtgagccagcgagtctatctctatgagggactcctaggaagggacaaaggatccatgtgggaccagttagaggatgcagctatggagaccttttctataagcaaagagcgttctactttatgggaccaaatgcaattctgggaagatgccttcttagatgctgtgatgttggagagagaagggatgggtatggaccagggtccccaggaaatgatcgacaggtacctgtcccttggagaacatgaccggaagcgcctggaagatgatgaagatcgcttgctggccacacttctgcacaacctcatctcctacatgctgctgatgaaggtaaataagaatgacatccgcaagaaggtgaggcgcctaatgggaaagtcgcacattgggcttgtgtacagccagcaaatcaatgaggtgcttgatcagctggcgaacctgaatggacgcgatctctctatctggtccagtggcagccggcacatgaagaagcagacatttgtggtacatgcagggacagatacaaacggagatatctttttcatggaggtgtgcgatgactgtgtggtgttgcgtagtaacatcggaacagtgtatgagcgctggtggtacgagaagctcatcaacatgacctactgtcccaagacgaaggtgttgtgcttgtggcgtagaaatggctctgagacccagctcaacaagttctatactaaaaagtgtcgggagctgtactactgtgtgaaggacagcatggagcgcgctgccgcccgacagcaaagcatcaaacccggacctgaattgggtggcgagttccctgtgcaggacctgaagactggtgagggtggcctgctgcaggtgaccctggaagggatcaacctcaaattcatgcacaatcagttcctgaaattaaagaagtggtgagccacaagtacaagacaccaatggcccacgaaatctgctactccgtattatgtctcttctcgtacgtggctgcagttcatagcagtgaggaagatctcagaaccccgccc

SEQ ID NO: 15 - protein, 612 aa, Homo sapiens
LESEQLRVFVLSKLNRMVQSEDDARQDIIPDVEISRKVYKGMLDLLKCTVLSLEQSYAHAGLGGMASIFGLLEIAQTHYYSKEPDKRKRSPTESVNTPVGKDPGLAGRGDPKAMAQLRVPQLGPRAPSATGKGPKELDTRSLKEENFIASIELWNKHQEVKKQKALEKQRPEVIKPVFDLGETEEKKSQISADSGVSLTSSSQRTDQDSVIGVSPAVMIRSSSQDSEVSTVVSNSSGETLGADSDLSSNAGDGPGGEGSVHLASSRGTLSDSEIETNSATSTIFGKAHSLKPSIKEKLAGSPIRTSEDVSQRVYLYEGLLGRDKGSMWDQLEDAAMETFSISKERSTLWDQMQFWEDAFLDAVMLEREGMGMDQGPQEMIDRYLSLGEHDRKRLEDDEDRLLATLLHNLISYMLLMKVNKNDIRKKVRRLMGKSHIGLVYSQQINEVLDQLANLNGRDLSIWSSGSRHMKKQTFVVHAGTDTNGDIFFMEVCDDCVVLRSNIGTVYERWWYEKLINMTYCPKTKVLCLWRRNGSETQLNKFYTKKCRELYYCVKDSMERAAARQQSIKPGPELGGEFPVQDLKTGEGGLLQVTLEGINLKFMHNQFLKLKKW

SEQ ID NO: 16 - DNA, 5619 bp, Homo sapiens
cccgctgcccaggattggtagactccaccgctcggcagccggcttccctgctcggacgccgagcaccgccaaagcgcacttcgattttcagaattcctcctgggaatgctgactccttgcttggtgccctgatgcttctctgagataaactgatgaattggaaccatggtgcaaaagaagaagttctgtcctcggttacttgactatctagtgatcgtaggggccaggcacccgagcagtgatagcgtggcccagactcctgaattgctacggcgataccccttggaggatcacactgagtttcccctgcccccagatgtagtgttcttctgccagcccgagggctgcctgagcgtgcggcagcggcgcatgagccttcgggatgatacctcttttgtcttcaccctcactgacaaggacactggagtcacgcgatatggcatctgtgttaacttctaccgctccttccaaaagcgaatctctaaggggaagggggaaggtggggcagggtcccgtgggaaggaaggaacccatgccacctgtgcctcagaagagggtggcactgagagctcagagagtggctcatccctgcagcctttcagtgctgactctacccctgatgtgaaccagtctcctcggggcaaacgccgggccaaggcggggagccgctcccgcaacagtactctcacgtccctgtgcgtgctcagccactaccctttcttctccaccttccgagagtgtttgtatactctcaagcgcctggtggactgctgtagtgagcgccttctgggcaagaaactgggcatccctcgaggcgtacaaagggacaccatgtggcggatctttactggatcgctgctggtagaggagaagtcaagtgcccttctgcatgaccttcgagagattgaggcctggatctatcgattgctgcgctccccagtacccgtctctgggcagaagcgagtagacatcgaggtcctaccccaagagctccagccagctctgacctttgctcttccagacccatctcgattcaccctagtggatttcccactgcaccttcccttggaacttctaggtgtggacgcctgtctccagttgctaacctgcattctgttagagcacaaggtggtgctacagtcccgagactacaatgcactctccatgtctgtgatggcattcgtggcaatgatctacccactggagtatatgtttcctgtcatcccgctgctacccacctgcatggcatcagcagagcagctgctgttggctccaaccccgtacatcattggggttcctgccagcttcttcctctacaaactggacttcaaaatgcctgatgatgtatggctagtggatctggacagcaatagggtgattgcccccaccaatgcagaagtgctgcctatcctgccagaaccagaatcactagagctgaaaaagcatttaaagcaggccttggccagcatgagtctcaacacccagcccatcctcaatctggagaaatttcatgagggccaggagatcccccttctcttgggaaggccttctaatgacctgcagtccacaccgtccactgaattcaacccactcatctatggcaatgatgcggattctgtggatgttgcaaccagggttgccatggtacggttcttcaattccgccaacgtgctgcagggatttcagatgcacacgcgtaccctgcgcctctttcctcggcctgtggtagcttttcaagctggctcctttctagcctcacgtccccggcagactccttttgccgagaaattggccaggactcaggctgtggagtactttggggaatggatccttaaccccaccaactatgcctttcagcgaattcacaacaatatgtttgatccagccctgattggtgacaagccaaagtggtatgctcatcagctgcagcctatccactatcgcgtctatgacagcaattcccagctggctgaggccctgagtgtaccaccagagcgggactctgactccgaacctactgatgatagtggcagtgatagtatggattatgacgattcaagctcttcttactcctcccttggtgactttgtcagtgaaatgatgaaatgtgacattaatggtgatactcccaatgtggaccctctgacacatgcagcactgggggatgccagcgaggtggagattgacgagctgcagaatcagaaggaagcagaagagcctggcccagacagtgagaactctcaggaaaaccccccactgcgctccagctctagcaccacagccagcagcagccccagcactgtcatccacggagccaactctgaacctgctgactctacggagatggatgataaggcagcagtaggcgtctccaagcccctcccttccgtgcctcccagcattggcaaatcgaacgtggacagacgtcaggcagaaattggagagggggctcaaaagctgctgcggcccaacagcttgagactggcaagtgactcagatgcagagtcagactctcgggcaagctctcccaactccaccgtctccaacaccagcaccgagggcttcgggggcatcatgtcttttgccagcagcctctatcggaaccacagtaccagcttcagtctttcaaacctcacactgcccaccaaaggtgcccgagagaaggccacgcccttccccagtctgaaaggaaacaggagggcgttagtggatcagaagtcatctgtcattaaacacagcccaacagtgaaaagagaacctccatcaccccagggtcgatccagcaattctagtgagaaccagcagttcctgaaggaggtggtgcacagcgtgctggacggccagggagttggctggctcaacatgaaaaaggtgcgccggctgctggagagcgagcagctgcgagtctttgtcctgagcaagctgaaccgcatggtgcagtcagaggacgatgcccggcaggacatcatcccggatgtggagatcagtcggaaggtgtacaagggaatgttagacctcctcaagtgtacagtcctcagcttggagcagtcctatgcccacgcgggtctgggtggcatggccagcatctttgggcttttggagattgcccagacccactactatagtaaagaaccagacaagcggaagagaagtccaacagaaagtgtaaataccccagttggcaaggatcctggcctagctgggcggggggacccaaaggctatggcacaactgagagttccacaactgggacctcgggcaccaagtgccacaggaaagggtcctaaggaactggacaccagaagtttaaaggaagaaaattttatagcatctattgggcctgaagtaatcaaacctgtctttgaccttggtgagacagaggagaaaaagtcccagatcagcgcagacagtggtgtgagcctgacgtctagttcccagaggactgatcaagactctgtcatcggcgtgagtccagctgttatgatccgcagctcaagtcaggattctgaagttagcaccgtggtgagtaatagctctggagagacccttggagctgacagtgacttgagcagcaatgcaggtgatggaccaggtggcgagggcagtgttcacctggcaagctctcggggcactttgtctgatagtgaaattgagaccaactctgccacaagcaccatctttggtaaagcccacagcttgaagccatgcataaaggagaagctggcaggcagccccattcgtacttctgaagatgtgagccagcgagtctatctctatgagggactcctaggcaaagagcgttctactttatgggaccaaatgcaattctgggaagatgccttcttagatgctgtgatgttggagagagaagggatgggtatggaccagggtccccaggaaatgatcgacaggtacctgtcccttggagaacatgaccggaagcgcctggaagatgatgaagatcgcttgctggccacacttctgcacaacctcatctcctacatgctgctgatgaaggtaaataagaatgacatccgcaagaaggtgaggcgcctaatgggaaagtcgcacattgggcttgtgtacagccagcaaatcaatgaggtgcttgatcagctggcgaacctgaatggacgcgatctctctatctggtccagtggcagccggcacatgaagaagcagacatttgtggtacatgcagggacagatacaaacggagatatctttttcatggaggtgtgcgatgactgtgtggtgttgcgtagtaacatcggaacagtgtatgagcgctggtggtacgagaagctcatcaacatgacctactgtcccaagacgaaggtgttgtgcttgtggcgtagaaatggctctgagacccagctcaacaagttctatactaaaaagtgtcgggagctgtactactgtgtgaaggacagcatggagcgcgctgccgcccgacagcaaagcatcaaacccggacctgaattgggtggcgagttccctgtgcaggacctgaagactggtgagggtggcctgctgcaggtgaccctggaagggatcaacctcaaattcatgcacaatcagttcctgaaattaaagaagtggtgagccacaagtacaagacaccaatggcccacgaaatctgctactccgtattatgtctcttctcgtacgtggctgcagttcatagcagtgaggaagatctcagaaccccgccccggcctgtctctagctgatggagaggggctacgcagctgccccagcccagggcacgcccctggccccttgctgttcccaagtgcacgatgctgctgtgactgaggagtggatgatgctcgtgtgtcctctgcaacccccctgctgtggcttggttggttaccggttatgtgtccctctgagtgtgtcttgagcgtgtccaccttctccctctccactcccagaagaccaaactgccttcccctcagggctcaagaatgtgtacagtctgtggggccggtgtgaacccactattttgtgtccttgagacatttgtgttgtggttccttgtccttgtccctggcgttataactgtccactgcaagagtctggctctcccttctctgtgacccggcatgactgggcgcctggagcagttcactctgtgaggagtgagggaaccctggggctcaccctctcagaggaagggcacagagaggaagggaagaattggggggcagccggagtgagtggcagcctccctgcttccttctgcattcccaagccggcagccactgcccagggcccgcagtgttggctgctgcctgccacagcctctgtgactgcagtggagcggcgaattccctgtggcctgccacgccttcggcatcagaggatggagtggtcgaggctagtggagtcccagggaccgctggctgctctgcctgagcatcagggagggggcaggaaagaccaagctgggtttgcacatctgtctgcaggctgtctctccaggcacggggtgtcaggagggagagacagcctgggtatgggcaagaaatgactgtaaatatttcagccccacattatttatagaaaatgtacagttgtgtgaatgtgaaataaatgtcctcaattcccaaaaaa

SEQ ID NO: 17 - protein, 1479 aa, Homo sapiens [sequence omitted]

SEQ ID NO: 18 - DNA, 60 bp, Artificial Sequence
  source: /note=“Description of Artificial Sequence Synthetic oligonucleotide”
gatccccagagctgaatcacattaaattcaagagatttaatgtgattcagctctttttta

SEQ ID NO: 19 - DNA, 60 bp, Artificial Sequence
  source: /note=“Description of Artificial Sequence Synthetic oligonucleotide”
agcttaaaaaagagctgaatcacattaaatctcttgaatttaatgtgattcagctctggg

SEQ ID NO: 20 - DNA, 60 bp, Artificial Sequence
  source: /note=“Description of Artificial Sequence Synthetic oligonucleotide”
gatcccccagttcgaggcgtctttgtttcaagagaacaaagacgcctcgaactgttttta

SEQ ID NO: 21 - DNA, 60 bp, Artificial Sequence
  source: /note=“Description of Artificial Sequence Synthetic oligonucleotide”
agcttaaaaacagttcgaggcgtctttgttctcttgaaacaaagacgcctcgaactgggg

SEQ ID NO: 22 - DNA, 60 bp, Artificial Sequence
  source: /note=“Description of Artificial Sequence Synthetic oligonucleotide”
gatccccaggcgtctttgtcctggagttcaagagactccaggacaaagacgcctttttta

SEQ ID NO: 23 - DNA, 60 bp, Artificial Sequence
  source: /note=“Description of Artificial Sequence Synthetic oligonucleotide”
agcttaaaaaaggcgtctttgtcctggagtctcttgaactccaggacaaagacgcctggg

SEQ ID NO: 24 - RNA, 19 nt, Artificial Sequence
  source: /note=“Description of Artificial Sequence Synthetic oligonucleotide”
aauuguggaacaagcacca

SEQ ID NO: 25 - RNA, 19 nt, Artificial Sequence
  source: /note=“Description of Artificial Sequence Synthetic oligonucleotide”
uggugcuuguuccacaauu

SEQ ID NO: 26 - RNA, 19 nt, Artificial Sequence
  source: /note=“Description of Artificial Sequence Synthetic oligonucleotide”
auuguggaacaagcaccag

SEQ ID NO: 27 - RNA, 19 nt, Artificial Sequence
  source: /note=“Description of Artificial Sequence Synthetic oligonucleotide”
cuggugcuuguuccacaau

SEQ ID NO: 28 - RNA, 19 nt, Artificial Sequence
  source: /note=“Description of Artificial Sequence Synthetic oligonucleotide”
uuguggaacaagcaccagg

SEQ ID NO: 29 - RNA, 19 nt, Artificial Sequence
  source: /note=“Description of Artificial Sequence Synthetic oligonucleotide”
ccuggugcuuguuccacaa

SEQ ID NO: 30 - RNA, 19 nt, Artificial Sequence
  source: /note=“Description of Artificial Sequence Synthetic oligonucleotide”
uguggaacaagcaccagga

SEQ ID NO: 31 - RNA, 19 nt, Artificial Sequence
  source: /note=“Description of Artificial Sequence Synthetic oligonucleotide”
uccuggugcuuguuccaca

SEQ ID NO: 32 - RNA, 19 nt, Artificial Sequence
  source: /note=“Description of Artificial Sequence Synthetic oligonucleotide”
guggaacaagcaccaggaa

SEQ ID NO: 33 - RNA, 19 nt, Artificial Sequence
  source: /note=“Description of Artificial Sequence Synthetic oligonucleotide”
uuccuggugcuuguuccac

SEQ ID NO: 34 - RNA, 19 nt, Artificial Sequence
  source: /note=“Description of Artificial Sequence Synthetic oligonucleotide”
uggaacaagcaccaggaag

SEQ ID NO: 35 - RNA, 19 nt, Artificial Sequence
  source: /note=“Description of Artificial Sequence Synthetic oligonucleotide”
cuuccuggugcuuguucca

SEQ ID NO: 36 - RNA, 19 nt, Artificial Sequence
  source: /note=“Description of Artificial Sequence Synthetic oligonucleotide”
ggaacaagcaccaggaagu

SEQ ID NO: 37 - RNA, 19 nt, Artificial Sequence
  source: /note=“Description of Artificial Sequence Synthetic oligonucleotide”
acuuccuggugcuuguucc

SEQ ID NO: 38 - RNA, 19 nt, Artificial Sequence
  source: /note=“Description of Artificial Sequence Synthetic oligonucleotide”
gaacaagcaccaggaagug

SEQ ID NO: 39 - RNA, 19 nt, Artificial Sequence
  source: /note=“Description of Artificial Sequence Synthetic oligonucleotide”
cacuuccuggugcuuguuc

SEQ ID NO: 40 - RNA, 19 nt, Artificial Sequence
  source: /note=“Description of Artificial Sequence Synthetic oligonucleotide”
aacaagcaccaggaaguga

SEQ ID NO: 41 - RNA, 19 nt, Artificial Sequence
  source: /note=“Description of Artificial Sequence Synthetic oligonucleotide”
ucacuuccuggugcuuguu

SEQ ID NO: 42 - RNA, 19 nt, Artificial Sequence
  source: /note=“Description of Artificial Sequence Synthetic oligonucleotide”
acaagcaccaggaagugaa

SEQ ID NO: 43 - RNA, 19 nt, Artificial Sequence
  source: /note=“Description of Artificial Sequence Synthetic oligonucleotide”
uucacuuccuggugcuugu

SEQ ID NO: 44 - RNA, 19 nt, Artificial Sequence
  source: /note=“Description of Artificial Sequence Synthetic oligonucleotide”
caagcaccaggaagugaaa

SEQ ID NO: 45 - RNA, 19 nt, Artificial Sequence
  source: /note=“Description of Artificial Sequence Synthetic oligonucleotide”
uuucacuuccuggugcuug

SEQ ID NO: 46 - RNA, 19 nt, Artificial Sequence
  source: /note=“Description of Artificial Sequence Synthetic oligonucleotide”
aaacagaggccugaaguaa

SEQ ID NO: 47 - RNA, 19 nt, Artificial Sequence
  source: /note=“Description of Artificial Sequence Synthetic oligonucleotide”
uuacuucaggccucuguuu

SEQ ID NO: 48 - RNA, 19 nt, Artificial Sequence
  source: /note=“Description of Artificial Sequence Synthetic oligonucleotide”
aacagaggccugaaguaau

SEQ ID NO: 49 - RNA, 19 nt, Artificial Sequence
  source: /note=“Description of Artificial Sequence Synthetic oligonucleotide”
auuacuucaggccucuguu

SEQ ID NO: 50 - RNA, 19 nt, Artificial Sequence
  source: /note=“Description of Artificial Sequence Synthetic oligonucleotide”
acagaggccugaaguaauc

SEQ ID NO: 51 - RNA, 19 nt, Artificial Sequence
  source: /note=“Description of Artificial Sequence Synthetic oligonucleotide”
gauuacuucaggccucugu

SEQ ID NO: 52 - RNA, 19 nt, Artificial Sequence
  source: /note=“Description of Artificial Sequence Synthetic oligonucleotide”
cagaggccugaaguaauca

SEQ ID NO: 53 - RNA, 19 nt, Artificial Sequence
  source: /note=“Description of Artificial Sequence Synthetic oligonucleotide”
ugauuacuucaggccucug

SEQ ID NO: 54 - RNA, 19 nt, Artificial Sequence
  source: /note=“Description of Artificial Sequence Synthetic oligonucleotide”
agaggccugaaguaaucaa

SEQ ID NO: 55 - RNA, 19 nt, Artificial Sequence
  source: /note=“Description of Artificial Sequence Synthetic oligonucleotide”
uugauuacuucaggccucu

SEQ ID NO: 56 - RNA, 19 nt, Artificial Sequence
  source: /note=“Description of Artificial Sequence Synthetic oligonucleotide”
gaggccugaaguaaucaaa

SEQ ID NO: 57 - RNA, 19 nt, Artificial Sequence
  source: /note=“Description of Artificial Sequence Synthetic oligonucleotide”
uuugauuacuucaggccuc

SEQ ID NO: 58 - RNA, 19 nt, Artificial Sequence
  source: /note=“Description of Artificial Sequence Synthetic oligonucleotide”
gaagggacaaaggauccau

SEQ ID NO: 59 - RNA, 19 nt, Artificial Sequence
  source: /note=“Description of Artificial Sequence Synthetic oligonucleotide”
auggauccuuugucccuuc

SEQ ID NO: 60 - RNA, 19 nt, Artificial Sequence
  source: /note=“Description of Artificial Sequence Synthetic oligonucleotide”
aagggacaaaggauccaug

SEQ ID NO: 61 - RNA, 19 nt, Artificial Sequence
  source: /note=“Description of Artificial Sequence Synthetic oligonucleotide”
cauggauccuuugucccuu

SEQ ID NO: 62 - RNA, 19 nt, Artificial Sequence
  source: /note=“Description of Artificial Sequence Synthetic oligonucleotide”
agggacaaaggauccaugu

SEQ ID NO: 63 - RNA, 19 nt, Artificial Sequence
  source: /note=“Description of Artificial Sequence Synthetic oligonucleotide”
acauggauccuuugucccu

SEQ ID NO: 64 - RNA, 19 nt, Artificial Sequence
  source: /note=“Description of Artificial Sequence Synthetic oligonucleotide”
gggacaaaggauccaugug

SEQ ID NO: 65 - RNA, 19 nt, Artificial Sequence
  source: /note=“Description of Artificial Sequence Synthetic oligonucleotide”
cacauggauccuuuguccc

SEQ ID NO: 66 - RNA, 19 nt, Artificial Sequence
  source: /note=“Description of Artificial Sequence Synthetic oligonucleotide”
ggacaaaggauccaugugg

SEQ ID NO: 67 - RNA, 19 nt, Artificial Sequence
  source: /note=“Description of Artificial Sequence Synthetic oligonucleotide”
ccacauggauccuuugucc

SEQ ID NO: 68 - RNA, 19 nt, Artificial Sequence
  source: /note=“Description of Artificial Sequence Synthetic oligonucleotide”
gacaaaggauccauguggg

SEQ ID NO: 69 - RNA, 19 nt, Artificial Sequence
  source: /note=“Description of Artificial Sequence Synthetic oligonucleotide”
cccacauggauccuuuguc

SEQ ID NO: 70 - RNA, 19 nt, Artificial Sequence
  source: /note=“Description of Artificial Sequence Synthetic oligonucleotide”
acaaaggauccauguggga

SEQ ID NO: 71 - RNA, 19 nt, Artificial Sequence
  source: /note=“Description of Artificial Sequence Synthetic oligonucleotide”
ucccacauggauccuuugu

SEQ ID NO: 72 - RNA, 19 nt, Artificial Sequence
  source: /note=“Description of Artificial Sequence Synthetic oligonucleotide”
caaaggauccaugugggac

SEQ ID NO: 73 - RNA, 19 nt, Artificial Sequence
  source: /note=“Description of Artificial Sequence Synthetic oligonucleotide”
gucccacauggauccuuug

SEQ ID NO: 74 - RNA, 19 nt, Artificial Sequence
  source: /note=“Description of Artificial Sequence Synthetic oligonucleotide”
aaaggauccaugugggacc

SEQ ID NO: 75 - RNA, 19 nt, Artificial Sequence
  source: /note=“Description of Artificial Sequence Synthetic oligonucleotide”
ggucccacauggauccuuu

SEQ ID NO: 76 - RNA, 19 nt, Artificial Sequence
  source: /note=“Description of Artificial Sequence Synthetic oligonucleotide”
aaggauccaugugggacca

SEQ ID NO: 77 - RNA, 19 nt, Artificial Sequence
  source: /note=“Description of Artificial Sequence Synthetic oligonucleotide”
uggucccacauggauccuu

SEQ ID NO: 78 - RNA, 19 nt, Artificial Sequence
  source: /note=“Description of Artificial Sequence Synthetic oligonucleotide”
aggauccaugugggaccag

SEQ ID NO: 79 - RNA, 19 nt, Artificial Sequence
  source: /note=“Description of Artificial Sequence Synthetic oligonucleotide”
cuggucccacauggauccu

SEQ ID NO: 80 - RNA, 19 nt, Artificial Sequence
  source: /note=“Description of Artificial Sequence Synthetic oligonucleotide”
ggauccaugugggaccagu

SEQ ID NO: 81 - RNA, 19 nt, Artificial Sequence
  source: /note=“Description of Artificial Sequence Synthetic oligonucleotide”
acuggucccacauggaucc

SEQ ID NO: 82 - RNA, 19 nt, Artificial Sequence
  source: /note=“Description of Artificial Sequence Synthetic oligonucleotide”
gauccaugugggaccaguu

SEQ ID NO: 83 - RNA, 19 nt, Artificial Sequence
  source: /note=“Description of Artificial Sequence Synthetic oligonucleotide”
aacuggucccacauggauc

SEQ ID NO: 84 - RNA, 19 nt, Artificial Sequence
  source: /note=“Description of Artificial Sequence Synthetic oligonucleotide”
auccaugugggaccaguua

SEQ ID NO: 85 - RNA, 19 nt, Artificial Sequence
  source: /note=“Description of Artificial Sequence Synthetic oligonucleotide”
uaacuggucccacauggau

SEQ ID NO: 86 - RNA, 19 nt, Artificial Sequence
  source: /note=“Description of Artificial Sequence Synthetic oligonucleotide”
uccaugugggaccaguuag

SEQ ID NO: 87 - RNA, 19 nt, Artificial Sequence
  source: /note=“Description of Artificial Sequence Synthetic oligonucleotide”
cuaacuggucccacaugga

SEQ ID NO: 88 - RNA, 19 nt, Artificial Sequence
  source: /note=“Description of Artificial Sequence Synthetic oligonucleotide”
ccaugugggaccaguuaga

SEQ ID NO: 89 - RNA, 19 nt, Artificial Sequence
  source: /note=“Description of Artificial Sequence Synthetic oligonucleotide”
ucuaacuggucccacaugg

SEQ ID NO: 90 - RNA, 19 nt, Artificial Sequence
  source: /note=“Description of Artificial Sequence Synthetic oligonucleotide”
caugugggaccaguuagag

SEQ ID NO: 91 - RNA, 19 nt, Artificial Sequence
  source: /note=“Description of Artificial Sequence Synthetic oligonucleotide”
cucuaacuggucccacaug

SEQ ID NO: 92 - RNA, 19 nt, Artificial Sequence
  source: /note=“Description of Artificial Sequence Synthetic oligonucleotide”
augugggaccaguuagagg

SEQ ID NO: 93 - RNA, 19 nt, Artificial Sequence
  source: /note=“Description of Artificial Sequence Synthetic oligonucleotide”
ccucuaacuggucccacau

SEQ ID NO: 94 - RNA, 19 nt, Artificial Sequence
  source: /note=“Description of Artificial Sequence Synthetic oligonucleotide”
ugugggaccaguuagagga

SEQ ID NO: 95 - RNA, 19 nt, Artificial Sequence
  source: /note=“Description of Artificial Sequence Synthetic oligonucleotide”
uccucuaacuggucccaca

SEQ ID NO: 96 - RNA, 19 nt, Artificial Sequence
  source: /note=“Description of Artificial Sequence Synthetic oligonucleotide”
gugggaccaguuagaggau

SEQ ID NO: 97 - RNA, 19 nt, Artificial Sequence
  source: /note=“Description of Artificial Sequence Synthetic oligonucleotide”
auccucuaacuggucccac

SEQ ID NO: 98 - RNA, 19 nt, Artificial Sequence
  source: /note=“Description of Artificial Sequence Synthetic oligonucleotide”
ugggaccaguuagaggaug

SEQ ID NO: 99 - RNA, 19 nt, Artificial Sequence
  source: /note=“Description of Artificial Sequence Synthetic oligonucleotide”
cauccucuaacugguccca

SEQ ID NO: 100 - RNA, 19 nt, Artificial Sequence
  source: /note=“Description of Artificial Sequence Synthetic oligonucleotide”
gggaccaguuagaggaugc

SEQ ID NO: 101 - RNA, 19 nt, Artificial Sequence
  source: /note=“Description of Artificial Sequence Synthetic oligonucleotide”
gcauccucuaacugguccc

SEQ ID NO: 102 - RNA, 19 nt, Artificial Sequence
  source: /note=“Description of Artificial Sequence Synthetic oligonucleotide”
ggaccaguuagaggaugca

SEQ ID NO: 103 - RNA, 19 nt, Artificial Sequence
  source: /note=“Description of Artificial Sequence Synthetic oligonucleotide”
ugcauccucuaacuggucc

SEQ ID NO: 104 - RNA, 19 nt, Artificial Sequence
  source: /note=“Description of Artificial Sequence Synthetic oligonucleotide”
gaccaguuagaggaugcag

SEQ ID NO: 105 - RNA, 19 nt, Artificial Sequence
  source: /note=“Description of Artificial Sequence Synthetic oligonucleotide”
cugcauccucuaacugguc

SEQ ID NO: 106 - RNA, 19 nt, Artificial Sequence
  source: /note=“Description of Artificial Sequence Synthetic oligonucleotide”
accaguuagaggaugcagc

SEQ ID NO: 107 - RNA, 19 nt, Artificial Sequence
  source: /note=“Description of Artificial Sequence Synthetic oligonucleotide”
gcugcauccucuaacuggu

SEQ ID NO: 108 - RNA, 19 nt, Artificial Sequence
  source: /note=“Description of Artificial Sequence Synthetic oligonucleotide”
ccaguuagaggaugcagcu

SEQ ID NO: 109 - RNA, 19 nt, Artificial Sequence
  source: /note=“Description of Artificial Sequence Synthetic oligonucleotide”
agcugcauccucuaacugg

SEQ ID NO: 110 - RNA, 19 nt, Artificial Sequence
  source: /note=“Description of Artificial Sequence Synthetic oligonucleotide”
caguuagaggaugcagcua

SEQ ID NO: 111 - RNA, 19 nt, Artificial Sequence
  source: /note=“Description of Artificial Sequence Synthetic oligonucleotide”
uagcugcauccucuaacug

SEQ ID NO: 112 - RNA, 19 nt, Artificial Sequence
  source: /note=“Description of Artificial Sequence Synthetic oligonucleotide”
aguuagaggaugcagcuau

SEQ ID NO: 113 - RNA, 19 nt, Artificial Sequence
  source: /note=“Description of Artificial Sequence Synthetic oligonucleotide”
auagcugcauccucuaacu

SEQ ID NO: 114 - RNA, 19 nt, Artificial Sequence
  source: /note=“Description of Artificial Sequence Synthetic oligonucleotide”
guuagaggaugcagcuaug

SEQ ID NO: 115 - RNA, 19 nt, Artificial Sequence
  source: /note=“Description of Artificial Sequence Synthetic oligonucleotide”
cauagcugcauccucuaac

SEQ ID NO: 116 - RNA, 19 nt, Artificial Sequence
  source: /note=“Description of Artificial Sequence Synthetic oligonucleotide”
uuagaggaugcagcuaugg

SEQ ID NO: 117 - RNA, 19 nt, Artificial Sequence
  source: /note=“Description of Artificial Sequence Synthetic oligonucleotide”
ccauagcugcauccucuaa

SEQ ID NO: 118 - RNA, 19 nt, Artificial Sequence
  source: /note=“Description of Artificial Sequence Synthetic oligonucleotide”
uagaggaugcagcuaugga

SEQ ID NO: 119 - RNA, 19 nt, Artificial Sequence
  source: /note=“Description of Artificial Sequence Synthetic oligonucleotide”
uccauagcugcauccucua

SEQ ID NO: 120 - RNA, 19 nt, Artificial Sequence
  source: /note=“Description of Artificial Sequence Synthetic oligonucleotide”
agaggaugcagcuauggag

SEQ ID NO: 121 - RNA, 19 nt, Artificial Sequence
  source: /note=“Description of Artificial Sequence Synthetic oligonucleotide”
cuccauagcugcauccucu

SEQ ID NO: 122 - RNA, 19 nt, Artificial Sequence
  source: /note=“Description of Artificial Sequence Synthetic oligonucleotide”
gaggaugcagcuauggaga

SEQ ID NO: 123 - RNA, 19 nt, Artificial Sequence
  source: /note=“Description of Artificial Sequence Synthetic oligonucleotide”
ucuccauagcugcauccuc

SEQ ID NO: 124 - RNA, 19 nt, Artificial Sequence
  source: /note=“Description of Artificial Sequence Synthetic oligonucleotide”
aggaugcagcuauggagac

SEQ ID NO: 125 - RNA, 19 nt, Artificial Sequence
  source: /note=“Description of Artificial Sequence Synthetic oligonucleotide”
gucuccauagcugcauccu

SEQ ID NO: 126 - RNA, 19 nt, Artificial Sequence
  source: /note=“Description of Artificial Sequence Synthetic oligonucleotide”
ggaugcagcuauggagacc

SEQ ID NO: 127 - RNA, 19 nt, Artificial Sequence
  source: /note=“Description of Artificial Sequence Synthetic oligonucleotide”
ggucuccauagcugcaucc

SEQ ID NO: 128 - RNA, 19 nt, Artificial Sequence
  source: /note=“Description of Artificial Sequence Synthetic oligonucleotide”
gaugcagcuauggagaccu

SEQ ID NO: 129 - RNA, 19 nt, Artificial Sequence
  source: /note=“Description of Artificial Sequence Synthetic oligonucleotide”
aggucuccauagcugcauc

SEQ ID NO: 130 - RNA, 19 nt, Artificial Sequence
  source: /note=“Description of Artificial Sequence Synthetic oligonucleotide”
augcagcuauggagaccuu

SEQ ID NO: 131 - RNA, 19 nt, Artificial Sequence
  source: /note=“Description of Artificial Sequence Synthetic oligonucleotide”
aaggucuccauagcugcau

SEQ ID NO: 132 - RNA, 19 nt, Artificial Sequence
  source: /note=“Description of Artificial Sequence Synthetic oligonucleotide”
ugcagcuauggagaccuuu

SEQ ID NO: 133 - RNA, 19 nt, Artificial Sequence
  source: /note=“Description of Artificial Sequence Synthetic oligonucleotide”
aaaggucuccauagcugca

SEQ ID NO: 134 - RNA, 19 nt, Artificial Sequence
  source: /note=“Description of Artificial Sequence Synthetic oligonucleotide”
uuucuauaagcaaagagcg

SEQ ID NO: 135 - RNA, 19 nt, Artificial Sequence
  source: /note=“Description of Artificial Sequence Synthetic oligonucleotide”
cgcucuuugcuuauagaaa

SEQ ID NO: 136 - RNA, 19 nt, Artificial Sequence
  source: /note=“Description of Artificial Sequence Synthetic oligonucleotide”
uucuauaagcaaagagcgu

SEQ ID NO: 137 - RNA, 19 nt, Artificial Sequence
  source: /note=“Description of Artificial Sequence Synthetic oligonucleotide”
acgcucuuugcuuauagaa

SEQ ID NO: 138 - RNA, 19 nt, Artificial Sequence
  source: /note=“Description of Artificial Sequence Synthetic oligonucleotide”
ucuauaagcaaagagcguu

SEQ ID NO: 139 - RNA, 19 nt, Artificial Sequence
  source: /note=“Description of Artificial Sequence Synthetic oligonucleotide”
aacgcucuuugcuuauaga

SEQ ID NO: 140 - RNA, 19 nt, Artificial Sequence
  source: /note=“Description of Artificial Sequence Synthetic oligonucleotide”
cuauaagcaaagagcguuc

SEQ ID NO: 141 - RNA, 19 nt, Artificial Sequence
  source: /note=“Description of Artificial Sequence Synthetic oligonucleotide”
gaacgcucuuugcuuauag

SEQ ID NO: 142 - RNA, 19 nt, Artificial Sequence
  source: /note=“Description of Artificial Sequence Synthetic oligonucleotide”
uauaagcaaagagcguucu

SEQ ID NO: 143 - RNA, 19 nt, Artificial Sequence
  source: /note=“Description of Artificial Sequence Synthetic oligonucleotide”
agaacgcucuuugcuuaua

SEQ ID NO: 144 - RNA, 19 nt, Artificial Sequence
  source: /note=“Description of Artificial Sequence Synthetic oligonucleotide”
auaagcaaagagcguucua

SEQ ID NO: 145 - RNA, 19 nt, Artificial Sequence
  source: /note=“Description of Artificial Sequence Synthetic oligonucleotide”
uagaacgcucuuugcuuau

SEQ ID NO: 146 - RNA, 19 nt, Artificial Sequence
  source: /note=“Description of Artificial Sequence Synthetic oligonucleotide”
uaagcaaagagcguucuac

SEQ ID NO: 147 - RNA, 19 nt, Artificial Sequence
  source: /note=“Description of Artificial Sequence Synthetic oligonucleotide”
guagaacgcucuuugcuua

SEQ ID NO: 148 - RNA, 19 nt, Artificial Sequence
  source: /note=“Description of Artificial Sequence Synthetic oligonucleotide”
aagcaaagagcguucuacu

SEQ ID NO: 149 - RNA, 19 nt, Artificial Sequence
  source: /note=“Description of Artificial Sequence Synthetic oligonucleotide”
aguagaacgcucuuugcuu

SEQ ID NO: 150 - RNA, 19 nt, Artificial Sequence
  source: /note=“Description of Artificial Sequence Synthetic oligonucleotide”
agcaaagagcguucuacuu

SEQ ID NO: 151 - RNA, 19 nt, Artificial Sequence
  source: /note=“Description of Artificial Sequence Synthetic oligonucleotide”
aaguagaacgcucuuugcu

SEQ ID NO: 152 - RNA, 19 nt, Artificial Sequence
  source: /note=“Description of Artificial Sequence Synthetic oligonucleotide”
aaagugcaauacaguucga

SEQ ID NO: 153 - RNA, 19 nt, Artificial Sequence
  source: /note=“Description of Artificial Sequence Synthetic oligonucleotide”
ucgaacuguauugcacuuu

SEQ ID NO: 154 - RNA, 19 nt, Artificial Sequence
  source: /note=“Description of Artificial Sequence Synthetic oligonucleotide”
aagugcaauacaguucgag

SEQ ID NO: 155 - RNA, 19 nt, Artificial Sequence
  source: /note=“Description of Artificial Sequence Synthetic oligonucleotide”
cucgaacuguauugcacuu

SEQ ID NO: 156 - RNA, 19 nt, Artificial Sequence
  source: /note=“Description of Artificial Sequence Synthetic oligonucleotide”
agugcaauacaguucgagg

SEQ ID NO: 157 - RNA, 19 nt, Artificial Sequence
  source: /note=“Description of Artificial Sequence Synthetic oligonucleotide”
ccucgaacuguauugcacu

SEQ ID NO: 158 - RNA, 19 nt, Artificial Sequence
  source: /note=“Description of Artificial Sequence Synthetic oligonucleotide”
gugcaauacaguucgaggc

SEQ ID NO: 159 - RNA, 19 nt, Artificial Sequence
  source: /note=“Description of Artificial Sequence Synthetic oligonucleotide”
gccucgaacuguauugcac

SEQ ID NO: 160 - RNA, 19 nt, Artificial Sequence
  source: /note=“Description of Artificial Sequence Synthetic oligonucleotide”
ugcaauacaguucgaggcg

SEQ ID NO: 161 - RNA, 19 nt, Artificial Sequence
  source: /note=“Description of Artificial Sequence Synthetic oligonucleotide”
cgccucgaacuguauugca

SEQ ID NO: 162 - RNA, 19 nt, Artificial Sequence
  source: /note=“Description of Artificial Sequence Synthetic oligonucleotide”
gcaauacaguucgaggcgu

SEQ ID NO: 163 - RNA, 19 nt, Artificial Sequence
  source: /note=“Description of Artificial Sequence Synthetic oligonucleotide”
acgccucgaacuguauugc

SEQ ID NO: 164 - RNA, 19 nt, Artificial Sequence
  source: /note=“Description of Artificial Sequence Synthetic oligonucleotide”
caauacaguucgaggcguc

SEQ ID NO: 165 - RNA, 19 nt, Artificial Sequence
  source: /note=“Description of Artificial Sequence Synthetic oligonucleotide”
gacgccucgaacuguauug

SEQ ID NO: 166 - RNA, 19 nt, Artificial Sequence
  source: /note=“Description of Artificial Sequence Synthetic oligonucleotide”
aauacaguucgaggcgucu

SEQ ID NO: 167 - RNA, 19 nt, Artificial Sequence
  source: /note=“Description of Artificial Sequence Synthetic oligonucleotide”
agacgccucgaacuguauu

SEQ ID NO: 168 - RNA, 19 nt, Artificial Sequence
  source: /note=“Description of Artificial Sequence Synthetic oligonucleotide”
auacaguucgaggcgucuu

SEQ ID NO: 169 - RNA, 19 nt, Artificial Sequence
  source: /note=“Description of Artificial Sequence Synthetic oligonucleotide”
aagacgccucgaacuguau

SEQ ID NO: 170 - RNA, 19 nt, Artificial Sequence
  source: /note=“Description of Artificial Sequence Synthetic oligonucleotide”
uacaguucgaggcgucuuu

SEQ ID NO: 171 - RNA, 19 nt, Artificial Sequence
  source: /note=“Description of Artificial Sequence Synthetic oligonucleotide”
aaagacgccucgaacugua

SEQ ID NO: 172 - RNA, 19 nt, Artificial Sequence
  source: /note=“Description of Artificial Sequence Synthetic oligonucleotide”
acaguucgaggcgucuuug

SEQ ID NO: 173 - RNA, 19 nt, Artificial Sequence
  source: /note=“Description of Artificial Sequence Synthetic oligonucleotide”
caaagacgccucgaacugu

SEQ ID NO: 174 - RNA, 19 nt, Artificial Sequence
  source: /note=“Description of Artificial Sequence Synthetic oligonucleotide”
caguucgaggcgucuuugu

SEQ ID NO: 175 - RNA, 19 nt, Artificial Sequence
  source: /note=“Description of Artificial Sequence Synthetic oligonucleotide”
acaaagacgccucgaacug

SEQ ID NO: 176 - RNA, 19 nt, Artificial Sequence
  source: /note=“Description of Artificial Sequence Synthetic oligonucleotide”
aguucgaggcgucuuuguc

SEQ ID NO: 177 - RNA, 19 nt, Artificial Sequence
  source: /note=“Description of Artificial Sequence Synthetic oligonucleotide”
gacaaagacgccucgaacu

SEQ ID NO: 178 - RNA, 19 nt, Artificial Sequence
  source: /note=“Description of Artificial Sequence Synthetic oligonucleotide”
guucgaggcgucuuugucc

SEQ ID NO: 179 - RNA, 19 nt, Artificial Sequence
  source: /note=“Description of Artificial Sequence Synthetic oligonucleotide”
ggacaaagacgccucgaac

SEQ ID NO: 180 - RNA, 19 nt, Artificial Sequence
  source: /note=“Description of Artificial Sequence Synthetic oligonucleotide”
uucgaggcgucuuuguccu

SEQ ID NO: 181 - RNA, 19 nt, Artificial Sequence
  source: /note=“Description of Artificial Sequence Synthetic oligonucleotide”
aggacaaagacgccucgaa

SEQ ID NO: 182 - RNA, 19 nt, Artificial Sequence
  source: /note=“Description of Artificial Sequence Synthetic oligonucleotide”
ucgaggcgucuuuguccug

SEQ ID NO: 183 - RNA, 19 nt, Artificial Sequence
  source: /note=“Description of Artificial Sequence Synthetic oligonucleotide”
caggacaaagacgccucga

SEQ ID NO: 184 - RNA, 19 nt, Artificial Sequence
  source: /note=“Description of Artificial Sequence Synthetic oligonucleotide”
cgaggcgucuuuguccugg

SEQ ID NO: 185 - RNA, 19 nt, Artificial Sequence
  source: /note=“Description of Artificial Sequence Synthetic oligonucleotide”
ccaggacaaagacgccucg

SEQ ID NO: 186 - RNA, 19 nt, Artificial Sequence
  source: /note=“Description of Artificial Sequence Synthetic oligonucleotide”
gaggcgucuuuguccugga

SEQ ID NO: 187 - RNA, 19 nt, Artificial Sequence
  source: /note=“Description of Artificial Sequence Synthetic oligonucleotide”
uccaggacaaagacgccuc

SEQ ID NO: 188 - RNA, 19 nt, Artificial Sequence
  source: /note=“Description of Artificial Sequence Synthetic oligonucleotide”
aggcgucuuuguccuggag

SEQ ID NO: 189 - RNA, 19 nt, Artificial Sequence
  source: /note=“Description of Artificial Sequence Synthetic oligonucleotide”
cuccaggacaaagacgccu

SEQ ID NO: 190 - RNA, 19 nt, Artificial Sequence
  source: /note=“Description of Artificial Sequence Synthetic oligonucleotide”
ggcgucuuuguccuggagg

SEQ ID NO: 191 - RNA, 19 nt, Artificial Sequence
  source: /note=“Description of Artificial Sequence Synthetic oligonucleotide”
ccuccaggacaaagacgcc

SEQ ID NO: 192 - RNA, 19 nt, Artificial Sequence
  source: /note=“Description of Artificial Sequence Synthetic oligonucleotide”
gcgucuuuguccuggagga

SEQ ID NO: 193 - RNA, 19 nt, Artificial Sequence
  source: /note=“Description of Artificial Sequence Synthetic oligonucleotide”
uccuccaggacaaagacgc

SEQ ID NO: 194 - RNA, 19 nt, Artificial Sequence
  source: /note=“Description of Artificial Sequence Synthetic oligonucleotide”
cgucuuuguccuggaggaa

SEQ ID NO: 195 - RNA, 19 nt, Artificial Sequence
  source: /note=“Description of Artificial Sequence Synthetic oligonucleotide”
uuccuccaggacaaagacg

SEQ ID NO: 196 - RNA, 19 nt, Artificial Sequence
  source: /note=“Description of Artificial Sequence Synthetic oligonucleotide”
gucuuuguccuggaggaau

SEQ ID NO: 197 - RNA, 19 nt, Artificial Sequence
  source: /note=“Description of Artificial Sequence Synthetic oligonucleotide”
auuccuccaggacaaagac

SEQ ID NO: 198 - RNA, 19 nt, Artificial Sequence
  source: /note=“Description of Artificial Sequence Synthetic oligonucleotide”
ucuuuguccuggaggaauu

SEQ ID NO: 199 - RNA, 19 nt, Artificial Sequence
  source: /note=“Description of Artificial Sequence Synthetic oligonucleotide”
aauuccuccaggacaaaga

SEQ ID NO: 200 - RNA, 19 nt, Artificial Sequence
  source: /note=“Description of Artificial Sequence Synthetic oligonucleotide”
cuuuguccuggaggaauuu

SEQ ID NO: 201 - RNA, 19 nt, Artificial Sequence
  source: /note=“Description of Artificial Sequence Synthetic oligonucleotide”
aaauuccuccaggacaaag

SEQ ID NO: 202 - RNA, 19 nt, Artificial Sequence
  source: /note=“Description of Artificial Sequence Synthetic oligonucleotide”
uuuguccuggaggaauuug

SEQ ID NO: 203 - RNA, 19 nt, Artificial Sequence
  source: /note=“Description of Artificial Sequence Synthetic oligonucleotide”
caaauuccuccaggacaaa

SEQ ID NO: 204 - RNA, 19 nt, Artificial Sequence
  source: /note=“Description of Artificial Sequence Synthetic oligonucleotide”
uuguccuggaggaauuugu

SEQ ID NO: 205 - RNA, 19 nt, Artificial Sequence
  source: /note=“Description of Artificial Sequence Synthetic oligonucleotide”
acaaauuccuccaggacaa

SEQ ID NO: 206 - RNA, 19 nt, Artificial Sequence
  source: /note=“Description of Artificial Sequence Synthetic oligonucleotide”
uguccuggaggaauuuguu

SEQ ID NO: 207 - RNA, 19 nt, Artificial Sequence
  source: /note=“Description of Artificial Sequence Synthetic oligonucleotide”
aacaaauuccuccaggaca

SEQ ID NO: 208 - RNA, 19 nt, Artificial Sequence
  source: /note=“Description of Artificial Sequence Synthetic oligonucleotide”
guccuggaggaauuuguuc

SEQ ID NO: 209 - RNA, 19 nt, Artificial Sequence
  source: /note=“Description of Artificial Sequence Synthetic oligonucleotide”
gaacaaauuccuccaggac

SEQ ID NO: 210 - RNA, 19 nt, Artificial Sequence
  source: /note=“Description of Artificial Sequence Synthetic oligonucleotide”
uccuggaggaauuuguucc

SEQ ID NO: 211 - RNA, 19 nt, Artificial Sequence
  source: /note=“Description of Artificial Sequence Synthetic oligonucleotide”
ggaacaaauuccuccagga

SEQ ID NO: 212 - RNA, 19 nt, Artificial Sequence
  source: /note=“Description of Artificial Sequence Synthetic oligonucleotide”
ccuggaggaauuuguuccu

SEQ ID NO: 213 - RNA, 19 nt, Artificial Sequence
  source: /note=“Description of Artificial Sequence Synthetic oligonucleotide”
aggaacaaauuccuccagg

SEQ ID NO: 214 - RNA, 19 nt, Artificial Sequence
  source: /note=“Description of Artificial Sequence Synthetic oligonucleotide”
cuggaggaauuuguuccug

SEQ ID NO: 215 - RNA, 19 nt, Artificial Sequence
  source: /note=“Description of Artificial Sequence Synthetic oligonucleotide”
caggaacaaauuccuccag

SEQ ID NO: 216 - RNA, 19 nt, Artificial Sequence
  source: /note=“Description of Artificial Sequence Synthetic oligonucleotide”
uggaggaauuuguuccuga

SEQ ID NO: 217 - RNA, 19 nt, Artificial Sequence
  source: /note=“Description of Artificial Sequence Synthetic oligonucleotide”
ucaggaacaaauuccucca

SEQ ID NO: 218 - RNA, 19 nt, Artificial Sequence
  source: /note=“Description of Artificial Sequence Synthetic oligonucleotide”
ggaggaauuuguuccugaa

SEQ ID NO: 219 - RNA, 19 nt, Artificial Sequence
  source: /note=“Description of Artificial Sequence Synthetic oligonucleotide”
uucaggaacaaauuccucc

(19) United States
(12) Patent Application Publication
(10) Pub. No.: US 2011/0117627 A1
(43) Pub. Date: May 19, 2011

(54) REGULATION OF APOPTOSIS BY NEURAL SPECIFIC SPLICE VARIANTS OF IG20

(75) Inventors: Bellur S. Prabhakar, Chicago, IL (US); Matthew N. Meriggioli, Chicago, IL (US); Liang Cheng LI, Chicago, IL (US)

(73) Assignee: The Board of Trustees of the University of Illinois, Urbana, IL (US)

(21) Appl. No.: 13/003,215

(22) PCT Filed: Jul. 10, 2009
(86) PCT No.: PCT/US09/50219

§ 371 Date: Jan. 7, 2011

Related U.S. Application Data
(60) Provisional application No. 61/079,739, filed on Jul. 10, 2008.

(51) Int. Cl. C12N 7/01; C07H 21/02; C12N 5/079; C12N 5/10

(52) U.S. Cl. 435/235.1; 536/24.5; 435/325

(57) ABSTRACT

Pro-apoptotic signaling caused by down-modulation of KIAA0358 or expression of IG20-SV4 effectively induces spontaneous apoptosis and sensitization to TNFα-induced apoptosis in neuroblastoma cells. Methods and composition to enhance cell death in neuroblastoma are provided. Methods and compositions to reduce cell death in neurodegenerative disorders are provided.

### CROSS REFERENCE TO RELATED APPLICATIONS

This application claims priority to U.S. provisional patent application No. 61/079,739, filed Jul. 10, 2008, which is herein incorporated by reference in its entirety.

### GOVERNMENT RIGHTS

Part of the work during development of this application was made with government support from the National Institute of Health, NIH (RO1 CA107506); the United States Government has certain rights in the invention.

### BACKGROUND

Methods and compositions are described to regulate apoptosis and caspase-8 expression by isoforms of the IG20 gene.

The IG20 (insulinoma-glucagonoma) gene has been implicated in cancer cell survival and apoptosis, neurotransmission and neurodegeneration. Various splice isoforms of the IG20 gene (IG20-SVs), including IG20pa, MADD/DENN, and DENN-SV, act as negative or positive regulators of apoptosis, and their levels of expression can profoundly affect cell survival in non-neural cells. IG20-SVs are believed to act, in part, by modulating inflammatory and apoptotic signaling pathways, effects mediated through interactions with tumor necrosis factor receptor 1 (TNFR1). TNFα interacts with TNFRI to trigger pro-inflammatory actions through various stress-activated protein kinases (SAPKs), such as c-Jun N-terminal kinase (JNK) and p38 mitogen-activated protein kinase (p38 MAPK). IG20 interacts strongly with TNFR1, and all putative IG20-SVs contain the death domain homology region (DDHR) required for this binding. Expression of MADD/DENN is required and sufficient for cancer cell survival in non-neuronal cancer cells, and mediates its effects by acting as a negative regulator of caspase-8 activation. The over-expression of IG20pa, on the other hand, results in enhanced apoptosis and activation of caspase-8 through enhanced DISC formation. The caspase-8 (CASP8) gene encodes a key enzyme at the top of the apoptotic cascade.

Neuroblastoma (NB) is one of the most frequently occurring solid tumors in children, particularly in the first year of life, when it accounts for 50% of all tumors. Neuroblastoma is a solid, malignant tumor that manifests as a lump or mass in the abdomen or around the spinal cord in the chest, neck, or pelvis. Neuroblastoma is often present at birth, but is most often diagnosed much later when the child begins to show symptoms of the disease. A condition known as “opsoclonus-myoclonus syndrome” can sometimes be a symptom of neuroblastoma. Although improvement in outcome has been observed in small, well-defined subsets of patients over the past several years, the outcome for patients with a high-risk clinical phenotype has not improved, with long-term survival less than 40%. A characteristic feature of NB is its remarkable clinical and biological heterogeneity. While advanced stage NB in older children typically responds poorly to aggressive chemotherapy regimens, certain tumors in patients below one year of age may spontaneously regress or differentiate into benign ganglioneuromas. This spontaneous regression likely represents the activation of an apoptotic and/or differentiation pathway, and the prognosis in NB patients may be related to the level of expression of molecules involved in the regulation of apoptosis.

In NB cell lines and tumor samples, CgG methylation of CASP8 at the 5′ end has been associated with inactivation of the gene, and recent hypotheses have proposed that CASP8 may act as an NB tumor-suppressor gene. Furthermore, NB cell lines that do not express caspase-8 are resistant to TRAIL-induced apoptosis, and suppression of caspase-8 expression has been shown to occur during establishment of NB metastases in vivo.

### SUMMARY

The preferential expression of two unique splice isoforms (KIAA0358, IG20-SV4) of the IG20 gene was demonstrated in selected nervous system tissue and in two neuroblastoma (NB) cell lines known to be deficient in the expression of caspase-8. Through gain-of-function studies, and using siRNA technology, the expression of IG20-SV4 was shown to enhance cellular apoptosis and lead to the expression and activation of caspase-8 in SK-N-SH and SH-SY5Y NB cells, thereby sensitizing these cells to the pro-apoptotic effects of TNFα. In contrast, expression of KIAA0358 effectively rendered cells resistant to apoptosis, even when IG20-SV4 is co-expressed. Down-modulation of this isoform causes markedly enhanced apoptotic cell death and activation of caspase-8.

A composition includes a short-interfering RNA (siRNA) that specifically down regulates the expression of an IG20 splice variant KIAA0358 in a neuroblastoma cell. In an embodiment, the siRNA targets Exon 21 or Exon 26 of the IG20 gene splice transcripts.

In an embodiment, the siRNA comprises a nucleic acid sequence selected from Table 2 that targets Exon 21 or a nucleic acid sequence selected from Table 3 that targets Exon 26 of the IG20 gene.

In an embodiment, the siRNA targets Exon 21 of the IG20 gene in a region that includes or consists essentially of a nucleotide sequence AATTGTGGAACAAGCACCAGGAAGTGAAAAAGCAAAAAGCTTTGGAAAAAC AGA or targets Exon 26 of the IG20 gene in a region that includes or consists essentially of a nucleotide sequence AAGGGACAAAGGATCCATGTGGGACCAGTTAGAGGATGCAGCTATGGAGAC CTTTTCTATAAG.

A composition includes a short-interfering RNA (siRNA) that specifically down regulates the expression of splice variants of IG20 comprising IG20pa, MADD, IG20-SV2, DENN-SV, KIAA0358 except IG20-SV4 in a neuroblastoma cell.

In an embodiment, the siRNA targets Exons 13L and 34 of the IG20 gene. For example, the siRNA targets Exon 13L of the IG20 gene in a region that includes or consists essentially of a nucleotide sequence CGGCGAATCTATGACAATC and targets Exon 34 of the IG20 gene in a region that includes or consists essentially of a nucleotide sequence GGTTTTCATAGAGCTGAATCACATTAAAAAGTGCAATACAGTTCGAGGCGTC TTTGTCCTGGAGGAATTT.

A purified or isolated short-interfering RNA (siRNA) molecule specifically down regulates the expression of an IG20 splice variant KIAA0358 in a neuroblastoma cell. In an embodiment, the siRNA molecule is synthetic and may contain one or more modified residues or analogs to improve stability or bioavailability.

A purified or isolated short-interfering RNA (siRNA) specifically down regulates the expression of splice variants of IG20 comprising IG20pa, MADD, IG20-SV2, DENN-SV, KIAA0358 except IG20-SV4 in a neuroblastoma cell. In an embodiment, the siRNA molecule is synthetic and may contain one or more modified residues or analogs to improve stability or bioavailability.

A purified or isolated vector expresses the siRNA disclosed herein, wherein the siRNA includes a nucleic acid sequence selected from Table 2 that targets Exon 21 or a nucleic acid sequence selected from Table 3 that targets Exon 26.

A purified or isolated vector expresses the siRNA disclosed herein, wherein the siRNA comprises a nucleic acid sequence 5′-AGAGCTGAATCACATTAAA-3′ that targets Exon 13L and includes a nucleic acid sequence 5′-AGAGCTGAATCACATTAAA-3′ that targets Exon 34 of the IG20 gene.

A pharmaceutical composition includes or consists essentially of a short-interfering RNA (siRNA) or a shRNA vector to specifically down regulate an IG20 splice variant KIAA0358 for use as a medicament.

A pharmaceutical composition includes or consists essentially of a short-interfering RNA (siRNA) or a shRNA vector to specifically down regulate an IG20 splice variant KIAA0358 for use to enhance apoptosis in a neuroblastoma cell.

A pharmaceutical composition includes or consists essentially of a short-interfering RNA (siRNA) or a shRNA vector to specifically down regulate an IG20 splice variant KIAA0358 for use in the treatment of neuroblastoma.

A method of increasing cell death in a neuroblastoma includes administering a composition that includes one or more siRNA or a shRNA vector that targets Exon 21 of the IG20 gene in a region including a nucleotide sequence AATTGTGGAACAAGCACCAGGAAGTGAAAAAGCAAAAAGCTTTGGAAAAAC AGA or targets Exon 26 of the IG20 gene in a region including a nucleotide sequence AAGGGACAAAGGATCCATGTGGGACCAGTTAGAGGATGCAGCTATGGAGAC CTTTTCTATAAG. In an embodiment, the cell death is apoptotic.

A method of increasing cell death in a neuroblastoma includes administering a composition that includes one or more siRNA or a shRNA vector, whose sequence includes a nucleic acid sequence selected from the group consisting of nucleotides listed in Table 2 that target Exon 21 or from Table 3 that target Exon 26 or a DNA complement thereof.

A pharmaceutical composition includes or consists essentially of a short-interfering RNA (siRNA) or a shRNA vector to specifically down regulate the expression of splice variants of IG20 including IG20pa, MADD, IG20-SV2, DENN-SV, KIAA0358 except IG20-SV4 for use as a medicament.

A pharmaceutical composition includes or consists essentially of a short-interfering RNA (siRNA) or a shRNA vector to specifically down regulate the expression of splice variants of IG20 including IG20pa, MADD, IG20-SV2, DENN-SV, KIAA0358 except IG20-SV4 for use to enhance apoptosis in a neuroblastoma cell.

A pharmaceutical composition includes or consists essentially of a short-interfering RNA (siRNA) or a shRNA vector to specifically down regulate the expression of splice variants of IG20 including IG20pa, MADD, IG20-SV2, DENN-SV, KIAA0358 except IG20-SV4 for use in the treatment of neuroblastoma. In an embodiment, the siRNA targets Exon 13L and Exon 34 of the IG20 gene.

A method of increasing cell death in a neuroblastoma includes administering a composition that includes one or more siRNA or a shRNA vector, wherein the siRNA targets Exon 34 of the IG20 gene in a region that includes the nucleotide sequence GGTTTTCATAGAGCTGAATCACATTAAAAAGTGCAATACAGTTCGAGGCGTC TTTGTCCTGGAGGAATTT.

A method of increasing cell death in a neuroblastoma includes administering a composition that includes one or more siRNA or a shRNA vector, wherein the siRNA targets Exon 13L of the IG20 gene in a region including a nucleotide sequence CGGCGAATCTATGACAATC and targets Exon 34 of the IG20 gene in a region including a nucleotide sequence GGTTTTCATAGAGCTGAATCACATTAAAAAGTGCAATACAGTTCGAGGCGTC TTTGTCCTGGAGGAATTT.

A method to enhance apoptosis in neuroblastoma cells includes:

(a) specifically down regulating the expression of an IG20 splice variant KIAA0358; or

(b) specifically down regulating the expression of splice variants of IG20 comprising IG20pa, MADD, IG20-SV2, DENN-SV, KIAA0358 except IG20-SV4; or

(c) providing a composition comprising a cDNA sequence for expressing an IG20 splice variant IG20-SV4 or a domain thereof in a neuroblastoma cell.

In an embodiment, the method further includes a TNFα or interferon-γ treatment, wherein the neuroblastoma cells are sensitive to TNFα or interferon-γ treatment. In an embodiment, the method further includes providing a cytotoxic agent in combination or in conjunction with the therapy. Analogs of TNFα including derivatives are suitable.

A method to reduce or rescue cell death to ameliorate one or more conditions associated with a neurodegenerative disorder includes administering a composition comprising a nucleotide sequence coding for KIAA0358 or a coding fragment thereof and expressing the nucleotide sequence or a fragment thereof. The expression of the nucleotide sequence of KIAA0358 or the coding fragment thereof reduces cell death.

In an embodiment, the neurodegenerative disorder is multiple sclerosis or Parkinson's disease.

An engineered mammalian virus includes one or more vectors having one or more siRNA or shRNA sequences disclosed herein. In an embodiment, the vector is adenovirus or adeno-associated virus or lentivirus.

A neural cell transfected with a virus that contains a vector to down regulate KIAA0358 or express KIAA0358 or IG20-SV4. In an embodiment, the neural cell is a neuroblastoma cell or a cell associated with neurodegenerative disorder.

### BRIEF DESCRIPTION OF THE DRAWINGS

FIG. 1. Expression of IG20 splice isoforms in human NB cell lines, primary NB tumor lines, and various human tissues. 1 μg of total RNA was used for reverse transcription-polymerase chain reaction (RT-PCR) using the Super-Script III One-Step RT-PCR system (Invitrogen Life Technologies, Carlsbad, Calif., USA). (A) Shows amplification of exon 34 region of IG20-SVs using F4824 and B5092 primers. (B) Shows quantification of relative intensities of bands in relation to the housekeeping gene GAPDH from panel A using ImageJ (National Institutes of Health, MD, US).

FIG. 2. IG20-SVs and down modulation effect of exon-specific siRNAs directed against specific isoforms on endogenous IG20-SVs in SK-N-SH cells. (A) Shows human IG20-SVs generated by alternative mRNA splicing. Solid bars represent regions of complete cDNA sequence homology between variants. Empty areas indicate spliced exons 13L, 16, 21, 26 and 34, which when spliced in different combinations can give rise to the six IG20-SVs. (B) Effect of down modulation of endogenous IG20-SVs by exon-specific siRNAs in SK-N-SH cells. One microgram total RNA obtained from GFP-positive SK-N-SH cells obtained by fluorescence-activated cell sorting (FACS) at 5 days post-transduction was used for reverse transcription-polymerase chain reaction. The products were separated on a 5% PAGE. Amplification of IG20-SVs using F1-B2 primers (upper panel) and F4824-B5092 (lower panel) is shown. (C) Quantification of relative intensities of bands from panel B (upper panel) using ImageJ. (D) Quantification of relative intensities of bands from panel B (lower panel) using ImageJ.

FIG. 3. Apoptotic effects and caspase-8 activity with down modulation of IG20-SVs in SK-N-SH cells. (A) Representative data showing mitochondrial depolarization as determined by Di1C staining. Five days post-transduction, SK-N-SH cells were collected and one-third cells of the collected cells were stained with 50 nM of DiIC. Loss of staining (as a marker of mitochondrial depolarization) was detected by FACS analysis. Percentage of apoptotic cells are indicated on the histograms. (B) Summary of the results showing percentages of cells with increased mitochondrial depolarization as measured by DiIC staining from three independent experiments. The P-value was **P<0.01, for test groups vs SCR. (C) Summary of results showing percentage of cells with increased apoptosis as determined by Annexin V-PE/7-AAD staining. Another one-third of collected cells as described in (A) were stained with Annexin V-PE/7-AAD and detected by FACS. The P-value was *P<0.05 for test groups vs SCR. The data were gated from GFP-positive cells only. (D) Caspase-8 activity in NB cells transduced with siRNAs. The final one-third of cells from A were lysed and subjected to western blot analysis for caspase-8, caspase-9, and caspase-3. The data shown are representative of three separate experiments.

FIG. 4. Effects of TNF-α treatment on apoptosis of siRNA-transduced SK-N-SH cells. Three days post-transduction, SK-N-SH cells were treated with 10 ng/ml TNF-alpha for 2 days, and cells were collected and stained with Annexin V-PE/7-AAD. (A) Summarized results showing percentage of cells with increased apoptosis from three independent experiments. The P-value was *P<0.05 for TNF-α treated cells vs untreated cells. (B) Summarized results showing percentage of apoptosis in transfected cells treated with TNFα±DN-FADD. Results are from three independent experiments. The P-value was *P<0.05, **P<0.01 for pcDNA-DN-FADD transfected cells vs pcDNA3.1 transfected cells. The data were collected from GFP-positive cells only.

FIG. 5. Expression of KM 0358 in isolation can prevent apoptosis and suppress caspase-8 activity in SK-N-SH cells. (A) RT-PCR of IG20-SVs from stable cells expressing control vector (pEYFP-C1) or YFP-KIAA0358-Mut and infected with Mid-shRNA for five days. (B) Mitochondrial depolarization assay. SK-N-SH cells were stained with DiIC to determine spontaneous apoptosis. Data shown are representative of three independent experiments (**P<0.01 vs SCR, ##P<0.01 vs. Mid+pEYFP-C1). The data were collected from YFP and GFP double-positive cells only. (C) Western blot showing caspase-8 activity. Cell lysates were subjected to western blot analysis of caspase-8. The data shown are representative of three individual experiments.

FIG. 6. Down modulation of KIAA0358 or selective expression of IG20-SV4 enhances apoptosis through expression/activation of caspase-8 in SK-N-SH cells. (A) Effects of cycloheximide on expression/activation of caspase-8. Three days post-transduction with shRNA-expressing virus, SK-N-SH cells were treated with 10 μg/ml cycloheximde (a protein synthesis inhibitor) for two days. Whole cell lysates were subjected to western blot analysis. (B) Caspase-8 reporter assay. SK-N-SH cells were cotransfected with pGL4.17-caspase-8 promoter vector, pSV40-Renilla luciferase vector and pEYFP-C1/or pEYFP-IG20-SV4 using Lipofectamine2000, 48 hrs later, cells were collected and analyzed for luciferase activity with the Dual-Luciferase Reporter Assay System (Promega). (C) and (D) Effects of caspase-8 inhibition. Three days post-transduction with shRNA-expressing virus, SK-N-SH cells were treated with 40 μM and 80 μM of Z-1ETD-FMK (a caspase 8 inhibitor) for two days. Collected cells were either subjected to Annexin V-PE/7-AAD stain for FACS analysis (C) or western blot analysis (D). (C) Percentage apoptosis in cells transduced with different shRNAs in the presence or absence of the caspase inhibitor. The P-value was **P<0.01 for Z-IETD-FMK treated vs untreated. (D) Western blot showing inhibitory effect of Z-IETD-FMK on caspase-8 activity. Representative data are from three independent experiments.

FIG. 7. Effects of down modulation of endogenous IG20-SVs on SK-N-SH cellular proliferation. (A) MTT assay of SK-N-SH cell proliferation, twenty-four-hour post-transduction. Data shown represent mean±SE of analyses performed in three independent experiments. (B) CFSE-red assay for cell proliferation. Twenty-four hours post-transduction, SKNSH cells were stained with CFSE-red (SNARF-1carboxylic acid, acetate, succinimidyl ester), harvested on indicated days and evaluated for CFSE dilution in GFP-positive, gated, SK-N-SH cells by FACS. The numbers on the histograms indicate geometric peak mean intensities of CFSE staining in the transduced cells.

FIG. 8. Apoptotic effects and caspase-8 activity of down modulation of IG20-SVs in SH-SY5Y cells. Five days post-transduction, SH-SY5Y cells were collected and either subjected to Annexin V-PE/7-AAD staining for FACS analysis or were used for western blot analysis. (A) Enhanced apoptosis in SH-SY5Y NB cells transduced with 13L-siRNA and 34E+13L-siRNA. Data shown are a summary of three independent experiments. The P-value was **P<0.01, ***P<0.001 when compared to SCR transduced cells. (B) Western blot analysis of caspase-8. Whole cells lysates was subjected to western blot. The data shown are again representative of three separate experiments.

FIG. 9. Over-expression of IG20-SV4 or KIAA0358 does not affect caspase-8 activity in SK-N-BE(2)-C cells. SK-NBE(2)-C NB cells were transfected with a vector expressing IG20-SV4 or KIAA0358. Forty-eight hours post-transfection, cells were harvested and whole cell lysates were subjected to western blot analysis. No significant increase in expression of full-length or cleaved (p43/p41, p18) caspase-8 was observed as a consequence of over expression.

FIG. 10. Down modulation of KIAA0358 or selective expression of IG20-SV4 induce caspase-8 mRNA expression in SK-N-SH cells. Five days post-transduction with shRNA-expressing virus, RNA was extracted from GFP-positive SK-N-SH cells and used for reverse transcription-polymerase chain reaction. The data shown are representative of three individual experiments.

### DETAILED DESCRIPTION

The insulinoma-glucagonoma (IG20) gene undergoes alternative splicing resulting in the differential expression of six putative splice variants. Four of these (IG20pa, MADD, IG20-SV2 and DENN-SV) are expressed in almost all human tissues. Alternative splicing of the IG20 gene have been largely limited to non-neural malignant and non-malignant cells. The present disclosure provides expression analysis of unique alternative splice isoforms of the IG20 gene was investigated in human neuroblastoma (NB) cells. Six IG20 splice variants (IG20-SVs) were expressed in two human NB cell lines (SK-N-SH and SH-SY5Y), highlighted by the expression of two unique splice isoforms, namely KIAA0358 and IG20-SV4. Similarly, enriched expression of these two IG20-SVs were found in human neural tissues derived from cerebral cortex, hippocampus, and, to a lesser extent, spinal cord. Utilizing gain of function studies and siRNA technology, these “neural-enriched isoforms” were found to exert significant and contrasting effects on vulnerability to apoptosis in NB cells. Specifically, expression of KIAA0358 exerted a potent anti-apoptotic effect in both the SK-N-SH and SH-SY5Y NB cell lines, while expression of IG20-SV4 had pro-apoptotic effects directly related to the activation of caspase-8 in these cells, which have minimal or absent constitutive caspase-8 expression. These data indicate that the pattern of expression of these neural-enriched IG20-SVs regulates the expression and activation of caspase-8 in certain NB cells, and that manipulation of IG20-SV expression pattern represents a potentially potent therapeutic strategy in the therapy of neuroblastoma, and perhaps other cancers.

IG20, MADD, DENN and KIAA0358 are different isoforms of the same gene that stem from alternative splicing of exons 13L, 16, 21, 26 and 34. A total of seven putative IG20-SVs have been identified, namely, IG20pa, MADD, DENN-SV, IG20-SV2, KIAA0358, IG20-SV4, and IG20-FL (Al-Zoubi et al. (2001), J Biol Chem; 276: 47202-11; Efimova et al., (2003), Cancer Res;63(24):8768-8776, the contents of which are herein incorporated by reference).

KIAA0358 and IG20-SV4, which are not highly expressed in non-neural cells, were significantly expressed in cerebral cortex, hippocampus, and to a lesser extent, spinal cord. IG20-SV4 and KIAA0358 were designated as “neural-enriched” IG20-SVs. These neural-enriched isoforms were also found to be expressed in two NB cell lines (SK-N-SH, and SH-SY5Y) known to be deficient in caspase-8 expression, but not in the SK-N-BE(2) NB cell line which is known to express caspase-8. There was relatively little mRNA expression of neural-enriched IG20-SVs in human cerebellum or skeletal muscle. The differential presence of these neural-specific IG20-SVs is consistent with tissue specific differences in alternative splicing of pre-mRNAs.

To investigate the physiological relevance of the expression of the neural-enriched IG20-SVs in NB cells, select combinations of IG20-SVs were down-modulated using siRNAs in SK-N-SH and SH-SY5Y NB cells. Down-modulation of MADD/DENN using shRNA targeting exon 13L enhanced spontaneous apoptosis (SK-N-SH and SH-SY5Y) and TNF-α-induced apoptosis (SK-N-SH) was found. The 13L siRNA will also down-modulate KIAA0358 expression. Down-modulation of all IG20-SVs also resulted in enhanced apoptosis of NB cells in SK-N-SH cells, although not significantly in SH-SY5Y cells. However, selective down-modulation of IG20pa, MADD, IG20-SV2, and DENN-SV, allowing for unaltered endogenous expression of IG20-SV4 and KIAA0358, resulted in markedly enhanced cellular survival in both NB cell lines. In contrast, knock-down of all splice isoforms except for IG20-SV4 caused a significant enhancement of apoptosis in both SK-N-SH and SH-SY5Y cells. These results suggested that KIAA0358 exerts a predominant suppressive effect on IG20-SV4 in certain NB cells. These IG20-SVs (IG20-SV4 and KIAA0358) may be involved in the regulation of caspase-8 activation in NB cells.

Caspase-8 expression was increased in cells in which KIAA0358 was down-modulated (treated with 13L and 34E+13 siRNAs, and, to a lesser extent, in cells in which all IG20-SVs were knocked down). When transduced SK-N-SH cells were treated with cycloheximide, the induced caspase-8 was inhibited, consistent with it being newly synthesized protein, indicating that the pattern of IG20-SV4 and KIAA0358 expression may be involved in the regulation of CASP8 gene expression. This was confirmed by showing the effect of IG20-SV4 on activation of the CASP8 promoter utilizing a luciferase assay. The marked activation of the CASP8 promoter by IG20-SV4 is direct evidence that IG20-SVs may exert their effects through regulation of CASP8 gene expression. Inhibition of caspase-8 protected cells from undergoing apoptosis only when KIAA0358 was down-modulated, i.e., utilizing 13L, 34E+13L and mid siRNAs.

The mechanism of enhanced apoptosis in these cells likely was related to caspase-8 expression and activation. Furthermore, the selective expression of IG20-SV4 sensitized NB cells to the pro-apoptotic effects of TNFα, and this sensitization was suppressed by DN-FADD, offer further support for the mechanistic role of caspase-8 in enhancement of both spontaneous and TNFα-induced apoptosis mediated by selective overexpression of IG20-SV4.

While levels of apoptosis and caspase-8 activation were very high in NB cells in which all IG20-SVs except IG20-SV4 were down-modulated, selective expression of KIAA0358 in the presence of IG20-SV4 (or in the setting of down-modulation of all other isoforms) effectively prevented apoptosis and caspase-8 expression, indicating that KIAA0358 may have a dominant-negative effect on IG20-SV4. To further confirm the pro-survival effects of KIAA0358 on NB cell survival, SK-N-SH cells stably expressing a mutant KIAA0358 were generated which contained silent mutations that did not affect protein expression, but prevented down-modulation of KIAA0358 by mid-shRNA. The cell was transduced with MID-shRNA for 5 days. SK-N-SH cell lines expressing this KIAA0358 mutant were largely resistant to apoptosis compared to control cells treated with mid-shRNA. This effect was accompanied by a nearly complete dampening of caspase-8 activation. While the effects of manipulation of neural IG20-SVs were similar in the SK-N-SH and SH-SY5Y cell-lines (both deficient in caspase-8), no effect of introduction of either IG20-SV4 or KIAA0358 on caspase-8 expression was observed in the SK-N-BE(2)-C cell line which has constitutive expression of caspase-8.

Silencing of the CASP8 gene may play a role in NB tumor progression by the induction of tumor cell resistance to apoptosis induced by cytotoxic agents, or by death-inducing ligands, such as TNF-α or TRAIL. Further, interferon-γ can sensitize neoplastic cells to apoptosis through up-regulation of caspase-8, and an interferon-sensitive response element (ISRE) in the caspase-8 promoter may play a role in this IFN-γ-driven regulation of caspase-8 expression in cancer cells. The regulation of caspase-8 expression likely involves other complex interactions involving the CASP8 gene. Expression of IG20-SVs may play a role in determining caspase-8 expression/activation and susceptibility to apoptosis in NB cells.

Pro-apoptotic signaling caused by down-modulation of KIAA0358 or overexpression of IG20-SV4 effectively induces spontaneous apoptosis and sensitization to TNFα-induced apoptosis through expression and activation of caspase-8 in NB cells known to be deficient in caspase-8. Furthermore, enhanced expression of IG20-SV4 alone can overcome the transcriptional inhibition of the CASP8 gene, and upregulate its expression, while KIAA0358 acts as a negative regulator of caspase-8 expression and activation in these cells. Novel targets that can be manipulated to enhance apoptosis (both spontaneous and in response to cytotoxic drugs) in cancer cells, are developed using the materials and methods described herein.

Neuroblastoma is a solid tumor that most often initiates in one of the adrenal glands, but can also form in nerve tissues in the neck, chest, abdomen, or pelvis. Neuroblastoma may be classified into three risk categories: low, intermediate, and high risk. About 60% of all neuroblastoma cases exhibit metastases. Multimodal therapy (e.g., chemotherapy, surgery, radiation therapy, stem cell transplant, and immunotherapy (e.g., with anti-GD2 monoclonal antibody therapy) can also be administered in combination or in conjunction with the methods and compositions disclosed herein that down regulate one or more splice variants of IG20. Chemotherapy agents used in combination have been found to be effective against neuroblastoma. Refractory and relapsed neuroblastoma are also capable of being treated with the compositions disclosed herein.

The term “splice variants” as used herein refer to the various RNA transcripts of the IG20 gene produced by alternative splicing by which the exons of the RNA produced by transcription of the IG20 gene (a primary gene transcript or pre-mRNA) are reconnected in multiple ways during RNA splicing. The resulting different mRNAs may be translated into different protein isoforms (splice variants); thus, a single gene may code for multiple proteins or polypeptides. These include IG20pa, MADD, IG20-SV2, DENN-SV, IG20-SV4 and KIAA0358 or partial fragments thereof including those containing SNPs or naturally occurring variants thereof.

RNA interference (RNAi) is the pathway by which short interfering RNA (siRNA) or short hairpin RNA (shRNA) are used to downregulate the expression of target genes. Synthetic small interfering (siRNAs) or expressed stem-loop RNAs (short-hairpin RNAs (shRNAs) or artificial microRNAs (miRNAs) have been delivered to cells and organisms to inhibit expression of a variety of genes. Such RNA molecules form hairpin-shaped double-stranded RNA (dsRNA) Nucleic acid molecules for shRNA are cloned into a vector under a suitable promoter, for example, a pol III type promoter. Expressed shRNA is transcribed in cells from a DNA template as a single-stranded RNA molecule (˜50-100 bases). Complementary regions spaced by a small ‘loop’ or ‘intervening’ sequence result in the formation of a ‘short hairpin’. Cellular recognition and processing by the RNAi machinery converts the shRNA into the corresponding siRNA. Exemplary design methodologies for producing shRNA templates is found in McIntyre and Fanning, BMC Biotechnology 2006 6:1.

The term “short interfering nucleic acid”, “siRNA”, “short interfering RNA”, “short interfering nucleic acid molecule”, “short interfering oligonucleotide molecule”, or “chemically-modified short interfering nucleic acid molecule” as used herein refers to any nucleic acid molecule capable of reducing or down regulating gene expression, for example, through RNA interference “RNAi” or gene silencing in a sequence-specific fashion.

The present disclosure provides an expression cassette containing an isolated nucleic acid sequence encoding a small interfering RNA molecule (siRNA) targeted against one or more splice variants of the IG20 gene. The shRNA expression cassette may be contained in a viral vector. An appropriate viral vector for use herein invention may be an adenoviral, lentiviral, adeno-associated viral (AAV), poliovirus, herpes simplex virus (HSV), Picornavirus, or murine Maloney-based viral vector. In an embodiment of the present invention, siRNA in a brain cell or brain tissue is generated. A suitable vector for this application is an FIV vector (Brooks et al. (2002), Proc. Natl. Acad. Sci. U.S.A. 99:6216-6221; Alisky et al., NeuroReport. 11, 2669 (2000a) or an AAV vector. For example, AAV5 vector is useful (Davidson et al. (2000), Proc. Natl. Acad. Sci. U.S.A. 97:3428-3432 (2000). Also, poliovirus or HSV vectors are useful. (Alisky et al., Hum Gen Ther, 11, 2315 (2000)).

Synthetic RNA, recombinantly produced RNA, as well as altered RNA that differs from naturally occurring RNA by the addition, deletion, substitution and/or alteration of one or more nucleotides are within the scope of this disclosure. Nucleotides in the RNA molecules of the instant disclosure may include non-standard nucleotides, such as non-naturally occurring nucleotides or chemically synthesized nucleotides or deoxynucleotides. These altered RNAs are referred to as analogs or analogs of naturally-occurring RNA. The dsRNA molecules (e.g., siRNA and shRNA) of the invention can include naturally occurring nucleotides or include one or more modified nucleotides, such as a 2′-O-methyl modified nucleotide, a nucleotide comprising a 5′-phosphorothioate group, and a terminal nucleotide linked to a cholesteryl derivative or dodecanoic acid bisdecylamide group. Chemically modified double stranded nucleic acid molecules that mediate RNA interference are described in e.g., US20060217331. Chemical modifications of the siRNA molecules may enhance stability, nuclease resistance, activity, and/or bioavailability.

The terms “heterologous gene”, “heterologous DNA sequence”, “exogenous DNA sequence”, “heterologous RNA sequence”, “exogenous RNA sequence” or “heterologous nucleic acid” each refer to a nucleic acid sequence that either originates from a source different than the particular host cell, or is from the same source but is modified from its original or native form.

A subject can be a mammal or mammalian cells, including a human or human cells or human cancer cells.

As used herein, “target sequence” refers to a contiguous portion of the nucleotide sequence of an mRNA molecule formed during the transcription of one or more splice variants of the IG20 gene, including mRNA that is a product of RNA processing of a primary transcription product. By “gene”, or “target gene”, is meant a nucleic acid that encodes a RNA, for example, nucleic acid sequences including, but not limited to, one or more splice variants of the IG20 gene. A gene or target gene can also encode a functional RNA (fRNA) or non-coding RNA (ncRNA), such as small temporal RNA (stRNA), micro RNA (miRNA), small nuclear RNA (snRNA), short interfering RNA (siRNA), small nucleolar RNA (snRNA), ribosomal RNA (rRNA), transfer RNA (tRNA) and precursor RNAs thereof. Such non-coding RNAs can serve as target nucleic acid molecules for siRNA mediated RNA interference in modulating the activity of FRNA or ncRNA involved in functional or regulatory cellular processes.

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5.times.Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C. followed by a high-stringency wash consisting of 0.1×SSC containing EDTA at 55° C.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide which is substantially complementary to a contiguous portion of the mRNA of interest (e.g., one or more splice variants of the IG20 gene). For example, a polynucleotide is complementary to at least a part of one or more splice variants of the IG20 mRNA if the sequence is substantially complementary to a non-interrupted portion of a mRNA encoding splice variant.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

By “asymmetric duplex” as used herein is meant a siRNA molecule having two separate strands that includes a sense region and an antisense region of varying lengths. An antisense region has length sufficient to mediate RNAi in a cell or in vitro system (e.g. about 15 to about 30, or about 15, 16, 17, 18, 19, 20, 21, 22, 23, 24, 25, 26, 27, 28, 29, or 30 nucleotides) and a sense region has about 10 to about 25 (e.g., about 9, 10, 11, 12, 13, 14, 15, 16, 17, 18, 19, 20, 21, 22, 23, 24, or 25) nucleotides that are complementary to the antisense region.

“Introducing into a cell”, or “administering” refers to uptake or absorption into the cell, as is understood by those skilled in the art including passive diffusion or mediated by active cellular processes.

The term “modulate” is means that the expression of the gene, or level of RNA molecule or equivalent RNA molecules encoding one or more splice variants of IG20, is up regulated or down regulated, such that expression, level, or activity is greater than or less than that observed in the absence of the modulator, e.g., a siRNA.

By “inhibit”, “down-regulate”, or “reduce”, it is meant that the expression of the gene, or level of RNA molecules or equivalent RNA molecules encoding one or more proteins or protein subunits or splice variants of the IG20 gene, or activity of one or more proteins or protein subunits, is at least partially reduced or suppressed to below that observed in the absence of a modulator (e.g., siRNA) of the invention. The terms “silence”, “down regulate” and “inhibit”, in as far as they refer to the expression of one or more splice variants of the IG20 gene, refer to the at least partial suppression of the expression of the one or more splice variants of the IG20 gene, as evidenced by a reduction of the amount of mRNA transcribed from the one or more splice variants of the IG20 gene. Alternatively, the degree of inhibition may be given in terms of a reduction of a parameter that is functionally linked to IG20 splice variant transcription, e.g. the amount of protein encoded by the one or more splice variants of the IG20 gene, or the number of cells displaying a certain phenotype, e.g., apoptosis. The degree of inhibition can be greater than 50%, 60%, 75%, 80%, 90%, 95%, and 99%. For example, in certain instances, expression of the one or more splice variants of the IG20 gene is suppressed by at least about 20%, 25%, 35%, or 50% by administration of the RNAi agents disclosed herein. The term “specifically” in the context of “down regulate” refers to a substantially specific suppression of a particular IG20 splice variant.

The terms “level of expression” or “expression level” in are used generally refer to the amount of a polynucleotide or an amino acid product or protein in a biological sample.

The term “treatment” or “therapeutics” refers to the application or administration of a therapeutic agent to a patient, or application or administration of a therapeutic agent to an isolated tissue or cell line from a patient, who has a disorder, e.g., a disease or condition (e.g., neuroblastoma), a symptom of disease (e.g., a neurodegenerative disorder), or a predisposition toward a disease, to cure, heal, alleviate, relieve, alter, remedy, ameliorate, improve, or affect the disease or the symptoms of disease or condition. Treatment can refer to the reduction of any symptom associated with cancer including extending the survival rate of an individual.

As used herein, the phrases “therapeutically effective amount” and “prophylactically effective amount” refer to an amount that provides a therapeutic benefit in the treatment, prevention, or management of the disease or condition. e.g., symptom of neuroblastoma. The specific amount that is therapeutically effective can be readily determined by ordinary medical practitioner, and may vary depending on factors known in the art, such as, e.g. the stage of the cancer, patient's age and other medical history.

As used herein, a “pharmaceutical composition” comprises a pharmacologically effective amount of an RNAi agent or a viral vector or a polypeptide or protein and a pharmaceutically acceptable carrier. As used herein, “pharmacologically effective amount,” “therapeutically effective amount” or simply “effective amount” refers to that amount of nucleic acid or protein/polypeptide effective to produce the intended pharmacological, therapeutic or preventive result.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium.

As used herein, a “transformed cell” or “transfected cell” is a cell into which a vector has been introduced from which a dsRNA molecule (e.g., shRNA) may be expressed.

In one embodiment, the siRNA molecules of the invention are used to treat cancer or other proliferative diseases, disorders, and/or conditions in a subject or organism.

By “cancer” or “proliferative disease” is meant, any disease characterized by unregulated cell growth or replication as is known in the art; brain cancers such as meningiomas, glioblastomas, lower-grade astrocytomas, oligodendrocytomas, pituitary tumors, schwannomas, and metastatic brain cancers; and other proliferative diseases that can respond to the modulation of disease related gene (e.g., “IG20 neural splice variants”) expression in a cell or tissue, alone or in combination with other therapies.

In one embodiment, the disclosure provides double-stranded ribonucleic acid (dsRNA) molecules for inhibiting the expression of the one or more splice variants of the IG20 gene in a cell or mammal, wherein the dsRNA. The dsRNA can be synthesized by standard methods known in the art, e.g., by use of an automated DNA synthesizer that are commercially available.

The dsRNA can contain one or more mismatches to the target sequence. In a preferred embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. Consideration of the efficacy of dsRNAs with mismatches in inhibiting expression of one or more splice variants of the IG20 gene is recognized, especially if the particular region of complementarity in the one or more splice variants of the IG20 gene is known to have polymorphic sequence variation within the population.

A siRNA or shRNA molecule can include any contiguous IG20 splice variant sequence that are variant specific (e.g., about 15 to about 25 or more, or about 15, 16, 17, 18, 19, 20, 21, 22, 23, 24, or 25 or more contiguous IG20 gene nucleotides).

In an embodiment, nucleic acid molecules that act as mediators of the RNA interference gene silencing response are double-stranded nucleic acid molecules. In another embodiment, the siRNA or shRNA molecules include duplex nucleic acid molecules containing about 15 to about 30 base pairs between oligonucleotides having about 15 to about 30 (e.g., about 15, 16, 17, 18, 19, 20, 21, 22, 23, 24, 25, 26, 27, 28, 29, or 30) nucleotides. In another embodiment, siRNA or shRNA molecules include duplex nucleic acid molecules with overhanging ends of about 1 to about 3 (e.g., about 1, 2, or 3) nucleotides, for example, about 21-nucleotide duplexes with about 19 base pairs and 3′-terminal mononucleotide, dinucleotide, or trinucleotide overhangs.

In an embodiment, the siRNA molecules that target one or more splice variants of the IG20 gene are added directly, or can be complexed with cationic lipids, e.g., packaged within liposomes, or otherwise delivered to target cells or tissues. The nucleic acid or nucleic acid complexes can be locally administered to relevant tissues ex vivo, or in vivo through direct application, or injection, with or without their incorporation in biopolymers.

In another aspect, the invention provides mammalian cells containing one or more siRNA or shRNA molecules of this invention. The one or more siRNA or shRNA molecules can independently be targeted to the same or different sites.

The nucleic acid molecules of the instant invention, individually, or in combination or in conjunction with other drugs or agents, can be used to for preventing or treating cancer or proliferative diseases and conditions in a subject or organism. For example, DNA damaging agents such as, doxorubicin, irinotecan, cyclophosphamide, chlorambucil, melphalan, methotrexate, cytarabine, fludarabine, 6-mercaptopurine, 5-fluorouracil, cisplatin, carboplatin, oxaliplatin, and a combination thereof can be used in conjunction or in combination with one or more compositions or treatments disclosed herein. For example, a siRNA therapy to down modulate one or more splice variants of the IG20 gene can be combined with a cytotoxicity therapy for cancers.

Suitable chemotherapy agents include for example, Cyclophosphamide (CYTOXAN™), Chlorambucil (LEUKERAN™), Melphalan (ALKERAN™) Methotrexate (RHEUMATREX™), Cytarabine (CYTOSAR-U™), Fludarabine (FLUDARA™), 6-Mercaptopurine (PURINETHOL™), 5-Fluorouracil (ADRUCIL™) Vincristine (ONCOVIN™), Paclitaxel (TAXOL™), Vinorelbine (NAVELBINE™), Docetal, Abraxane, Doxorubicin (ADRIAMYCIN™), Irinotecan (CAMPTOSAR™), Cisplatin (PLATINOL™), Carboplatin (PARAPLATIN™), Oxaliplatin, Tamoxifen (NOLVADEX™), Bicalutamide (CASODEX™), Anastrozole (ARIMIDEX™), Examestane, Letrozole, Imatinib (GLEEVEC™), Gefitinib, Erlotinib, Rituximab (RITUXAN™), Trastuzumab (HERCEPTIN™), Gemtuzumab, ozogamicin, Interferon-alpha, Tretinoin (RETIN-A™, AVITA™, RENOVA™), Arsenic trioxide, Bevicizumab (AVASTIN™), bortezombi (VELCADE™), cetuximab (ERBITUX™), erlotinib (TARCEVA™), gefitinib (IRESSA™), gemcitabine (GEMZAR™), lenalidomide (REVLIMID™), Serafinib, Sunitinib (SUTENT™), panitumumab (VECTIBIX™), pegaspargase (ONCASPAR™), and Tositumomab (BEXXAR™).

For example, the siRNA or shRNA molecules can be administered to a subject or can be administered to other appropriate cells evident to those skilled in the art, individually or in combination with one or more drugs under conditions suitable for the treatment.

In a further embodiment, the siRNA or shRNA molecules can be used in combination with other known treatments to prevent or treat cancer, proliferative, or other diseases and conditions in a subject or organism.

In one embodiment, a siRNA or shRNA molecule is complexed with delivery systems as described in U.S. Patent Application Publication No. 2003077829 and International PCT Publication No. WO 02/087541, incorporated by reference herein to the extent that they relate to delivery systems.

In one embodiment, siRNA or shRNA or miRNA molecules are administered to a subject by systemic administration in a pharmaceutically acceptable composition or formulation. By “systemic administration” is meant in vivo systemic absorption or accumulation of drugs in the blood stream followed by distribution throughout the entire body. Administration routes that lead to systemic absorption include, without limitation: intracranial, intravenous, subcutaneous, intraperitoneal, inhalation, oral, intrapulmonary, intramuscular, and direct injection to tumor sites. Each of these administration routes exposes the siRNA or shRNA or miRNA molecules to an accessible diseased tissue. The rate of entry of a drug into the circulation has been shown to be a function of molecular weight or size. The use of a liposome or other drug carrier that includes the compounds disclosed herein can potentially localize the drug, for example, in certain tissue types, neural tissues. In addition, delivery systems that specifically aid in increasing the transport of the compositions disclosed herein across the blood brain barrier are also suitable. Examples include Angiopep (AngioChem, Inc., Montreal, Calif.) that modulate uptake bypassing the blood brain barrier by influencing the surface receptors within the blood brain barrier. To deliver the vector specifically to a particular region of the central nervous system, especially to a particular region of the brain, it may be administered by sterotaxic microinjection. Additional routes of administration may be used, e.g., superficial cortical application under direct visualization, or other non-stereotaxic application.

In an embodiment, cationic liposomes conjugated with monoclonal antibodies (immuno liposomes) directed against the disialoganglioside GD2 (antigen on malignant cells) are used as delivery vehicles to deliver the compositions disclosed herein to ameliorate one or more symptoms associated with neuroblastoma. In general, some of the methodologies to deliver siRNA include liposomes—siRNA is encapsulated in lipid vesicles; polyplexes—a cationic carrier binds siRNA to form siRNA-containing nanoparticles; liposome-polycation-nucleic acid complexes—an siRNA-containing polyplex that is encapsulated in a lipid vesicle; and siRNA derivatives—siRNA is conjugated to a targeting group that targets the siRNA into the cells via receptor-mediated endocytosis. See Shen Y (2008), IDrugs;11(8):572-8 (Review).

By “pharmaceutically acceptable formulation” or “pharmaceutically acceptable composition” is meant, a composition or formulation that allows for the effective distribution of the nucleic acid molecules for their desired activity.

A pharmaceutically effective dose is that dose required to prevent, inhibit the occurrence, or treat (alleviate a symptom to some extent, preferably all of the symptoms) of a disease state, e.g., neuroblastoma or a neurodegenerative disorder. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

“Gene delivery,” “gene transfer,” “nucleic acid transfer”, or “siRNA transport” refer to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection or various other protein-based or lipid-based gene delivery complexes) as well as other suitable techniques facilitating the delivery of “naked” polynucleotides.

A “nucleic acid delivery system” refers to any molecule(s) that can carry inserted polynucleotides into a host cell. Examples include liposomes, biocompatible polymers, including natural polymers and synthetic polymers; lipoproteins; polypeptides; polysaccharides; lipopolysaccharides; artificial viral envelopes; recombinant yeast cells, metal particles; and bacteria or viruses, such as baculovirus, adenovirus and retrovirus, bacteriophage, cosmid, plasmid, fungal vectors, nanoparticles, and other recombination vehicles used for biological therapeutics.

A “viral vector” refers to a recombinantly produced virus or viral particle that includes a polynucleotide to be delivered into a host cell, optionally either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, are also useful. See, Schlesinger and Dubensky (1999) Curr. Opin. Biotechnol. 5:434-439 and Ying et al. (1999) Nat. Med. 5(7):823-827. In a particular embodiment, the viral vector is selected from the group consisting of adenovirus, adeno associated virus (MV), vaccinia, herpesvirus, baculovirus and retrovirus.

The terms “adenovirus (Ad) or adeno-associated virus (AAV)” refer to a vector construct that includes the viral genome or part thereof of an adeno virus or an adeno associated virus and a transgene. Adenoviruses (Ads) are a relatively well characterized, homogenous group of viruses, including over 50 serotypes. Recombinant Ad derived vectors are also suitable and known in the art.

As used herein, the terms “treating,” “treatment”, or “therapy” refer to obtaining a desired therapeutic, pharmacologic and/or physiologic effect of the disease or condition treated. The effect may be prophylactic, i.e., a substantially complete or partial prevention of the disease or a sign or symptom thereof, and/or may be therapeutic, i.e., a partial or complete cure for the disorder and/or adverse effect attributable to the disorder. As used herein, to “treat” further includes systemic amelioration of the symptoms associated with the pathology and/or a delay in onset of symptoms.

Intracranial administration may be at any region in the brain and may encompass multiple regions when more than one intracranial delivery is administered. Such sites include, for example, in the brainstem (medulla and pons), mesencephalon, midbrain, cerebellum (including the deep cerebellar nuclei), diencephalon (thalamus, hypothalamus), telencephalon (corpus striatum, midbrain, cerebral cortex, or, within the cortex, the occipital, temporal, parietal or frontal lobes).

The compositions as disclosed herein may further comprise at least a first liposome, lipid, lipid complex, microsphere, microparticle, nanosphere, or nanoparticle, as may be desirable to facilitate or improve delivery of the therapeuticum to one or more cell types, tissues, or organs in the animal to be treated.

“Neurological disease” and “neurological disorder” refer to both hereditary and sporadic conditions that are characterized by nervous system dysfunction, and which may be associated with atrophy of the affected central or peripheral nervous system structures, or loss of function without atrophy. A neurological disease or disorder that results in atrophy is commonly called a “neurodegenerative disease” or “neurodegenerative disorder.” Neurodegenerative diseases and disorders include, but are not limited to, amyotrophic lateral sclerosis (ALS), hereditary spastic hemiplegia, primary lateral sclerosis, spinal muscular atrophy, Kennedy's disease, Alzheimer's disease, Parkinson's disease, multiple sclerosis, and repeat expansion neurodegenerative diseases, e.g., diseases associated with expansions of trinucleotide repeats such as polyglutamine (polyQ) repeat diseases, e.g., Huntington's disease (HD), spinocerebellar ataxia (SCA1, SCA2, SCA3, SCA6, SCAT, and SCA17), spinal and bulbar muscular atrophy (SBMA), dentatorubropallidoluysian atrophy (DRPLA).

The term “consisting essentially of” refers to compositions that contain siRNA or shRNA or miRNA and may optionally contain any other components that do not materially affect the functional attributes of siRNA or shRNA or miRNA. As this refers to the nucleic acid sequences, any sequence that does not materially affect the desired function (e.g., down regulation of one or more splice variants of the IG20 gene or over expression of one or more splice variants or fragments thereof of the IG20 gene), is within the scope of the nucleic acid molecules.

### EXAMPLES

The following examples are for illustrative purposes and are not intended to limit the scope of the pending claims.

### Example 1

### Expression of IG20 Splice Variants in Neuroblastoma Cell Lines and Nervous System Tissues

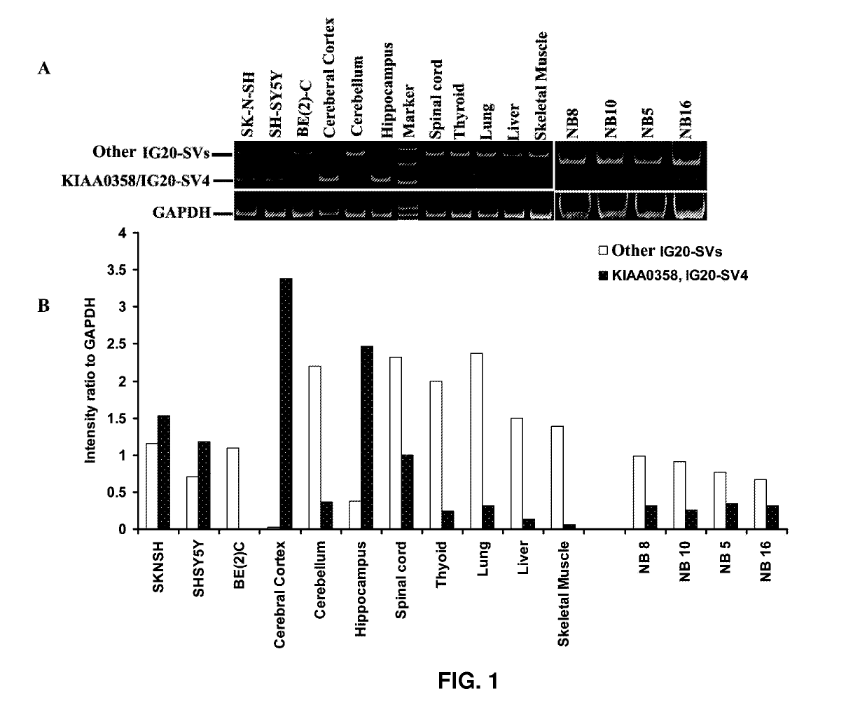

To examine the relevance of IG20 alternative splicing in the control of apoptosis in NB cells, the constitutive expression patterns of IG20-SVs were tested in several NB cell culture lines. RNA extracted from the SK-N-SH, SH-SY5Y, and SK-N-BE(2)-C human NB cell lines was used. RT-PCR was performed using multiple sets of IG20-specific primers as described in the Materials and Methods section. FIG. 1 shows the expression pattern of IG20-SVs in the tested tissues and cell lines.

Although only one representative sample for each tissue type is shown, RNAs from multiple samples of each tissue type were used to validate the RT-PCR results. Different IG20 splice variants are expressed in different patterns and levels in various human tissues. In addition, two isoforms, KIAA0358 and IG20-SV4, were found which are not significantly expressed in non-neural tissues, are highly expressed in two of the three human NB cell lines (SK-N-SH and SH-SY5Y) tested, and in human cerebral cortex, hippocampus, and, to a lesser extent, spinal cord (FIG. 1). In addition, these two isoforms were expressed in both caspase 8-expressing (NB5, NB 16) and caspase 8-deficient (NB8, NBI O) primary NB tumor lines. The levels of expression of KIAA-0358 and IG20-SV4 did not correlate with constitutive expression of caspase-8 in these cells.

### Example 2

### Small Inhibitory RNAs Effectively Down-Modulate Expression of Endogenous IG20-SVs in Neuroblastoma Cells

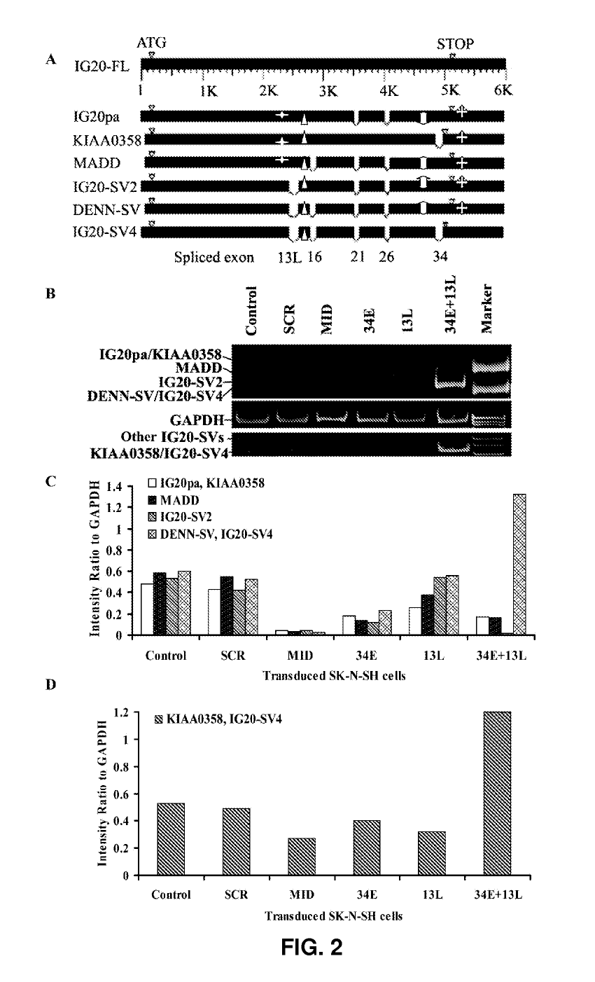

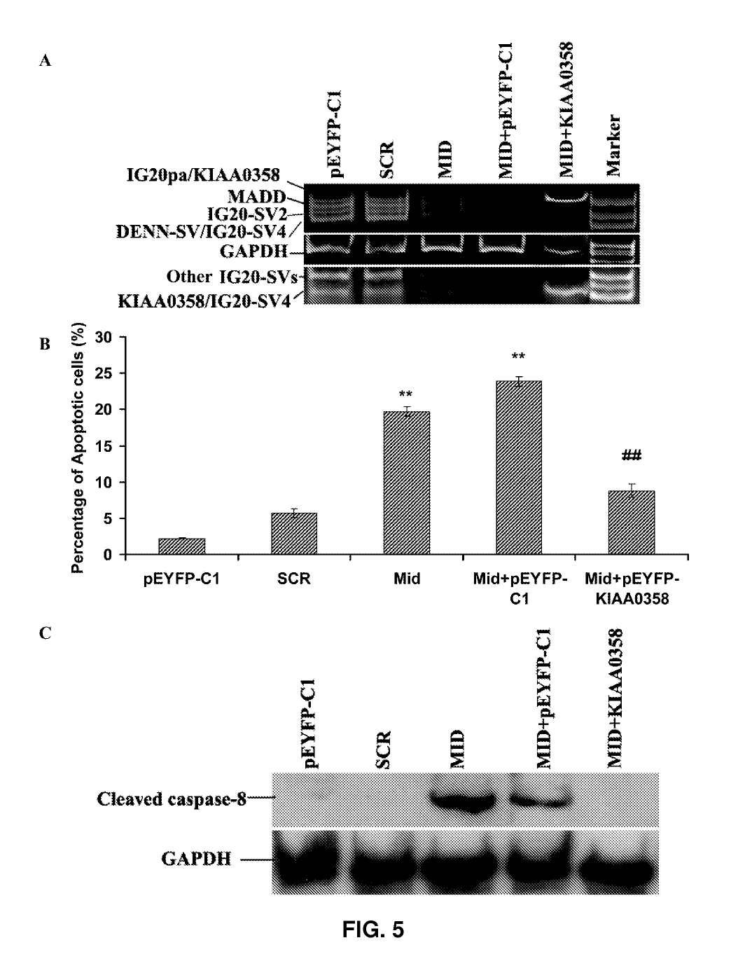

To analyze the effects of IG20-SVs on NB cell survival and apoptosis, small inhibitory RNAs (siRNAs) were designed to selectively down-modulate specific IG20-SVs as shown in FIG. 2A and FIG. 5. The most effective siRNAs targeting all isoforms and targeting exons 13L were identified in studies using Hela cells and PA-1 cells. Several siRNAs targeting exon 34 were screened and the most effective used. Each siRNA was cloned in lentiviral vectors to allow for stable expression of the siRNAs that could be detected through GFP expression.

The targeted exons and resulting down-modulated IG20 isoforms for each siRNA used are summarized in FIG. 2A and Table 1. shRNAs were cloned into a self-inactivating lentivirus vector (pNL-SIN-GFP) (Cullen et al., 2005) and generated 13L, Mid-, 34E and SCR (negative control shRNA) constructs. Utilizing GFP, this enabled monitoring expression of double copy cassettes likely resulting in enhanced silencing. The transduction efficiency was greater than 50% as determined by GFP expression. For testing the down-modulation efficiency, total RNA from transduced and GFP-positive SK-N-SH cells was used for RT-PCR. The results are shown in FIGS. 2B-2D. SK-N-SH cells expressing Mid-shRNA showed decreased expression levels of all IG20-SVs relative to control (SCR). 13L-shRNA caused down-modulation of IG20pa, MADD, and KIAA0358. 34E-shRNA caused down-modulation of IG20pa, MADD, IG20-SV2, and DENN-SV; and 34E+13L-shRNA caused down-modulation of all of these IG20-SVs with the addition of KIAA0358. When all isoforms except IG20-SV4 were down-modulated, expression of this sole isoform appeared to be increased at five days post-transduction (FIGS. 2B, C, D).

### Example 3

### Down-Modulation of KL4A0358 in Neuroblastoma Cells Leads to Spontaneous Apoptosis, but has no Apparent Effect on Cellular Proliferation

a. Down-Modulation of IG20-SVs has no Effect on Cellular Proliferation of NB Cells.

In order to assess the influence of IG20-SVs on NB cell growth and proliferation, various shRNA-expressing viable cells were counted using a MTT assay and CFSE dilution. Relative to controls, a significant decrease in the numbers of viable cells expressing Mid-, 34E , 13L and 34E+13L shRNA was observed (FIG. 1). However, there was no difference in CFSE dilution (SNARF-1 carboxylic acid, acetate, succinimidyl ester) over time amongst the SCR control, Mid-, 34 and 13L and 34+13L-shRNA-treated cells suggesting that the differences in cell numbers were not due to decreased cellular proliferation (FIG. 1B). Further, shRNA-treated cells failed to show significant differences in cell cycle progression. Together, these results indicated that manipulation of the expression patterns of IG20-SVs had little or no effect on cell proliferation or cell cycle progression.

b. Down-Modulation of KIAA0358 Induces Apoptosis in SK-N-SH NB Cells.

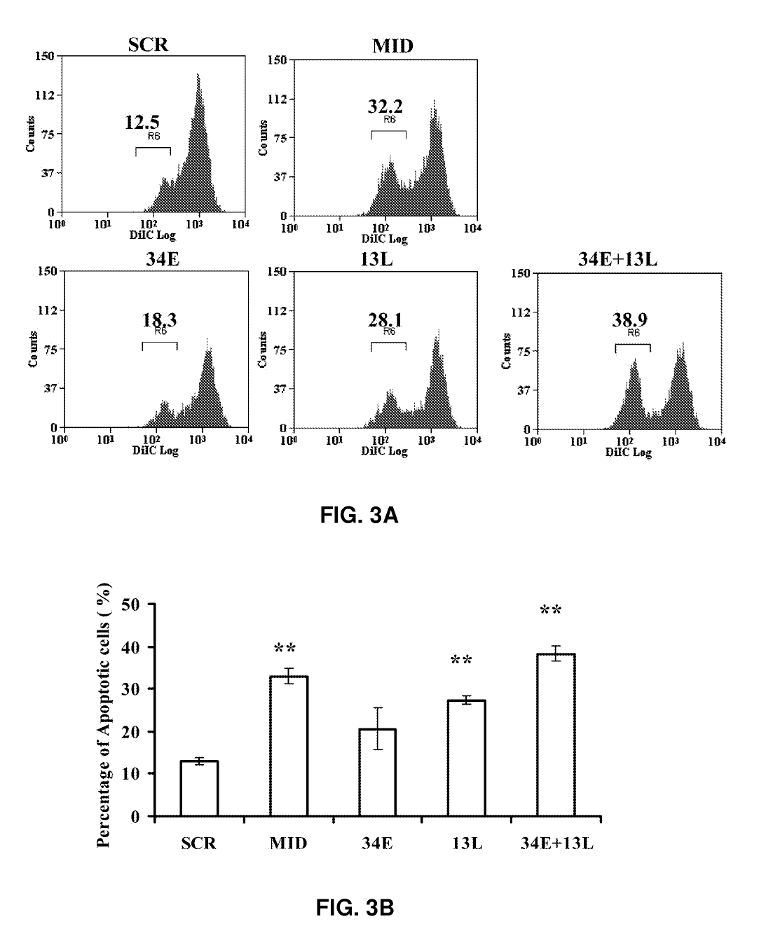

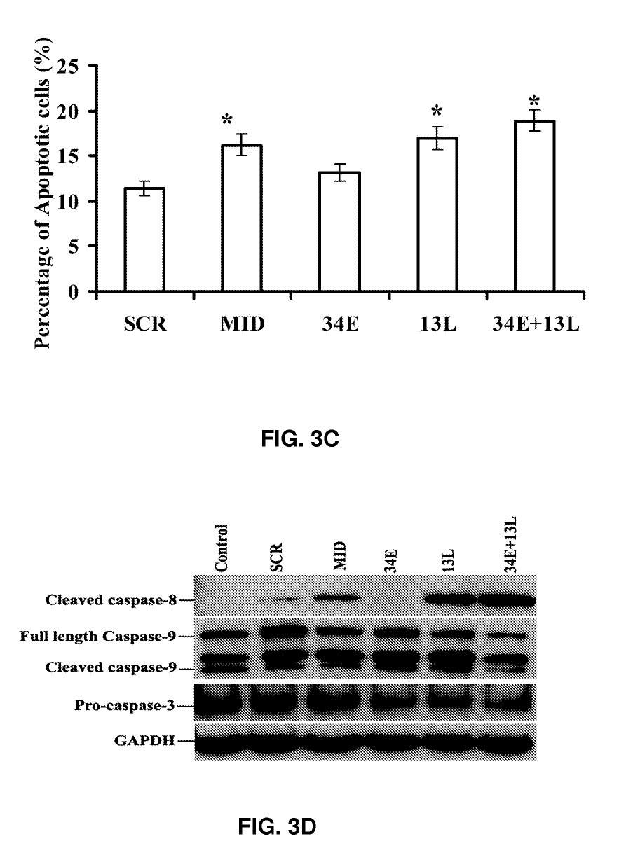

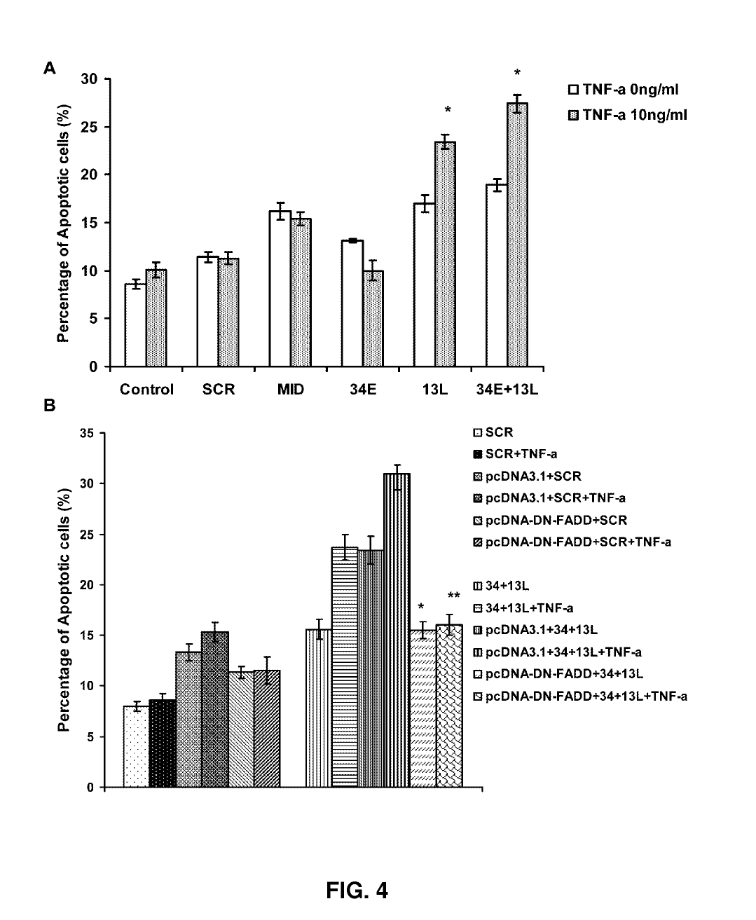

Since there is no single method that can conclusively demonstrate cellular apoptosis, spontaneous cell death was determined using both mitochondrial membrane potential DiIC staining (FIGS. 3A and 3B) and Annexin V-PE/7-AAD staining (FIG. 3C) to assure the reliability of findings. Down-modulation of all IG20-SVs with Mid-shRNA resulted in a significant increase in spontaneous apoptosis. Down-modulation of IG20pa, MADD, KIAA0358 (by targeting exon 13L) and down-modulation of all isoforms with the exception of IG20-SV4 (by targeting exons 13L and 34E) also resulted in significantly increased spontaneous apoptosis. These results were consistently observed using both methods of apoptosis determination and after repeating all experiments a minimum of three times. This suggested that certain IG20-SVs may act as pro-survival factors since their knock down resulted in spontaneous apoptosis. Candidates for this pro-survival function were MADD/DENN and KIAA0358 based on the pro-apoptotic results of down-modulation of these two IG20-SVs. The selective expression of KIAA0358 and IG20-SV4 in the absence of other isoforms (targeting exon 34) resulted in markedly reduced apoptotosis. This finding strongly indicated that expression of KIAA0358 had a pronounced anti-apoptotic effect, since expression of IG20-SV4 alone (in the absence of all other isoforms including KIAA0358) resulted in very high levels of spontaneous apoptosis (FIG. 3A-3C), which were suppressed by DN-FADD overexpression (FIG. 4B).

c. Enhanced Apoptosis in SK-N-SH Cells Depleted of KIAA0358 is Due to Expression and Activation of Caspase-8.

In order to identify the mechanism of enhanced apoptosis induced by IG20-SV down-modulation, a question was whether specific caspases were activated in transduced SK-N-SH cells. Cells depleted of KIAA0358 (Mid, 13L and 13L+34E cells) showed enhanced expression of cleaved caspase-8. There was accompanying evidence for processing of caspase 3 (slightly reduced expression of pro-caspase-3), but no change in caspase-9 (FIG. 3D).

d. Manipulation of IG20-SVs in Other NB Cell Lines (SH-SY5Y and SK-N-BE(2)-C)?

Similarly, SH-SY5Y cells transduced with 13L and 13L+34E siRNAs showed enhanced apoptosis associated with prominent expression and activation of caspase-8 (FIG. 2). SK-N-BE(2)-C cells did not express KIAA0358 and IG20-SV4 so the siRNAs targeting these isoforms were not relevant in this cell line. Instead, IG20-SV4 and KIAA0358 were over-expressed in SK-N-BE(2)-C cells and the effect on caspase-8 activation were examined. Introduction of these isoforms had no effect on expression or activation of caspase-8 (FIG. 3) which was expressed at very low baseline levels in these cells.

### Example 4

### Treatment with TNF-α Enhances Apoptosis in NB Cells Expressing IG20-SV4 in a FADD-Dependent Manner, but does not Attenuate the Anti-Apoptotic Effect of KIAA0358

As a binding partner for the tumor necrosis factor receptor 1 (TNFRI), the IG20 gene promotes both pro-apoptotic and anti-apoptotic signals in Hela cells. Therefore, the apoptotic effect of TNF-α on SK-N-SH cells was tested. Treatment with TNFα enhanced apoptosis in cells transduced with shRNAs targeting the 13L exon and the combination of exons 13L and 34E (FIG. 4A). This induced sensitization to TNFα was significantly suppressed by DN-FADD over-expression (FIG. 4B). However, cells transduced with shRNA targeting exon 34 that did not alter endogenous expression of KIAA0358 and IG20-SV4, continued to be resistant to apoptosis even after TNF-α treatment (FIG. 4A).

### Example 5

### Over-Expression of KIAA0358 can Rescue SK-N-SH Cells from Spontaneous Apoptosis Induced by Down-Modulation of all IG20-SVs by Dampening Caspase-8 Activation

Silent mutations were created in cDNAs encoding KIAA0358 at sites corresponding to the 5^{th}, 7^{th}, 11^{th} and 14^{th} nucleotides of the Mid-shRNA target sequence. These mutations neither affected the amino-acid sequence nor protein expression. SK-N-SH cells stably expressing YFP-KIAA0358-Mut were generated. The Mid-shRNA was unable to down-modulate YFP-KIAA0358-Mut, but effectively down-modulated expression of all endogenous IG20-SVs (FIG. 5A). Expression of this KIAA0358 mutant was sufficient to rescue SK-N-SH cells from spontaneous apoptosis caused by Mid-shRNA transduction (FIG. 5B), confirming the anti-apoptotic properties of KIAA0358. These pro-survival effects were associated with nearly complete dampening of caspase-8 activation (FIG. 5C).

### Example 6

### Down-Modulation of KIAA0358 and Selective Expression of IG20-SV4 Modulates Expression of Caspase-8 in Caspase-8-Deficient SK-N-SH Cells

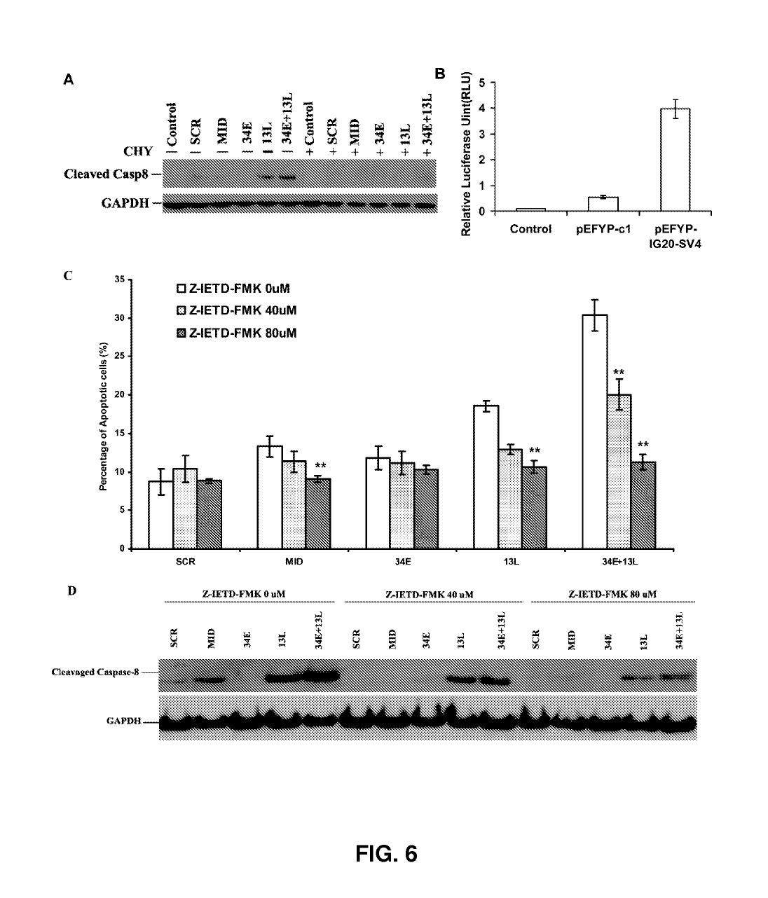

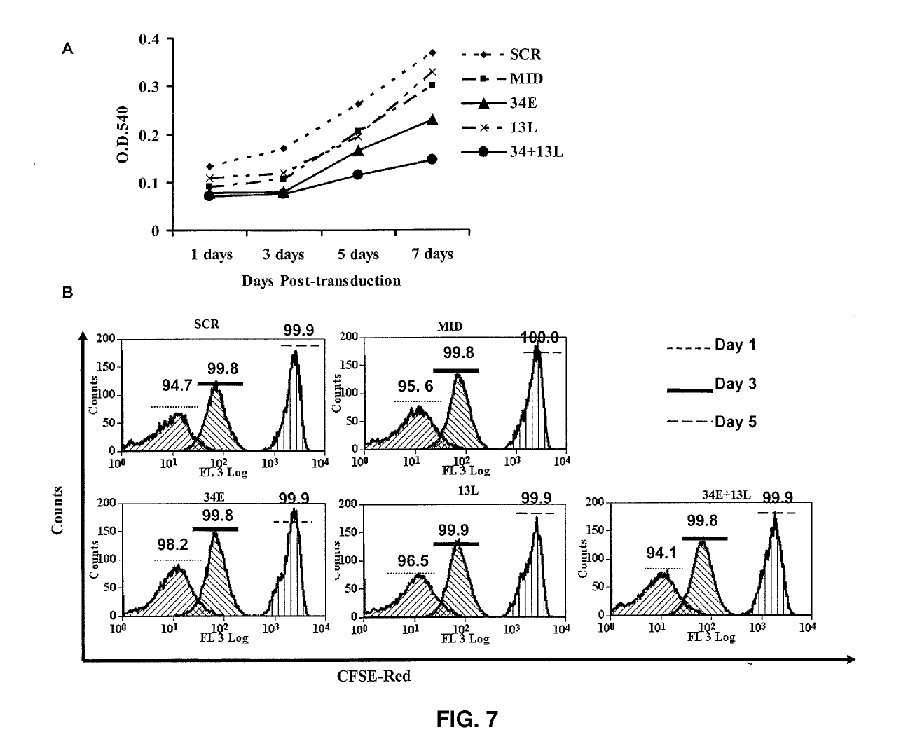

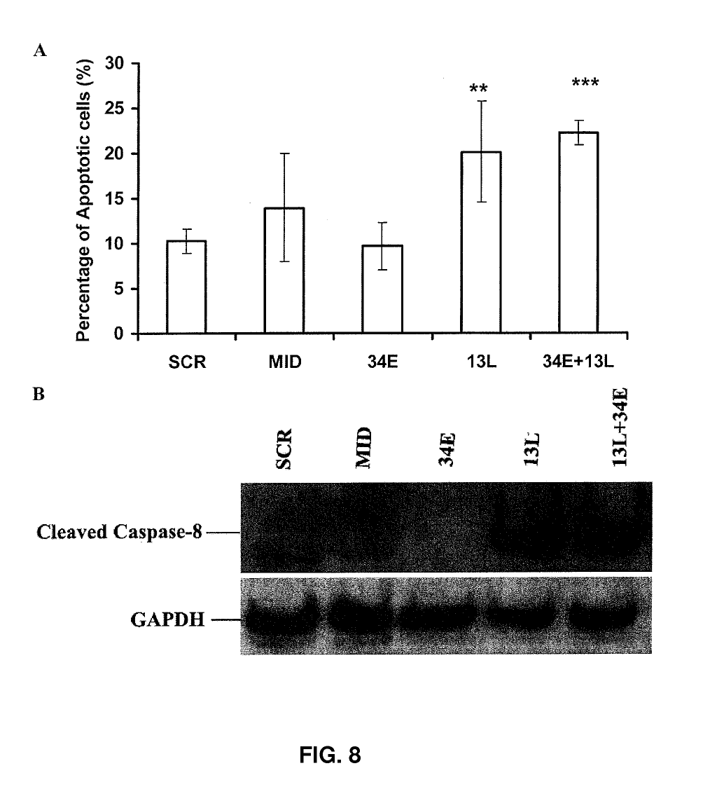

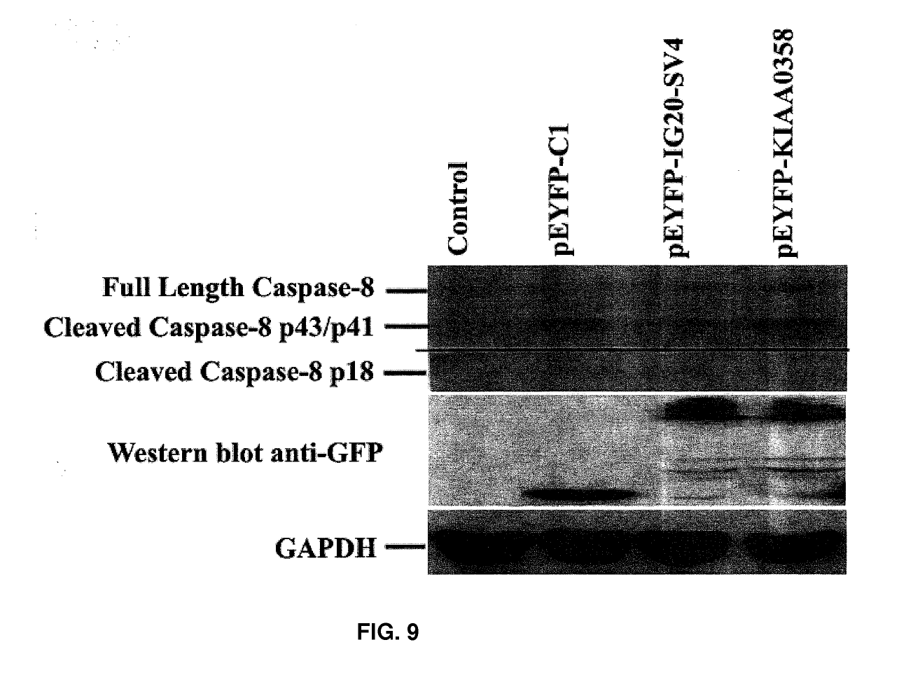

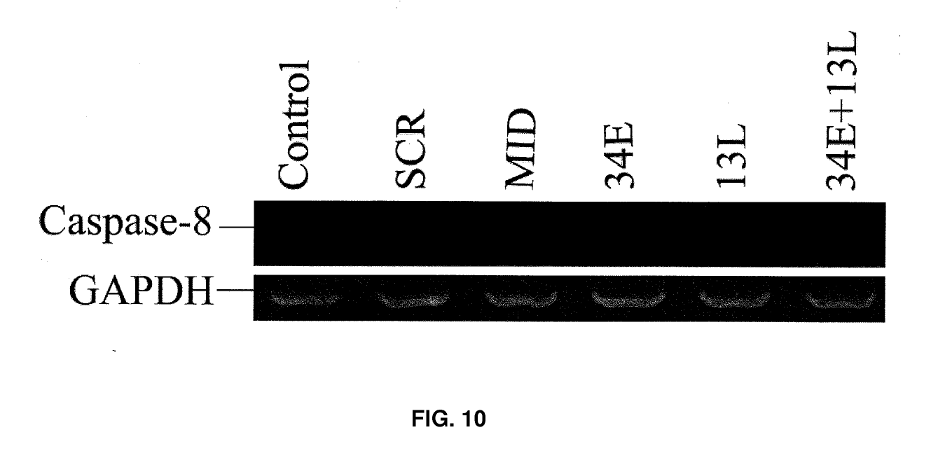

To determine whether the increased apoptosis induced utilizing 34+13L shRNA was due to modulation of the expression of caspase-8, the expression of caspase-8 transcripts was measured in SK-N-SH cells treated with the different combinations of siRNAs. SK-N-SH cells were found in which all isoforms were down-modulated leaving expression of IG20-SV4 unperturbed (13L+34E), expressed increased levels of caspase-8 mRNA compared to control cells (FIG. 4). To confirm that the increased expression of caspase-8 was due to induction of gene expression, the cells were exposed to 10 μg/mL cycloheximide as an inhibitor of new protein synthesis. This inhibited the expression of caspase-8 protein (FIG. 6A) suggesting that the effects of IG20-SV manipulation were mediated at the level of CASP8 gene expression. This result was further confirmed by using a luciferase assay, in which overexpression of IG20-SV4 caused a significant (4-fold) increase in activation of the CASP8 promoter compared to control or pEFYP-cl (empty vector) (FIG. 6B).

### Example 7

### Inhibition of Caspase-8 Effectively Decreases Apoptosis in 13L- and (34E+13L) Transduced SK-N-SH Cells in Dose Dependent Manner

The cells were pretreated with the specific caspase-8 inhibitor, Z-IETD-FMK (40 μM and 80 μM) which significantly attenuated the apoptotic effect caused by down-modulation of KIAA0358 in a dose-dependent fashion (FIG. 6C). The inhibitory effect of Z-IETD-FMK on caspase-8 expression was confirmed by western blot analysis (FIG. 6D). Inhibition of caspase 8 did not significantly affect apoptosis in cells treated with shRNA targeting 34E (FIG. 6C).

### Example 8

### Manipulating Expression of IG20-SV4 for Treatment of Neuroblastoma or a Related Disease Condition Including Other Cancers

A method to treat neuroblastoma or induce apoptosis in a neuroblastoma cell is to use siRNA that targets IG20 exon 34 and 13L to knock down or down regulate or silence all of the IG20 -SVs (splice variants) except IG20-SV4, which results in enhanced levels of IG20-SV4 expression. A suitable siRNA is either directly introduced into a neuroblastoma cell or expressed from a vector that generates shRNA and siRNA. This approach involves the expression of IG20-SV4 and relies on the cloning of shRNA that targets IG20 exon 34 and 13L into a suitable vector, e.g., a lentivirus vector, and transduction of 34E+13L sh-RNA into neuroblastoma cells causes knock down of all the IG20 -SVs except IG20-SV4.

A method to treat neuroblastoma or induce apoptosis in a neuroblastoma cell is to express IG20-SV4 in the cell. In an embodiment, the full-length coding sequence for the IG20-SV4 is used to overexpress the splice variant in a desired cell. In another embodiment, a fragment of the IG20-SV4 that is capable of inducing a desired response, e.g., induction of caspase 8 is preferred. For example, a cytotoxic portion of IG20-SV4 is identified and its corresponding DNA sequence is cloned it into an adenovirus expression vector and followed by introduction into the NB cells. Suitable domains of IG20-SV4 for use to induce apopotosis in a cancer cell include for example uDENN, DENN and dDENN domain in the N-terminal of IG20-SV4 (amino acid sequence 1-600aa), some DNA binding domains, like eukaryotic DNA topoisomeraes III DNA-binding domain, in the middle part (amino acid sequence 777-1300), and a domain in the RNA-binding Lupus La protein on the C-terminal end (amino acid sequence 1308-1368).

To evaluate the cytotoxic portion in IG20-SV4, constructs with truncated forms of IG20-SV4 expressing plasmids, which contain amino acid sequence 1308-1368, 777-1368, and 1-600 of IG20-SV4 are developed and tested for their ability to induce apoptosis (e.g., caspase-8 expression) by using a caspase-8 promoter luciferase system and western blot assay. The cytotoxic effects are also readily tested using a visual dye-based approach, e.g., by using trypan-blue and apoptosis assays in SK-N-SH cells.

### Example 9

### Identification of Small Molecules to Target Down Regulation of KIAA0358 or Upregulate IG20-SV4

Assays to identify small molecules or agents that specifically down regulate the expression of KIAA0358 in a neural cell for example a neuroblastoma cell are developed. For example, a library of compounds including small molecules, small peptides, peptide mimetics are screened for their ability to down regulate the expression of KIAA0358 or upregulate the expression of IG20-SV4 either at the mRNA level or at the protein level. In an embodiment, such a method includes for example monitoring the expression of KIAA0358 in response to a molecule of interest.

### Example 10

### Use of KIAA0358 to Ameliorate Neurodegenerative Diseases

Because down regulating the expression of KIAA0358 in a neural cell induces apoptosis, for example a neuroblastoma cell, overexpression of a coding sequence of KIAA0358 or a fragment thereof or providing KIAA0358 protein or a polypeptide thereof ameliorates cell death or rescue cell death in neurodegenerative disorders. For example, a neural specific promoter such as synapsin 1 is used to drive the expression of KIAA0358 in a neural cell. Synthetic peptides or polypeptides of KIAA0358 can also be used to reduce or minimize cell death associated with neurodegenerative diseases.

TABLE 1
Nucleotide sequence of Exon-specific siRNAs against IG20

| | | Targeting | |
|---|---|---|---|
| siRNA | Target Sequence | exon | Targeting isoform |
| SCR | 5′ TTTAACCGTTTACCGGCCT-3 | None | None |
| Mid | 5′ GTACCAGCTTCAGTCTTTC-3′ | Exon 15 | IG20pa, KIAA0358, MADD, [symbol] |
| | | | IG20-SV2, DENN-SV, IG20-SV4 |
| 34E | 5′ AGAGCTGAATCACATTAAA-3′ | Exon 34 | IG20pa, MADD, IG20- SV2, [symbol] |
| | | | DENN-SV |
| 13L | 5′CGGCGAATCTATGACAATC-3′ | Exon 13L | IG20pa, KIAA0358, MADD [symbol] |
| 34E + 13L | 5′ AGAGCTGAATCACATTAAA-3′ | Exon 34 | IG20pa, KIAA0358, MADD, [symbol] |
| | 5′CGGCGAATCTATGACAATC-3′ | Exon 13L | IG20-SV2, DENN-SV |

### Materials and Methods used in the Foregoing Examples

Cell culture: SK-N-SH, SH-SY5Y, and SK-N-BE(2)-C human neuroblastoma cell lines were purchased from ATCC and cultured according their instructions. Briefly, SK-N-SH cells were cultured in Dulbecco's modified Eagle's medium (Invitrogen, CA, USA) supplemented with 10% fetal bovine serum, 0.1 mM non-essential amino acids, 1.5 g/L sodium bicarbonate, 1.0 mM sodium pyruvate, and 100 units of penicillin/ml, and 100 μg of streptomycin/ml. SH-SY5Y and BE(2)-C cells were cultured in a 1:1 mixture of Eagle's minimum essential medium with non-essential amino acids and Ham's F12 medium (Invitrogen, CA, USA) supplemented with 10% fetal bovine serum and 100 units of penicillin and 100 lag of streptomycin/ml. The cell lines were maintained at 37° C. in a humidified chamber with 5% CO₂.

Design of small inhibitory RNAs. The siRNAs utilized herein are shown in FIG. 2A and FIG. 5. The siRNAs targeting exons 13L, 16E, and 15 (“Mid”) and the SCR (negative control) are disclosed. The siRNA targeting exon34 was designed using OligoEngine Workstation 2 and purchased from OligoEngine, Inc. (Seattle, Wash.). These siRNAs were screened in SK-N-SH cells and the most efficient were used to construct the 34E-shRNA lentivirus.

Plasmid construction. The siRNAs were cloned into the pSUPER vector using BgI II and HindTII sites to generate pSup-34 plasmids. The shRNA cassettes (including the H1 RNA promoter and the shRNA) were excised from pSup-34 using Xbal and Clal sites and ligated into the pNL-SIN-CMV-GFP vector to generate 34E lentivirus constructs. The pcTat, pcRev and pHIT/G were gifts from Dr. B. R. Cullen and Dr. T. J. Hope. The YFP-IG20pa plasmid was used as a backbone to subclone YFP-KIAA0358 from the corresponding pBKRSV plasmid using the BstZ 171 and BsiWI sites. The YFP-KIAA0358 and YFPIG20-SV4 mid-sh-RNA resistant mutant constructs were generated using the Quickchange XL site-directed mutagenesis kit (Stratagene, La Jolla, Calif., USA) according to the manufacturer's protocol. Briefly, the primers 5′-CGGAACCACAGTACAAGCTTTAGCCTCTCAAACCTCA CACTGCC-3′ (forward) and 5′-GGCAGTGTGAGGTTTGAGAGGCTAAAGCTTGTACTGTGGTT CCG-3′ (reverse) were used to insert four silent mutations (bold and underlined lettering) in the cDNAs without affecting the amino-acid sequence. Hind III restriction sites in the mutants, generated due to base substitutions, were used to identify positive clones that were further confirmed by sequencing. The caspase-8 promoter luciferase vector was constructed by PCR amplification of a 1.2 kb fragment from pBLCAT-Casp8 vector, and cloning into promega pGL4.17 luciferase vector at KpnI and XhoI site. The pBLCAT3 vector contain fragment −1161/+16 of caspase-8 promoter was gift from Dr. Silvano Ferrini's lab (DeAmbrosis et al., 2007).

Preparation of Lentivirus stocks. Lentivirus stocks were prepared as described by Lee et al., (2003), J. Virol.;77(22):11964-72. Briefly, subconfluent 293 FT cells grown in 100 mm plates were co-transfected with 10.8 mg of lentivirus vector, 0.6 mg pcRev, 0.6 mg of pcTat and 0.3 mg of pHIT/G using calcium phosphate. Culture medium was replaced after 16 h, and the supernatant was harvested at 40 h and filtered using a 0.45 mm filter. The optimal viral titer for each cell type was determined as the least amount of viral supernatant required to transduce at least 50% of target cells without apparent cytotoxicity.

RNA preparation. Total RNA extracted from human cerebral cortex, hippocampus, cerebellum, and human thyroid, skeletal muscle, lung and liver were purchased from BD Clontech (MountainView, Calif., USA). Total RNA extracted from primary NB was a gift from Dr. Jill Lahti's lab of St. Jude's Children's Research Hospital. For testing the efficiency of down-modulation of IG20 splice variants by different siRNAs, the transduced GFP positive SK-N-SH cells were sorted on the MoFlo™ High-Performance Cell Sorter (Dako Denmark, Glostrup, Denmark). Total RNA was extracted from 1×10⁶ GFP-positive NB cells and other described cell lines using Trizol reagent (Invitrogen Life Technologies, Carlsbad, Calif., USA).

Reverse transcription polymerase chain reaction. 1 μg of RNA was used for reverse transcription-polymerase chain reaction (RT-PCR) using the Super-Script III One-Step RT-PCR system (Invitrogen Life Technologies, Carlsbad, Calif., USA). Briefly, the cDNAs were synthesized at 50° C. for 30 minutes followed by incubation at 94° C. for 2 minutes. Subsequently, 30 cycles of PCR were carried out with denaturation at 94° C. for 50 seconds, annealing at 55° C. for 50 seconds and extension at 72° C. for variable time periods (as described herein); followed by a final incubation at 72° C. for 7 min. For amplifying exons 13L and 16, F-1 and B-1 primer pairs (5′-CGG GAC TCT GAC TCC GAA CCT AC-3′ and 5′-GCG GTT CAG CTT GCT CAG GAC-3′, respectively) were used, with 1 minute extension time. For amplifying exon 34, F4824 and B5092 primer pairs (5′ CTG CAG GTG ACC CTG GAA GGG ATC 3′ and 5′ TGT ACC CGG GTC AGC TAG AGA CAG GCC 3′, respectively) were used, with 30 second extension time. The sequence of GAPDH has been previously published (Ramaswamy et al., (2004), Oncogene; 23(36): 6083-6094). The PCR products were then separated on a 5% polyacrylamide gel.

Cell proliferation assay. Cell proliferation assays were performed according to the Vybrant MTT cell proliferation assay kit (V-13154, Molecular Probes, Invitrogen, CA, USA) instructions. Briefly, twenty-four-hour post-transduction, 1×10⁴ sorted GFP-positive SK-N-SH cells were plated onto 96-well plates. Every other day, cells were washed with PBS and labeled with 10 μL of 12 mM stock solution MTT in each well, incubated at 37° C. for 4 hours, washed with PBS. 50 μL, of DMSO was added to each well and mixed thoroughly with a pipette, and absorbance was recorded at 540 nm.

CFSE dilution assay. Twenty-four hours post-transduction, 1×105 SK-N-SH cells were stained with 2 mM SNARF-1 carboxylic acid, acetate, succinimidyl ester (S-22801, Molecular Probes, Invitrogen, CA, USA) for 15 minutes at 37° C. Cells were washed and either used immediately for FACS analysis, or plated into six-well plates. Every other day, cells were collected, washed and CFSE dilution, as an indicator of cell division, was determined in GFP-positive cells by FACS analysis at excitation/emission=480/640 nm.

DiIC staining. SK-N-SH (1.5×10⁵) cells were plated into six-well plates. Twenty-four hours later, cells were treated with different shRNA-expressing lentiviruses for 4 hours, washed and replenished with fresh warm medium immediately, and then every other day. At five days, the transduced cells were trypsinized with 0.05% trypsin, 0.53 mM EDTA and suspended in 1 mL warm PBS. Then, 5 μL of 10 μM DiIC (Molecular Probes, Invitrogen, Carlsbad, Calif.) was added and the cells were incubated at 37° C., 5% CO2 for 20 min. Cells were washed once by adding 2 mL of warm PBS, and resuspended in 500 μL of PBS. DiIC stained cells were analyzed on CyAn™ ADP Flow Cytometer (Dako Denmark, Glostrup, Denmark). Only GFP positive cells were gated and analyzed.

Apoptosis assay. Annexin V-phycoerythrin/7-amino-actinomycin D labeling was done according to the manufacturer's instructions (BD PharMingen) and samples were analyzed by flow cytometry. NB (1.5×10⁵) cells were plated into six-well plates. Twenty-four hours later, cells were treated with different shRNA-expressing lentiviruses for 4 h, washed and replenished with fresh warm medium immediately, and then every other day. At five days, the transduced cells were trypsinized and washed twice with cold PBS and then resuspended in 1× assay binding buffer. Annexin V-phycoerythrin/7-amino-actinomycin D labeling was performed at room temperature for 15 minutes before analysis by flow cytometry (BD FACScan). Only GFP positive cells were gated and analyzed.

Caspase-8 inhibition. At 3 days post-transduction with different shRNAs, SK-N-SH cells were treated with 40 μM and 80 μM of Z-IETD-FMK (BD PharMingen) for an additional two days, or with 10 μg/ml cycloheximide (Sigma) for an additional day. Collected cells were either subjected to Annexin V-PE/7-AAD staining followed by FACS or western blot analysis to determine active caspases.

Western Blot Analysis. Different shRNA-expressing, lentivirus-transduced NB cells were trypsinized and washed with phosphate-buffered saline and lysed at 0° C. for 30 min in a lysis buffer (20 mM Hepes, pH 7.4, 2 mM EGTA, 420 mM NaCL, 1% Triton X-100, 10% glycerol, 1 mM dithiothreitol, 1 mM phenylmethylsulfonyl fluoride, 10 μg/ml leupeptin, 10 μ/ml aprotinin, 1 mM Na₃VO₄, and 5 mM NaF). The protein content was determined using a dye-binding microassay (Bio-Rad), and after boiling the samples for 2 min in a 1× SDS protein sample buffer, 20 μg of protein per lane was loaded and separated on 10% SDS-polyacrylamide gel. The proteins were blotted onto Hybond ECL membranes (Amersham Biosciences). After electroblotting, the membranes were blocked with Tris-buffered saline with Tween-20 (TBST 10 mM Tris-HCI, pH 7.4, 150 mM NaCl, 0.1% Tween-20) containing 5% milk, and were incubated with antibodies diluted in a 5% BSA TBST buffer that can detect cleaved caspase-8 (Santa Cruz, C-20), caspase-9 (Cell signaling), and full length caspase-3 (R & D system, 84803) overnight. The primary antibody dilutions were those recommended by the manufacturer. The membranes were then washed, incubated with the appropriate secondary antibodies (1:5,000) in a blocking buffer for 1 h, and repeatedly washed. Proteins were detected using an enhanced chemiluminescence plus western blotting detection system (Amersham, UK). The anti-GAPDH-HRP (abcam) antibodies were used as loading controls.

Transient transfections and luciferase assays. 1.5×10⁵ SK-N-SH cells were seeded per well in 12-well plates and cotransfected them with either 1.6 μg of pEYFP-C1 or pEYFP-IG20-SV4, 1 μg of pGL4.17 (a promoterless control) or 1 μg of pGL4.17-caspase-8 promoter. 20 ng of pSV40-Renilla luciferase vector was cotransfected as a normalizing control. Transfections were carried out in triplicate. After 48 h of incubation, cells were collected and analyzed for luciferase activity with the Dual-Luciferase Reporter Assay System (Promega).

Dominant-negative FADD (pcDNA-DN-FADD) or control vector (pcDNA3.1) were transfected (5 μg each) into 6×10⁶ SK-N-SH cells, and distributed into 6-well plate. To increase the transfection efficiency of DN-FADD, nucleofection® from Amaxa biosystems was used. After 24 hours culture, the cells were either transduced with SCR or 34+13L sh-RNA. At 3 days post-transduction, the cells were treated or un-treated with 10 ng/ml TNFα for 48 hrs. The cells were trypsinized and stained with Annexin V-PE/7-AAD for FACS analysis. Only GFP positive cells were gated and analyzed.

Statistical analysis. All results are expressed as mean±SE. Student's t test was used to determine P values using Microsoft Excel Software (version 2003).

SEQUENCES
KIAA0358 nucleic acid sequence
(GenBank Acc. No. AB002356)
ACTCAGATCTTCCATGGTGCAAAAGAAGAAGTTCTGTCCTCGGTTACTTG
ACTATCTAGTGATCGTAGGGGCCAGGCACCCGAGCAGTGATAGCGTGGCC
CAGACTCCTGAATTGCTACGGCGATACCCCTTGGAGGATCACACTGAGTT
TCCCCTGCCCCCAGATGTAGTGTTCTTCTGCCAGCCCGAGGGCTGCCTGA
GCGTGCGGCAGCGGCGCATGAGCCTTCGGGATGATACCTCTTTTGTCTTC
ACCCTCACTGACAAGGACACTGGAGTCACGCGATATGGCATCTGTGTTAA
CTTCTACCGCTCCTTCCAAAAGCGAATCTCTAAGGAGAAGGGGGAAGGTG
GGGCAGGGTCCCGTGGGAAGGAAGGAACCCATGCCACCTGTGCCTCAGAA
GAGGGTGGCACTGAGAGCTCAGAGAGTGGCTCATCCCTGCAGCCTCTCAG
TGCTGACTCTACCCCTGATGTGAACCAGTCTCCTCGGGGCAAACGCCGGG
CCAAGGCGGGGAGCCGCTCCCGCAACAGTACTCTCACGTCCCTGTGCGTG
CTCAGCCACTACCCTTTCTTCTCCACCTTCCGAGAGTGTTTGTATACTCT
CAAGCGCCTGGTGGACTGCTGTAGTGAGCGCCTTCTGGGCAAGAAACTGG
GCATCCCTCGAGGCGTACAAAGGGACACCATGTGGCGGATCTTTACTGGA
TCGCTGCTGGTAGAGGAGAAGTCAAGTGCCCTTCTGCATGACCTTCGAGA
GATTGAGGCCTGGATCTATCGATTGCTGCGCTCCCCAGTACCCGTCTCTG
GGCAGAAGCGAGTAGACATCGAGGTCCTACCCCAAGAGCTCCAGCCAGCT
CTGACCTTTGCTCTTCCAGACCCATCTCGATTCACCCTAGTGGATTTCCC
ACTGCACCTTCCCTTGGAACTTCTAGGTGTGGACGCCTGTCTCCAGGTGC
TAACCTGCATTCTGTTAGAGCACAAGGTGGTGCTACAGTCCCGAGACTAC
AATGCACTCTCCATGTCTGTGATGGCATTCGTGGCAATGATCTACCCACT
GGAATATATGTTTCCTGTCATCCCGCTGCTACCCACCTGCATGGCATCAG
CAGAGCAGCTGCTGTTGGCTCCAACCCCGTACATCATTGGGGTTCCTGCC
AGCTTCTTCCTCTACAAACTGGACTTCAAAATGCCTGATGATGTATGGCT
AGTGGATCTGGACAGCAATAGGGTGATTGCCCCCACCAATGCAGAAGTGC
TGCCTATCCTGCCAGAACCAGAATCACTAGAGCTGAAAAAGCATTTAAAG
CAGGCCTTGGCCAGCATGAGTCTCAACACCCAGCCCATCCTCAATCTGGA
GAAATTTCATGAGGGCCAGGAGATCCCCCTTCTCTTGGGAAGGCCTTCTA
ATGACCTGCAGTCCACACCGTCCACTGAATTCAACCCACTCATCTATGGC
AACGATGTGGATTCTGTGGATGTTGCAACCAGGGTTGCCATGGTACGGTT
CTTCAATTCCGCCAACGTGCTGCAGGGATTTCAGATGCACACGCGTACCC
TGCGCCTCTTTCCTCGGCCTGTGGTAGCTTTTCAAGCTGGCTCCTTTCTA
GCCTCACGTCCCCGGCAGACTCCTTTTGCCGAGAAATTGGCCAGGACTCA
GGCTGTGGAGTACTTTGGGGAATGGATCCTTAACCCCACCAACTATGCCT
TTCAGCGAATTCACAACAATATGTTTGATCCAGCCCTGATTGGTGACAAG
CCAAAGTGGTATGCTCATCAGCTGCAGCCTATCCACTATCGCGTCTATGA
CAGCAATTCCCAGCTGGCTGAGGCCCTGAGTGTACCACCAGAGCGGGACT
CTGACTCCGAACCTACTGATGATAGTGGCAGTGATAGTATGGATTATGAC
GATTCAAGCTCTTCTTACTCCTCCCTTGGTGACTTTGTCAGTGAAATGAT
GAAATGTGACATTAATGGTGATACTCCCAATGTGGACCCTCTGACACATG
CAGCACTGGGGGATGCCAGCGAGGTGGAGATTGACGAGCTGCAGAATCAG
AAGGAAGCAGAAGAGCCTGGCCCAGACAGTGAGAACTCTCAGGAAAACCC
CCCACTGCGCTCCAGCTCTAGCACCACAGCCAGCAGCAGCCCCAGCACTG
TCATCCACGGAGCCAACTCTGAACCTGCTGACTCTACGGAGATGGATGAT
AAGGCAGCAGTAGGCGTCTCCAAGCCCCTCCCTTCCGTGCCTCCCAGCAT
TGGCAAATCGAACGTGGACAGACGTCAGGCAGAAATTGGAGAGGGGTCAG
TGCGCCGGCGAATCTATGACAATCCATACTTCGAGCCCCAATATGGCTTT
CCCCCTGAGGAAGATGAGGATGAGCAGGGGGAAAGTTACACTCCCCGATT
CAGCCAACATGTCAGTGGCAATCGGGCTCAAAAGCTGCTGCGGCCCAACA
GCTTGAGACTGGCAAGTGACTCAGATGCAGAGTCAGACTCTCGGGCAAGC
TCTCCCAACTCCACCGTCTCCAACACCAGCACCGAGGGCTTCGGGGGCAT
CATGTCTTTTGCCAGCAGCCTCTATCGGAACCACAGTACAAGCTTTAGCC
TCTCAAACCTCACACTGCCCACCAAAGGTGCCCGAGAGAAGGCCACGCCC
TTCCCCAGTCTGAAAGTATTTGGGCTAAATACTCTAATGGAGATTGTTAC
TGAAGCCGGCCCCGGGAGTGGTGAAGGAAACAGGAGGGCGTTAGTGGATC
AGAAGTCATCTGTCATTAAACACAGCCCAACAGTGAAAAGAGAACCTCCA
TCACCCCAGGGTCGATCCAGCAATTCTAGTGAGAACCAGCAGTTCCTGAA
GGAGGTGGTGCACAGCGTGCTGGACGGCCAGGGAGTTGGCTGGCTCAACA
TGAAAAAGGTGCGCCGGCTGCTGGAGAGCGAGCAGCTGCGAGTCTTTGTC
CTGAGCAAGCTGAACCGCATGGTGCAGTCAGAGGACGATGCCCGGCAGGA
CATCATCCCGGATGTGGAGATCAGTCGGAAGGTGTACAAGGGAATGTTAG
ACCTCCTCAAGTGTACAGTCCTCAGCTTGGAGCAGTCCTATGCCCACGCG
GGTCTGGGTGGCATGGCCAGCATCTTTGGGCTTTTGGAGATTGCCCAGAC
CCACTACTATAGTAAAGAACCAGACAAGCGGAAGAGAAGTCCAACAGAAA
GTGTAAATACCCCAGTTGGCAAGGATCCTGGCCTAGCTGGGCGGGGGGAC
CCAAAGGCTATGGCACAACTGAGAGTTCCACAACTGGGACCTCGGGCACC
AAGTGCCACAGGAAAGGGTCCTAAGGAACTGGACACCAGAAGTTTAAAGG
AAGAAAATTTTATAGCATCTATTGAATTGTGGAACAAGCACCAGGAAGTG
AAAAAGCAAAAAGCTTTGGAAAAACAGAGGCCTGAAGTAATCAAACCTGT
CTTTGACCTTGGTGAGACAGAGGAGAAAAAGTCCCAGATCAGCGCAGACA
GTGGTGTGAGCCTGACGTCTAGTTCCCAGAGGACTGATCAAGACTCTGTC
ATCGGCGTGAGTCCAGCTGTTATGATCCGCAGCTCAAGTCAGGATTCTGA
AGTTAGCACCGTGGTGAGTAATAGCTCTGGAGAGACCCTTGGAGCTGACA
GTGACTTGAGCAGCAATGCAGGTGATGGACCAGGTGGCGAGGGCAGTGTT
CACCTGGCAAGCTCTCGGGGCACTTTGTCTGATAGTGAAATTGAGACCAA
CTCTGCCACAAGCACCATCTTTGGTAAAGCCCACAGCTTGAAGCCAAGCA
TAAAGGAGAAGCTGGCAGGCAGCCCCATTCGTACTTCTGAAGATGTGAGC
CAGCGAGTCTATCTCTATGAGGGACTCCTAGGAAGGGACAAAGGATCCAT
GTGGGACCAGTTAGAGGATGCAGCTATGGAGACCTTTTCTATAAGCAAAG
AGCGTTCTACTTTATGGGACCAAATGCAATTCTGGGAAGATGCCTTCTTA
GATGCTGTGATGTTGGAGAGAGAAGGGATGGGTATGGACCAGGGTCCCCA
GGAAATGATCGACAGGTACCTGTCCCTTGGAGAACATGACCGGAAGCGCC
TGGAAGATGATGAAGATCGCTTGCTGGCCACACTTCTGCACAACCTCATC
TCCTACATGCTGCTGATGAAGGTAAATAAGAATGACATCCGCAAGAAGGT
GAGGCGCCTAATGGGAAAGTCGCACATTGGGCTTGTGTACAGCCAGCAAA
TCAATGAGGTGCTTGATCAGCTGGCGAACCTGAATGGACGCGATCTCTCT
ATCTGGTCCAGTGGCAGCCGGCACATGAAGAAGCAGACATTTGTGGTACA
TGCAGGGACAGATACAAACGGAGATATCTTTTTCATGGAGGTGTGCGATG
ACTGTGTGGTGTTGCGTAGTAACATCGGAACAGTGTATGAGCGCTGGTGG
TACGAGAAGCTCATCAACATGACCTACTGTCCCAAGACGAAGGTGTTGTG
CTTGTGGCGTAGAAATGGCTCTGAGACCCAGCTCAACAAGTTCTATACTA
AAAAGTGTCGGGAGCTGTACTACTGTGTGAAGGACAGCATGGAGCGCGCT
GCCGCCCGACAGCAAAGCATCAAACCCGGACCTGAATTGGGTGGCGAGTT
CCCTGTGCAGGACCTGAAGACTGGTGAGGGTGGCCTGCTGCAGGTGACCC
TGGAAGGGATCAACCTCAAATTCATGCACAATCAGTTCCTGAAATTAAAG
AAGTGGTGAGCCACAAGTACAAGACACCAATGGCCCACGAAATCTGCTAC
TCCGTATTATGTCTCTTCTCGTACGTGGCTGCAGTTCATAGCAGTGAGGA
AGATCTCAGAACCCCGCCC
KIAA0358 Amino Acid Sequence
(GenBank Acc. No. BAA20814.2)
LESEQLRVFVLSKLNRMVQSEDDARQDIIPDVEISRKVYKGMLDLLKCTV
LSLEQSYAHAGLGGMASIFGLLEIAQTHYYSKEPDKRKRSPTESVNTPVG
KDPGLAGRGDPKAMAQLRVPQLGPRAPSATGKGPKELDTRSLKEENFIAS
IELWNKHQEVKKQKALEKQRPEVIKPVFDLGETEEKKSQISADSGVSLTS
SSQRTDQDSVIGVSPAVMIRSSSQDSEVSTVVSNSSGETLGADSDLSSNA
GDGPGGEGSVHLASSRGTLSDSEIETNSATSTIFGKAHSLKPSIKEKLAG
SPIRTSEDVSQRVYLYEGLLGRDKGSMWDQLEDAAMETFSISKERSTLWD
QMQFWEDAFLDAVMLEREGMGMDQGPQEMIDRYLSLGEHDRKRLEDDEDR
LLATLLHNLISYMLLMKVNKNDIRKKVRRLMGKSHIGLVYSQQINEVLDQ
LANLNGRDLSIWSSGSRHMKKQTFVVHAGTDTNGDIFFMEVCDDCVVLRS
NIGTVYERWWYEKLINMTYCPKTKVLCLWRRNGSETQLNKFYTKKCRELY
YCVKDSMERAAARQQSIKPGPELGGEFPVQDLKTGEGGLLQVTLEGINLK
FMHNQFLKLKKW
IG20-SV4 nucleic acid sequence
(GenBank Acc. No. AF440434)
CCCGCTGCCCAGGATTGGTAGACTCCACCGCTCGGCAGCCGGCTTCCCTG
CTCGGACGCCGAGCACCGCCAAAGCGCACTTCGATTTTCAGAATTCCTCC
TGGGAATGCTGACTCCTTGCTTGGTGCCCTGATGCTTCTCTGAGATAAAC
TGATGAATTGGAACCATGGTGCAAAAGAAGAAGTTCTGTCCTCGGTTACT
TGACTATCTAGTGATCGTAGGGGCCAGGCACCCGAGCAGTGATAGCGTGG
CCCAGACTCCTGAATTGCTACGGCGATACCCCTTGGAGGATCACACTGAG
TTTCCCCTGCCCCCAGATGTAGTGTTCTTCTGCCAGCCCGAGGGCTGCCT
GAGCGTGCGGCAGCGGCGCATGAGCCTTCGGGATGATACCTCTTTTGTCT
TCACCCTCACTGACAAGGACACTGGAGTCACGCGATATGGCATCTGTGTT
AACTTCTACCGCTCCTTCCAAAAGCGAATCTCTAAGGGGAAGGGGGAAGG
TGGGGCAGGGTCCCGTGGGAAGGAAGGAACCCATGCCACCTGTGCCTCAG
AAGAGGGTGGCACTGAGAGCTCAGAGAGTGGCTCATCCCTGCAGCCTTTC
AGTGCTGACTCTACCCCTGATGTGAACCAGTCTCCTCGGGGCAAACGCCG
GGCCAAGGCGGGGAGCCGCTCCCGCAACAGTACTCTCACGTCCCTGTGCG
TGCTCAGCCACTACCCTTTCTTCTCCACCTTCCGAGAGTGTTTGTATACT
CTCAAGCGCCTGGTGGACTGCTGTAGTGAGCGCCTTCTGGGCAAGAAACT
GGGCATCCCTCGAGGCGTACAAAGGGACACCATGTGGCGGATCTTTACTG
GATCGCTGCTGGTAGAGGAGAAGTCAAGTGCCCTTCTGCATGACCTTCGA
GAGATTGAGGCCTGGATCTATCGATTGCTGCGCTCCCCAGTACCCGTCTC
TGGGCAGAAGCGAGTAGACATCGAGGTCCTACCCCAAGAGCTCCAGCCAG
CTCTGACCTTTGCTCTTCCAGACCCATCTCGATTCACCCTAGTGGATTTC
CCACTGCACCTTCCCTTGGAACTTCTAGGTGTGGACGCCTGTCTCCAGTT
GCTAACCTGCATTCTGTTAGAGCACAAGGTGGTGCTACAGTCCCGAGACT
ACAATGCACTCTCCATGTCTGTGATGGCATTCGTGGCAATGATCTACCCA
CTGGAGTATATGTTTCCTGTCATCCCGCTGCTACCCACCTGCATGGCATC
AGCAGAGCAGCTGCTGTTGGCTCCAACCCCGTACATCATTGGGGTTCCTG
CCAGCTTCTTCCTCTACAAACTGGACTTCAAAATGCCTGATGATGTATGG
CTAGTGGATCTGGACAGCAATAGGGTGATTGCCCCCACCAATGCAGAAGT
GCTGCCTATCCTGCCAGAACCAGAATCACTAGAGCTGAAAAAGCATTTAA
AGCAGGCCTTGGCCAGCATGAGTCTCAACACCCAGCCCATCCTCAATCTG
GAGAAATTTCATGAGGGCCAGGAGATCCCCCTTCTCTTGGGAAGGCCTTC
TAATGACCTGCAGTCCACACCGTCCACTGAATTCAACCCACTCATCTATG
GCAATGATGCGGATTCTGTGGATGTTGCAACCAGGGTTGCCATGGTACGG
TTCTTCAATTCCGCCAACGTGCTGCAGGGATTTCAGATGCACACGCGTAC
CCTGCGCCTCTTTCCTCGGCCTGTGGTAGCTTTTCAAGCTGGCTCCTTTC
TAGCCTCACGTCCCCGGCAGACTCCTTTTGCCGAGAAATTGGCCAGGACT
CAGGCTGTGGAGTACTTTGGGGAATGGATCCTTAACCCCACCAACTATGC
CTTTCAGCGAATTCACAACAATATGTTTGATCCAGCCCTGATTGGTGACA
AGCCAAAGTGGTATGCTCATCAGCTGCAGCCTATCCACTATCGCGTCTAT
GACAGCAATTCCCAGCTGGCTGAGGCCCTGAGTGTACCACCAGAGCGGGA
CTCTGACTCCGAACCTACTGATGATAGTGGCAGTGATAGTATGGATTATG
ACGATTCAAGCTCTTCTTACTCCTCCCTTGGTGACTTTGTCAGTGAAATG
ATGAAATGTGACATTAATGGTGATACTCCCAATGTGGACCCTCTGACACA
TGCAGCACTGGGGGATGCCAGCGAGGTGGAGATTGACGAGCTGCAGAATC
AGAAGGAAGCAGAAGAGCCTGGCCCAGACAGTGAGAACTCTCAGGAAAAC
CCCCCACTGCGCTCCAGCTCTAGCACCACAGCCAGCAGCAGCCCCAGCAC
TGTCATCCACGGAGCCAACTCTGAACCTGCTGACTCTACGGAGATGGATG
ATAAGGCAGCAGTAGGCGTCTCCAAGCCCCTCCCTTCCGTGCCTCCCAGC
ATTGGCAAATCGAACGTGGACAGACGTCAGGCAGAAATTGGAGAGGGGGC
TCAAAAGCTGCTGCGGCCCAACAGCTTGAGACTGGCAAGTGACTCAGATG
CAGAGTCAGACTCTCGGGCAAGCTCTCCCAACTCCACCGTCTCCAACACC
AGCACCGAGGGCTTCGGGGGCATCATGTCTTTTGCCAGCAGCCTCTATCG
GAACCACAGTACCAGCTTCAGTCTTTCAAACCTCACACTGCCCACCAAAG
GTGCCCGAGAGAAGGCCACGCCCTTCCCCAGTCTGAAAGGAAACAGGAGG
GCGTTAGTGGATCAGAAGTCATCTGTCATTAAACACAGCCCAACAGTGAA
AAGAGAACCTCCATCACCCCAGGGTCGATCCAGCAATTCTAGTGAGAACC
AGCAGTTCCTGAAGGAGGTGGTGCACAGCGTGCTGGACGGCCAGGGAGTT
GGCTGGCTCAACATGAAAAAGGTGCGCCGGCTGCTGGAGAGCGAGCAGCT
GCGAGTCTTTGTCCTGAGCAAGCTGAACCGCATGGTGCAGTCAGAGGACG
ATGCCCGGCAGGACATCATCCCGGATGTGGAGATCAGTCGGAAGGTGTAC
AAGGGAATGTTAGACCTCCTCAAGTGTACAGTCCTCAGCTTGGAGCAGTC
CTATGCCCACGCGGGTCTGGGTGGCATGGCCAGCATCTTTGGGCTTTTGG
AGATTGCCCAGACCCACTACTATAGTAAAGAACCAGACAAGCGGAAGAGA
AGTCCAACAGAAAGTGTAAATACCCCAGTTGGCAAGGATCCTGGCCTAGC
TGGGCGGGGGGACCCAAAGGCTATGGCACAACTGAGAGTTCCACAACTGG
GACCTCGGGCACCAAGTGCCACAGGAAAGGGTCCTAAGGAACTGGACACC
AGAAGTTTAAAGGAAGAAAATTTTATAGCATCTATTGGGCCTGAAGTAAT
CAAACCTGTCTTTGACCTTGGTGAGACAGAGGAGAAAAAGTCCCAGATCA
GCGCAGACAGTGGTGTGAGCCTGACGTCTAGTTCCCAGAGGACTGATCAA
GACTCTGTCATCGGCGTGAGTCCAGCTGTTATGATCCGCAGCTCAAGTCA
GGATTCTGAAGTTAGCACCGTGGTGAGTAATAGCTCTGGAGAGACCCTTG
GAGCTGACAGTGACTTGAGCAGCAATGCAGGTGATGGACCAGGTGGCGAG
GGCAGTGTTCACCTGGCAAGCTCTCGGGGCACTTTGTCTGATAGTGAAAT
TGAGACCAACTCTGCCACAAGCACCATCTTTGGTAAAGCCCACAGCTTGA
AGCCATGCATAAAGGAGAAGCTGGCAGGCAGCCCCATTCGTACTTCTGAA
GATGTGAGCCAGCGAGTCTATCTCTATGAGGGACTCCTAGGCAAAGAGCG
TTCTACTTTATGGGACCAAATGCAATTCTGGGAAGATGCCTTCTTAGATG
CTGTGATGTTGGAGAGAGAAGGGATGGGTATGGACCAGGGTCCCCAGGAA
ATGATCGACAGGTACCTGTCCCTTGGAGAACATGACCGGAAGCGCCTGGA
AGATGATGAAGATCGCTTGCTGGCCACACTTCTGCACAACCTCATCTCCT
ACATGCTGCTGATGAAGGTAAATAAGAATGACATCCGCAAGAAGGTGAGG
CGCCTAATGGGAAAGTCGCACATTGGGCTTGTGTACAGCCAGCAAATCAA
TGAGGTGCTTGATCAGCTGGCGAACCTGAATGGACGCGATCTCTCTATCT
GGTCCAGTGGCAGCCGGCACATGAAGAAGCAGACATTTGTGGTACATGCA
GGGACAGATACAAACGGAGATATCTTTTTCATGGAGGTGTGCGATGACTG
TGTGGTGTTGCGTAGTAACATCGGAACAGTGTATGAGCGCTGGTGGTACG
AGAAGCTCATCAACATGACCTACTGTCCCAAGACGAAGGTGTTGTGCTTG
TGGCGTAGAAATGGCTCTGAGACCCAGCTCAACAAGTTCTATACTAAAAA
GTGTCGGGAGCTGTACTACTGTGTGAAGGACAGCATGGAGCGCGCTGCCG
CCCGACAGCAAAGCATCAAACCCGGACCTGAATTGGGTGGCGAGTTCCCT
GTGCAGGACCTGAAGACTGGTGAGGGTGGCCTGCTGCAGGTGACCCTGGA
AGGGATCAACCTCAAATTCATGCACAATCAGTTCCTGAAATTAAAGAAGT
GGTGAGCCACAAGTACAAGACACCAATGGCCCACGAAATCTGCTACTCCG
TATTATGTCTCTTCTCGTACGTGGCTGCAGTTCATAGCAGTGAGGAAGAT
CTCAGAACCCCGCCCCGGCCTGTCTCTAGCTGATGGAGAGGGGCTACGCA
GCTGCCCCAGCCCAGGGCACGCCCCTGGCCCCTTGCTGTTCCCAAGTGCA
CGATGCTGCTGTGACTGAGGAGTGGATGATGCTCGTGTGTCCTCTGCAAC
CCCCCTGCTGTGGCTTGGTTGGTTACCGGTTATGTGTCCCTCTGAGTGTG
TCTTGAGCGTGTCCACCTTCTCCCTCTCCACTCCCAGAAGACCAAACTGC
CTTCCCCTCAGGGCTCAAGAATGTGTACAGTCTGTGGGGCCGGTGTGAAC
CCACTATTTTGTGTCCTTGAGACATTTGTGTTGTGGTTCCTTGTCCTTGT
CCCTGGCGTTATAACTGTCCACTGCAAGAGTCTGGCTCTCCCTTCTCTGT
GACCCGGCATGACTGGGCGCCTGGAGCAGTTCACTCTGTGAGGAGTGAGG
GAACCCTGGGGCTCACCCTCTCAGAGGAAGGGCACAGAGAGGAAGGGAAG
AATTGGGGGGCAGCCGGAGTGAGTGGCAGCCTCCCTGCTTCCTTCTGCAT
TCCCAAGCCGGCAGCCACTGCCCAGGGCCCGCAGTGTTGGCTGCTGCCTG
CCACAGCCTCTGTGACTGCAGTGGAGCGGCGAATTCCCTGTGGCCTGCCA
CGCCTTCGGCATCAGAGGATGGAGTGGTCGAGGCTAGTGGAGTCCCAGGG
ACCGCTGGCTGCTCTGCCTGAGCATCAGGGAGGGGGCAGGAAAGACCAAG
CTGGGTTTGCACATCTGTCTGCAGGCTGTCTCTCCAGGCACGGGGTGTCA
GGAGGGAGAGACAGCCTGGGTATGGGCAAGAAATGACTGTAAATATTTCA
GCCCCACATTATTTATAGAAAATGTACAGTTGTGTGAATGTGAAATAAAT
GTCCTCAATTCCCAAAAAA
IG20-SV4 amino acid sequence
(GenBank Acc. No. AAL35261.1)
MVQKKKFCPRLLDYLVIVGARHPSSDSVAQTPELLRRYPLEDHT
EFPLPPDVVFFCQPEGCLSVRQRRMSLRDDTSFVFTLTDKDTGVTRYGIC
VNFYRSFQKRISKGKGEGGAGSRGKEGTHATCASEEGGTESSESGSSLQP
FSADSTPDVNQSPRGKRRAKAGSRSRNSTLTSLCVLSHYPFFSTFRECLY
TLKRLVDCCSERLLGKKLGIPRGVQRDTMWRIFTGSLLVEEKSSALLHDL
REIEAWIYRLLRSPVPVSGQKRVDIEVLPQELQPALTFALPDPSRFTLVD
FPLHLPLELLGVDACLQLLTCILLEHKVVLQSRDYNALSMSVMAFVAMIY
PLEYMFPVIPLLPTCMASAEQLLLAPTPYIIGVPASFFLYKLDFKMPDDV
WLVDLDSNRVIAPTNAEVLPILPEPESLELKKHLKQALASMSLNTQPILN
LEKFHEGQEIPLLLGRPSNDLQSTPSTEFNPLIYGNDADSVDVATRVAMV
RFFNSANVLQGFQMHTRTLRLFPRPVVAFQAGSFLASRPRQTPFAEKLAR
TQAVEYFGEWILNPTNYAFQRIHNNMFDPALIGDKPKWYAHQLQPIHYRV
YDSNSQLAEALSVPPERDSDSEPTDDSGSDSMDYDDSSSSYSSLGDFVSE
MMKCDINGDTPNVDPLTHAALGDASEVEIDELQNQKEAEEPGPDSENSQE
NPPLRSSSSTTASSSPSTVIHGANSEPADSTEMDDKAAVGVSKPLPSVPP
SIGKSNVDRRQAEIGEGAQKLLRPNSLRLASDSDAESDSRASSPNSTVSN
TSTEGFGGIMSFASSLYRNHSTSFSLSNLTLPTKGAREKATPFPSLKGNR
RALVDQKSSVIKHSPTVKREPPSPQGRSSNSSENQQFLKEVVHSVLDGQG
VGWLNMKKVRRLLESEQLRVFVLSKLNRMVQSEDDARQDIIPDVEISRKV
YKGMLDLLKCTVLSLEQSYAHAGLGGMASIFGLLEIAQTHYYSKEPDKRK
RSPTESVNTPVGKDPGLAGRGDPKAMAQLRVPQLGPRAPSATGKGPKELD
TRSLKEENFIASIGPEVIKPVFDLGETEEKKSQISADSGVSLTSSSQRTD
QDSVIGVSPAVMIRSSSQDSEVSTVVSNSSGETLGADSDLSSNAGDGPGG
EGSVHLASSRGTLSDSEIETNSATSTIFGKAHSLKPCIKEKLAGSPIRTS
EDVSQRVYLYEGLLGKERSTLWDQMQFWEDAFLDAVMLEREGMGMDQGPQ
EMIDRYLSLGEHDRKRLEDDEDRLLATLLHNLISYMLLMKVNKNDIRKKV
RRLMGKSHIGLVYSQQINEVLDQLANLNGRDLSIWSSGSRHMKKQTFVVH
AGTDTNGDIFFMEVCDDCVVLRSNIGTVYERWWYEKLINMTYCPKTKVLC
LWRRNGSETQLNKFYTKKCRELYYCVKDSMERAAARQQSIKPGPELGGEF
PVQDLKTGEGGLLQVTLEGINLKFMHNQFLKLKKW

siRNA Sequences that Target Exon 34 Region (Underlined).

An embodiment of the target region for Exon 34 is:

GGTTTTCATAGAGCTGAATCACATTAAAAAGTGCAATACAGTTCGAGGCG
TCTTTGTCCTGGAGGAATTT
5′-
GATCCCCAGAGCTGAATCACATTAAATTCAAGAGATTTAATGTGATTCAG
CTCTTTTTTA-3′
5′-
AGCTTAAAAAAGAGCTGAATCACATTAAATCTCTTGAATTTAATGTGATT
CAGCTCTGGG-3′
Oligo 4642 oligo #64/65
5′-
GATCCCCCAGTTCGAGGCGTCTTTGTTTCAAGAGAACAAAGACGCCTCGA
ACTGTTTTTA-3′
5′-
AGCTTAAAAACAGTTCGAGGCGTCTTTGTTCTCTTGAAACAAAGACGCCT
CGAACTGGGG-3′
Oligo 4649 OLIGO#62/63
5′-
GATCCCCAGGCGTCTTTGTCCTGGAGTTCAAGAGACTCCAGGACAAAGAC
GCCTTTTTTA-3′
5′-AGCTTAAAAA
AGGCGTCTTTGTCCTGGAGTCTCTTGAACTCCAGGACAAAGACGCCTGG
G-3′

Target Sequences (Regions) for Exons 21, and 26 (of IG20) that Correspond to KIAA0358

An embodiment of the target region for Exon 21 is:

AATTGTGGAACAAGCACCAGGAAGTGAAAAAGCAAAAAGCTTTGGAAAAA
CAGA

An embodiment of the target region for Exon 26 is:

AAGGGACAAAGGATCCATGTGGGACCAGTTAGAGGATGCAGCTATGGAGA
CCTTTTCTATAAG

TABLE 2
Exon 21 target regions and siRNA sequences

Position: 3527,
Binding Site: AAUUGUGGAACAAGCACCA,
Guide RNA: UGGUGCUUGUUCCACAAUU
Position: 3528,
Binding Site: AUUGUGGAACAAGCACCAG,
Guide RNA: CUGGUGCUUGUUCCACAAU
Position: 3529,
Binding Site: UUGUGGAACAAGCACCAGG,
Guide RNA: CCUGGUGCUUGUUCCACAA
Position: 3530,
Binding Site: UGUGGAACAAGCACCAGGA,
Guide RNA: UCCUGGUGCUUGUUCCACA
Position: 3531,
Binding Site: GUGGAACAAGCACCAGGAA,
Guide RNA: UUCCUGGUGCUUGUUCCAC
Position: 3532,
Binding Site: UGGAACAAGCACCAGGAAG,
Guide RNA: CUUCCUGGUGCUUGUUCCA
Position: 3533,
Binding Site: GGAACAAGCACCAGGAAGU,
Guide RNA: ACUUCCUGGUGCUUGUUCC
Position: 3534,
Binding Site: GAACAAGCACCAGGAAGUG,
Guide RNA: CACUUCCUGGUGCUUGUUC
Position: 3535,
Binding Site: AACAAGCACCAGGAAGUGA,
Guide RNA: UCACUUCCUGGUGCUUGUU
Position: 3536,
Binding Site: ACAAGCACCAGGAAGUGAA,
Guide RNA: UUCACUUCCUGGUGCUUGU
Position: 3537,
Binding Site: CAAGCACCAGGAAGUGAAA,
Guide RNA: UUUCACUUCCUGGUGCUUG
Position: 3574,
Binding Site: AAACAGAGGCCUGAAGUAA,
Guide RNA: UUACUUCAGGCCUCUGUUU
Position: 3575,
Binding Site: AACAGAGGCCUGAAGUAAU,
Guide RNA: AUUACUUCAGGCCUCUGUU
Position: 3576,
Binding Site: ACAGAGGCCUGAAGUAAUC,
Guide RNA: GAUUACUUCAGGCCUCUGU
Position: 3577,
Binding Site: CAGAGGCCUGAAGUAAUCA,
Guide RNA: UGAUUACUUCAGGCCUCUG
Position: 3578,
Binding Site: AGAGGCCUGAAGUAAUCAA,
Guide RNA: UUGAUUACUUCAGGCCUCU
Position: 3579,
Binding Site: GAGGCCUGAAGUAAUCAAA,
Guide RNA: UUUGAUUACUUCAGGCCUC

TABLE 3
Exon 26 target regions and siRNA sequences

Position: 4034,
Binding Site: GAAGGGACAAAGGAUCCAU,
Guide RNA: AUGGAUCCUUUGUCCCUUC
Position: 4035,
Binding Site: AAGGGACAAAGGAUCCAUG,
Guide RNA: CAUGGAUCCUUUGUCCCUU
Position: 4036,
Binding Site: AGGGACAAAGGAUCCAUGU,
Guide RNA: ACAUGGAUCCUUUGUCCCU
Position: 4037,
Binding Site: GGGACAAAGGAUCCAUGUG,
Guide RNA: CACAUGGAUCCUUUGUCCC
Position: 4038,
Binding Site: GGACAAAGGAUCCAUGUGG,
Guide RNA: CCACAUGGAUCCUUUGUCC
Position: 4039,
Binding Site: GACAAAGGAUCCAUGUGGG,
Guide RNA: CCCACAUGGAUCCUUUGUC
Position: 4040,
Binding Site: ACAAAGGAUCCAUGUGGGA,
Guide RNA: UCCCACAUGGAUCCUUUGU
Position: 4041,
Binding Site: CAAAGGAUCCAUGUGGGAC,
Guide RNA: GUCCCACAUGGAUCCUUUG
Position: 4042,
Binding Site: AAAGGAUCCAUGUGGGACC,
Guide RNA: GGUCCCACAUGGAUCCUUU
Position: 4043,
Binding Site: AAGGAUCCAUGUGGGACCA,
Guide RNA: UGGUCCCACAUGGAUCCUU
Position: 4044,
Binding Site: AGGAUCCAUGUGGGACCAG,
Guide RNA: CUGGUCCCACAUGGAUCCU
Position: 4045,
Binding Site: GGAUCCAUGUGGGACCAGU,
Guide RNA: ACUGGUCCCACAUGGAUCC
Position: 4046,
Binding Site: GAUCCAUGUGGGACCAGUU,
Guide RNA: AACUGGUCCCACAUGGAUC
Position: 4047,
Binding Site: AUCCAUGUGGGACCAGUUA,
Guide RNA: UAACUGGUCCCACAUGGAU
Position: 4048,
Binding Site: UCCAUGUGGGACCAGUUAG,
Guide RNA: CUAACUGGUCCCACAUGGA
Position: 4049,
Binding Site: CCAUGUGGGACCAGUUAGA,
Guide RNA: UCUAACUGGUCCCACAUGG
Position: 4050,
Binding Site: CAUGUGGGACCAGUUAGAG,
Guide RNA: CUCUAACUGGUCCCACAUG
Position: 4051,
Binding Site: AUGUGGGACCAGUUAGAGG,
Guide RNA: CCUCUAACUGGUCCCACAU
Position: 4052,
Binding Site: UGUGGGACCAGUUAGAGGA,
Guide RNA: UCCUCUAACUGGUCCCACA
Position: 4053,
Binding Site: GUGGGACCAGUUAGAGGAU,
Guide RNA: AUCCUCUAACUGGUCCCAC
Position: 4054,
Binding Site: UGGGACCAGUUAGAGGAUG,
Guide RNA: CAUCCUCUAACUGGUCCCA
Position: 4055,
Binding Site: GGGACCAGUUAGAGGAUGC,
Guide RNA: GCAUCCUCUAACUGGUCCC
Position: 4056,
Binding Site: GGACCAGUUAGAGGAUGCA,
Guide RNA: UGCAUCCUCUAACUGGUCC
Position: 4057,
Binding Site: GACCAGUUAGAGGAUGCAG,
Guide RNA: CUGCAUCCUCUAACUGGUC
Position: 4058,
Binding Site: ACCAGUUAGAGGAUGCAGC,
Guide RNA: GCUGCAUCCUCUAACUGGU
Position: 4059,
Binding Site: CCAGUUAGAGGAUGCAGCU,
Guide RNA: AGCUGCAUCCUCUAACUGG
Position: 4060,
Binding Site: CAGUUAGAGGAUGCAGCUA,
Guide RNA: UAGCUGCAUCCUCUAACUG
Position: 4061,
Binding Site: AGUUAGAGGAUGCAGCUAU,
Guide RNA: AUAGCUGCAUCCUCUAACU
Position: 4062,
Binding Site: GUUAGAGGAUGCAGCUAUG,
Guide RNA: CAUAGCUGCAUCCUCUAAC
Position: 4063,
Binding Site: UUAGAGGAUGCAGCUAUGG,
Guide RNA: CCAUAGCUGCAUCCUCUAA
Position: 4064,
Binding Site: UAGAGGAUGCAGCUAUGGA,
Guide RNA: UCCAUAGCUGCAUCCUCUA
Position: 4065,
Binding Site: AGAGGAUGCAGCUAUGGAG,
Guide RNA: CUCCAUAGCUGCAUCCUCU
Position: 4066,
Binding Site: GAGGAUGCAGCUAUGGAGA,
Guide RNA: UCUCCAUAGCUGCAUCCUC
Position: 4067,
Binding Site: AGGAUGCAGCUAUGGAGAC,
Guide RNA: GUCUCCAUAGCUGCAUCCU
Position: 4068,
Binding Site: GGAUGCAGCUAUGGAGACC,
Guide RNA: GGUCUCCAUAGCUGCAUCC
Position: 4069,
Binding Site: GAUGCAGCUAUGGAGACCU,
Guide RNA: AGGUCUCCAUAGCUGCAUC
Position: 4070,
Binding Site: AUGCAGCUAUGGAGACCUU,
Guide RNA: AAGGUCUCCAUAGCUGCAU
Position: 4071,
Binding Site: UGCAGCUAUGGAGACCUUU,
Guide RNA: AAAGGUCUCCAUAGCUGCA
Position: 4088,
Binding Site: UUUCUAUAAGCAAAGAGCG,
Guide RNA: CGCUCUUUGCUUAUAGAAA
Position: 4089,
Binding Site: UUCUAUAAGCAAAGAGCGU,
Guide RNA: ACGCUCUUUGCUUAUAGAA
Position: 4090,
Binding Site: UCUAUAAGCAAAGAGCGUU,
Guide RNA: AACGCUCUUUGCUUAUAGA
Position: 4091,
Binding Site: CUAUAAGCAAAGAGCGUUC,
Guide RNA: GAACGCUCUUUGCUUAUAG
Position: 4092,
Binding Site: UAUAAGCAAAGAGCGUUCU,
Guide RNA: AGAACGCUCUUUGCUUAUA
Position: 4093,
Binding Site: AUAAGCAAAGAGCGUUCUA,
Guide RNA: UAGAACGCUCUUUGCUUAU
Position: 4094,
Binding Site: UAAGCAAAGAGCGUUCUAC,
Guide RNA: GUAGAACGCUCUUUGCUUA
Position: 4095,
Binding Site: AAGCAAAGAGCGUUCUACU,
Guide RNA: AGUAGAACGCUCUUUGCUU
Position: 4096,
Binding Site: AGCAAAGAGCGUUCUACUU,
Guide RNA: AAGUAGAACGCUCUUUGCU

TABLE 4
Exon 34 target regions and siRNA sequences

Position: 4914,
Binding Site: AAAGUGCAAUACAGUUCGA,
Guide RNA: UCGAACUGUAUUGCACUUU
Position: 4915,
Binding Site: AAGUGCAAUACAGUUCGAG,
Guide RNA: CUCGAACUGUAUUGCACUU
Position: 4916,
Binding Site: AGUGCAAUACAGUUCGAGG,
Guide RNA: CCUCGAACUGUAUUGCACU
Position: 4917,
Binding Site: GUGCAAUACAGUUCGAGGC,
Guide RNA: GCCUCGAACUGUAUUGCAC
Position: 4918,
Binding Site: UGCAAUACAGUUCGAGGCG,
Guide RNA: CGCCUCGAACUGUAUUGCA
Position: 4919,
Binding Site: GCAAUACAGUUCGAGGCGU,
Guide RNA: ACGCCUCGAACUGUAUUGC
Position: 4920,
Binding Site: CAAUACAGUUCGAGGCGUC,
Guide RNA: GACGCCUCGAACUGUAUUG
Position: 4921,
Binding Site: AAUACAGUUCGAGGCGUCU,
Guide RNA: AGACGCCUCGAACUGUAUU
Position: 4922,
Binding Site: AUACAGUUCGAGGCGUCUU,
Guide RNA: AAGACGCCUCGAACUGUAU
Position: 4923,
Binding Site: UACAGUUCGAGGCGUCUUU,
Guide RNA: AAAGACGCCUCGAACUGUA
Position: 4924,
Binding Site: ACAGUUCGAGGCGUCUUUG,
Guide RNA: CAAAGACGCCUCGAACUGU
Position: 4925,
Binding Site: CAGUUCGAGGCGUCUUUGU,
Guide RNA: ACAAAGACGCCUCGAACUG
Position: 4926,
Binding Site: AGUUCGAGGCGUCUUUGUC,
Guide RNA: GACAAAGACGCCUCGAACU
Position: 4927,
Binding Site: GUUCGAGGCGUCUUUGUCC,
Guide RNA: GGACAAAGACGCCUCGAAC
Position: 4928,
Binding Site: UUCGAGGCGUCUUUGUCCU,
Guide RNA: AGGACAAAGACGCCUCGAA
Position: 4929,
Binding Site: UCGAGGCGUCUUUGUCCUG,
Guide RNA: CAGGACAAAGACGCCUCGA
Position: 4930,
Binding Site: CGAGGCGUCUUUGUCCUGG,
Guide RNA: CCAGGACAAAGACGCCUCG
Position: 4931,
Binding Site: GAGGCGUCUUUGUCCUGGA,
Guide RNA: UCCAGGACAAAGACGCCUC
Position: 4932,
Binding Site: AGGCGUCUUUGUCCUGGAG,
Guide RNA: CUCCAGGACAAAGACGCCU
Position: 4933,
Binding Site: GGCGUCUUUGUCCUGGAGG,
Guide RNA: CCUCCAGGACAAAGACGCC
Position: 4934,
Binding Site: GCGUCUUUGUCCUGGAGGA,
Guide RNA: UCCUCCAGGACAAAGACGC
Position: 4935,
Binding Site: CGUCUUUGUCCUGGAGGAA,
Guide RNA: UUCCUCCAGGACAAAGACG
Position: 4936,
Binding Site: GUCUUUGUCCUGGAGGAAU,
Guide RNA: AUUCCUCCAGGACAAAGAC
Position: 4937,
Binding Site: UCUUUGUCCUGGAGGAAUU,
Guide RNA: AAUUCCUCCAGGACAAAGA
Position: 4938,
Binding Site: CUUUGUCCUGGAGGAAUUU,
Guide RNA: AAAUUCCUCCAGGACAAAG
Position: 4939,
Binding Site: UUUGUCCUGGAGGAAUUUG,
Guide RNA: CAAAUUCCUCCAGGACAAA
Position: 4940,
Binding Site: UUGUCCUGGAGGAAUUUGU,
Guide RNA: ACAAAUUCCUCCAGGACAA
Position: 4941,
Binding Site: UGUCCUGGAGGAAUUUGUU,
Guide RNA: AACAAAUUCCUCCAGGACA
Position: 4942,
Binding Site: GUCCUGGAGGAAUUUGUUC,
Guide RNA: GAACAAAUUCCUCCAGGAC
Position: 4943,
Binding Site: UCCUGGAGGAAUUUGUUCC,
Guide RNA: GGAACAAAUUCCUCCAGGA
Position: 4944,
Binding Site: CCUGGAGGAAUUUGUUCCU,
Guide RNA: AGGAACAAAUUCCUCCAGG
Position: 4945,
Binding Site: CUGGAGGAAUUUGUUCCUG,
Guide RNA: CAGGAACAAAUUCCUCCAG
Position: 4946,
Binding Site: UGGAGGAAUUUGUUCCUGA,
Guide RNA: UCAGGAACAAAUUCCUCCA
Position: 4947,
Binding Site: GGAGGAAUUUGUUCCUGAA,
Guide RNA: UUCAGGAACAAAUUCCUCC

The binding site sequences and guide RNA sequences are exemplary for Exons 21, 26, and 34. Similarly, corresponding shRNA vectors that have complementary or reverse complementary DNA sequences to express shRNA and siRNA can be readily designed based on the binding sites and guide RNA sequences provided herein.

1. A composition comprising a short-interfering RNA (siRNA) that specifically down regulates the expression of an IG20 splice variant KIAA0358 in a neuroblastoma cell.

2. The composition of claim 1, wherein the siRNA targets Exon 21 or Exon 26 of the IG20 gene.

3. The composition of claim 1, wherein the siRNA comprises a nucleic acid sequence selected from Table 2 that targets Exon 21 or a nucleic acid sequence selected from Table 3 that targets Exon 26.

4. The composition of claim 2, wherein the siRNA targets Exon 21 of the IG20 gene in a region comprising a nucleotide sequence AATTGTGGAACAAGCACCAGGAAGTGAAAAAGCAAAAAGCTTTGGAAAAACAGA (SEQ ID NO: 1) or targets Exon 26 of the IG20 gene in a region comprising a nucleotide sequence AAGGGACAAAGGATCCATGTGGGACCAGTTAGAGGATGCAGCTATGGAGACCTTTT CTATAAG (SEQ ID NO: 2).

5. A composition comprising a short-interfering RNA (siRNA) that specifically down regulates the expression of splice variants of IG20, the variants comprising IG20pa, MADD, IG20-SV2, DENN-SV, KIAA0358 except IG20-SV4 in a neuroblastoma cell.

6. The composition of claim 5, wherein the siRNA targets exon 13L and 34 of the IG20 gene.

7. The composition of claim 6 wherein the siRNA targets Exon 13L of the IG20 gene in a region comprising a nucleotide sequence CGGCGAATCTATGACAATC (SEQ ID NO: 3) and targets Exon 34 of the IG20 gene in a region comprising a nucleotide sequence GGTTTTCATAGAGCTGAATCACATTAAAAAGTGCAATACAGTTCGAGGCGTCTTTGT CCTGGAGGAATTT (SEQ ID NO: 4).

8. A purified or isolated short-interfering RNA (siRNA) molecule that specifically down regulates the expression of an IG20 splice variant KIAA0358 in a neuroblastoma cell.

9. A purified or isolated short-interfering RNA (siRNA) that specifically down regulates the expression of splice variants of IG20 comprising IG20pa, MADD, IG20-SV2, DENN-SV, KIAA0358 except IG20-SV4 in a neuroblastoma cell.

10. A purified or isolated vector expressing the siRNA of claim 8, wherein the siRNA comprises a nucleic acid sequence selected from Table 2 that targets Exon 21 or a nucleic acid sequence selected from Table 3 that targets Exon 26.

11. A purified or isolated vector expressing the siRNA of claim 9, wherein the siRNA comprises a nucleic acid sequence 5′-AGAGCTGAATCACATTAAA-3′ (SEQ ID NO: 5) that targets Exon 13L and comprises a nucleic acid sequence 5′-AGAGCTGAATCACATTAAA-3′ (SEQ ID NO: 5) that targets Exon 34 of the IG20 gene.

12. (canceled)

13. The composition of claim 1 comprising a short-interfering RNA (siRNA) to specifically down regulate an IG20 splice variant KIAA0358 by enhancing apoptosis in a neuroblastoma cell.

14. (canceled)

15. The method of claim 24 wherein the siRNA targets exon 21 or exon 26 of the IG20 gene to down regulate expression of KIAA0358.

16. The method of claim 15 wherein the siRNA targets Exon 21 of the IG20 gene in a region comprising a nucleotide sequence AATTGTGGAACAAGCACCAGGAAGTGAAAAAGCAAAAAGCTTTGGAAAAACAGA (SEQ ID NO: 1) or targets Exon 26 of the IG20 gene in a region comprising a nucleotide sequence AAGGGACAAAGGATCCATGTGGGACCAGTTAGAGGATGCAGCTATGGAGACCTTTT CTATAAG (SEQ ID NO: 2).

17. The method of claim 15, wherein the siRNA comprises a nucleic acid sequence selected from Table 2 that targets Exon 21 or a nucleic acid sequence selected from Table 3 that targets Exon 26.

18. (canceled)

19. The composition of claim 5 comprising a short-interfering RNA (siRNA) to specifically down regulate the expression of splice variants of IG20 comprising IG20pa, MADD, IG20-SV2, DENN-SV, KIAA0358 except IG20-SV4 for use to enhance apoptosis in a neuroblastoma cell.

20. (canceled)

21. The method of claim 24, wherein the siRNA targets Exon 13L and Exon 34 of the IG20 gene to down regulate expression of IG20pa, MADD, IG20-SV2, DENN-SV, KIAA03858 except IG20-SV4.

22. (canceled)

23. The method of claim 21, wherein the siRNA targets Exon 13L of the IG20 gene in a region comprising a nucleotide sequence CGGCGAATCTATGACAATC (SEQ ID NO: 3) and targets Exon 34 of the IG20 gene in a region comprising a nucleotide sequence GGTTTTCATAGAGCTGAATCACATTAAAAAGTGCAATACAGTTCGAGGCGTCTTTGT CCTGGAGGAATTT (SEQ ID NO: 4).

24. A method to enhance apoptosis in neuroblastoma cells, the method comprising:
(a) specifically down regulating the expression of an IG20 splice variant KIAA0358; or
(b) specifically down regulating the expression of splice variants of IG20 comprising IG20pa, MADD, IG20-S V2, DENN-SV, KIAA0358 except 1620-SV4; or
(c) providing a composition comprising a cDNA sequence for expressing an IG20 splice variant IG20-SV4 or a domain thereof in a neuroblastoma cell.

25. The method of claim 24, wherein the neuroblastoma cells are further exposed to TNFα or interferon-γ treatment.

26. The method of claim 24, further comprising providing a cytotoxic agent.

27. A method to ameliorate one or more conditions associated with a neurodegenerative disorder by expressing a nucleotide sequence or a coding for KIAA0358 or a coding fragment thereof.

28. The method of claim 27, wherein the expression of the nucleotide sequence of KIAA0358 or the coding fragment thereof reduces cell death.

29. The method of claim 27, wherein the neurodegenerative disorder is selected from the group consisting of multiple sclerosis, Parkinson's disease, and Alzheimer's disease.

30. An engineered mammalian virus comprising the vector of claim 10.

31. The virus of claim 30 is selected from the group consisting of adenovirus, adeno-associated virus, herpes virus, and lentivirus.

32. A neural cell transfected with the virus of claim 31.

33. (canceled)

34. An engineered mammalian virus comprising the vector of claim 11.
